(19) United States
(12) Patent Application Publication
(10) Pub. No.: US 2019/0175571 A1
(43) Pub. Date: Jun. 13, 2019

(54) IMIDAZOLE DERIVATIVES AND THEIR USE IN THE TREATMENT OF AUTOIMMUNE OR INFLAMMATORY DISEASES OR CANCERS

(71) Applicant: GLAXOSMITHKLINE INTELLECTUAL PROPERTY (NO.2) LIMITED, Brentford, Middlesex (GB)

(72) Inventors: Andrew BAXTER, Stevenage, Hertfordshire (GB); John Alexander BROWN, Cambridge, MA (US); David HIRST, Stevenage, Hertfordshire (GB); Philip HUMPHREYS, Stevenage, Hertfordshire (GB); Katherine Louise JONES, Stevenage, Hertfordshire (GB); Vipulkumar Kantibhai PATEL, Stevenage, Hertfordshire (GB)

(21) Appl. No.: 16/326,991

(22) PCT Filed: Aug. 31, 2017
(86) PCT No.: PCT/EP2017/071868

§ 371 Date: Feb. 21, 2019

(30) Foreign Application Priority Data
Sep. 2, 2016 (GB) 1614934.6

(51) Int. Cl. A61K 31/4439; A61P 29/00; A61P 35/00; A61K 31/443; A61K 31/4433

(52) U.S. Cl. CPC A61K 31/4439; A61P 29/00; A61K 31/4433; A61K 31/443; A61P 35/00

(57) ABSTRACT

Compounds of formula (I) and salts thereof:

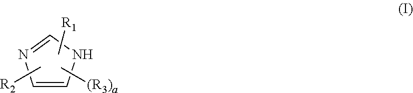

wherein R₁, R₂, R₃ and a are as defined herein. Compounds of formula (I) and salts thereof have been found to inhibit the binding of the BET family of bromodomain containing proteins to, for example, acetylated lysine residues and thus may have use in therapy, for example in the treatment of autoimmune and inflammatory diseases, such as rheumatoid arthritis; and cancers.

### FIELD OF THE INVENTION

The present invention relates to compounds, compositions containing them, and to their use in the treatment of various disorders in particular inflammatory and autoimmune diseases, such as rheumatoid arthritis; and cancers.

### BACKGROUND TO THE INVENTION

The genomes of eukaryotic organisms are highly organised within the nucleus of the cell. The long strands of duplex DNA are wrapped around an octomer of histone proteins (most usually comprising two copies of histones H2A, H2B, H3 and H4) to form a nucleosome. This basic unit is then further compressed by the aggregation and folding of nucleosomes to form a highly condensed chromatin structure. A range of different states of condensation are possible, and the tightness of this structure varies during the cell cycle, being most compact during the process of cell division. Chromatin structure plays a critical role in regulating gene transcription, which cannot occur efficiently from highly condensed chromatin. The chromatin structure is controlled by a series of post translational modifications to histone proteins, notably histones H3 and H4, and most commonly within the histone tails which extend beyond the core nucleosome structure. These modifications include acetylation, methylation, phosphorylation, ubiquitinylation, and SUMOylation. These epigenetic marks are written and erased by specific enzymes, which place tags on specific residues within the histone tail, thereby forming an epigenetic code, which is then interpreted by the cell to allow regulation of gene expression.

Histone acetylation is most usually associated with the activation of gene transcription, as the modification relaxes the interaction of the DNA and the histone octomer by changing the electrostatics. In addition to this physical change, specific proteins recognise and bind to acetylated lysine residues within histones to read the epigenetic code. Bromodomains are small (˜110 amino acid) distinct domains within proteins that bind to acetylated lysine resides commonly but not exclusively in the context of histones. There is a family of around 50 proteins known to contain bromodomains, and they have a range of functions within the cell.

The BET family of bromodomain containing proteins comprises 4 proteins (BRD2, BRD3, BRD4 and BRDT) which contain tandem bromodomains capable of binding to two acetylated lysine residues in close proximity, increasing the specificity of the interaction. Numbering from the N-terminal end of each BET protein the tandem bromodomains are typically labelled Binding Domain 1 (BD1) and Binding Domain 2 (BD2) (Chung et al, J Med. Chem., 2011, 54, 3827-3838).

Inhibiting the binding of a BET protein to acetylated lysine residues has the potential to ameliorate progression of several diseases, including but not limited to, cancer (Dawson M. A. et al, Nature, 2011: 478(7370):529-33; Wyce, A. et al, Oncotarget. 2013: 4(12):2419-29), sepsis (Nicodeme E. et al, Nature, 2010: 468(7327):1119-23), autoimmune and inflammatory diseases such as rheumatoid arthritis and multiple sclerosis (Mele D. A. et al, Journal of Experimental Medicine, 2013: 210(11):2181-90), heart failure (Anand P. et al, Cell, 2013: 154(3):569-82), and lung fibrosis (Tang X. et al, Molecular Pharmacology, 2013: 83(1): 283-293).

There exists a need for chemical compounds which inhibit the activity of bromodomains, in particular compounds that inhibit the binding of BET proteins to acetylated lysine residues and hence have utility in the treatment of, for example, autoimmune and inflammatory diseases, and cancers.

### SUMMARY OF THE INVENTION

In a first aspect, the present invention provides a compound of formula (I), or a salt thereof:

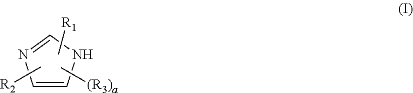

wherein
R₁ represents

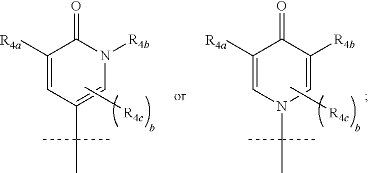

R₂ is hydrogen, C₁₋₆alkyl, C₁₋₆alkoxy, C₃₋₇cycloalkyl, heterocycloalkyl or —CHR₅(CH₂)_{c}R₆;
each R₃ is independently selected from the group consisting of halogen, —CN, C₁₋₃alkyl, C₁₋₃alkoxy, —NO₂, —CONR₇R₈, —NR₇COR₈, —OCOR₈, —CO₂R₈, —SO₂NR₇R₈, —NR₇SO₂R₈, —SO₂R₈, —R₈, —NR₇R₈, and —OR₈, with the proviso that when a is 2, one R₃ is selected from the group consisting of halogen, —CN, C₁₋₃alkyl and C₁₋₃alkoxy;
R₄ₐ is hydrogen, C₁₋₃alkyl, C₁₋₃alkoxy, halogen, —CN, —OH, or —NR₉R₁₀;
R_{4b} is hydrogen or C₁₋₃alkyl;
each R_{4c} is independently selected from the group consisting of C₁₋₃alkyl, C₁₋₃alkoxy, halogen, —CN, —OH, and —NR₉R₁₀;
R₅ is hydrogen, C₁₋₃alkyl, or —(CH₂)_{d}OR₁₁;
R₆ is hydrogen, C₁₋₃alkyl, —(CH₂)_{d}OR₁₁, C₃₋₇cycloalkyl, or heterocycloalkyl, wherein the C₁₋₃alkyl, —(CH₂)_{d}OR₁₁, C₃₋₇cycloalkyl, heterocycloalkyl groups can be optionally substituted with one or two substituents independently selected from the group consisting of C₁₋₃alkyl, C₁₋₃alkoxy, halogen, —CH₂OH, —COOH, and —COCH₃;
R₇ is hydrogen or C₁₋₃alkyl and R₈ is —Y—Z, or when R₃ is —CONR₇R₈, R₇ and R₈ together with the nitrogen to which they are attached may form a heterocycloalkyl, wherein the heterocycloalkyl group can be optionally substituted with one or two groups independently selected from C₁₋₃alkyl, halogen, —NH₂, —CH₂NH₂, —CO₂H, —OH, —CN, and —CH₂OH;
Y is a bond or C₁₋₃alkylene, wherein the C₁₋₃alkylene group can be optionally substituted with one or two groups independently selected from C₁₋₃alkyl;
Z is hydrogen, C₁₋₃alkyl, C₃₋₇cycloalkyl, heterocycloalkyl, aryl, heteroaryl, —SO₂NR₁₂R₁₃, —NR₁₂SO₂R₁₃, —SO₂R₁₂, or —NR₁₂R₁₃, wherein C₁₋₃alkyl, C₃₋₇cycloalkyl, heterocycloalkyl, aryl or heteroaryl can be optionally substituted with one or two groups independently selected from C₁₋₃alkyl, C₁₋₃alkoxy, halogen, —NH₂, —CH₂NH₂, —CO₂H, —OH, —CN, and —CH₂OH;
each R₉ is independently selected from hydrogen or CH₃;
each R₁₀ is independently selected from hydrogen or C₁₋₃alkyl;
R₁₁ is hydrogen or C₁₋₃alkyl;
R₁₂ is hydrogen or C₁₋₃alkyl;
R₁₃ is hydrogen or C₁₋₃alkyl;
a represents 0, 1 or 2;
b represents 0, 1 or 2;
each c and d independently represent 0 or 1.

Compounds of the invention have been found to inhibit the binding of bromodomain containing proteins; in particular, the binding of the BET family of bromodomain containing proteins to, for example, acetylated lysine residues. Compounds of formula (I), or pharmaceutically acceptable salts thereof, may thus have use in therapy, for example in the treatment of autoimmune and inflammatory diseases, such as rheumatoid arthritis; and cancers.

The present invention is further directed to methods of treatment of autoimmune and inflammatory diseases and cancers through inhibition of the function of bromodomain containing proteins, for example members of the BET family of bromodomain containing proteins, which comprises administering to a subject in need thereof, a therapeutically effective amount of a compound of formula (I), or a pharmaceutically acceptable salt thereof.

In a further aspect, the present invention is directed to pharmaceutical compositions comprising a compound of formula (I), or a pharmaceutically acceptable salt thereof, and one or more pharmaceutically acceptable excipients.

### BRIEF DESCRIPTION OF THE DRAWINGS

FIG. 1 shows an X-ray powder diffraction pattern of Example 30a.

FIG. 2 shows a Raman spectrum of Example 30a.

FIG. 3 shows a thermogravimetric analysis thermogram (TGA) of Example 30a.

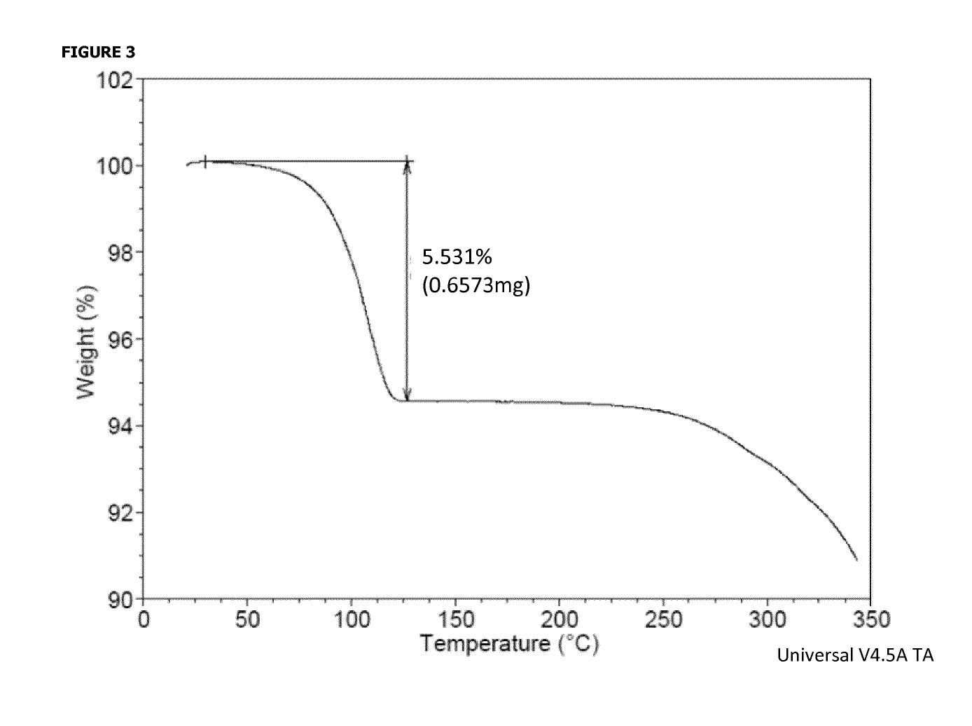

### DETAILED DESCRIPTION OF THE INVENTION

### Definitions

As used herein, the term “bromodomain” refers to evolutionary and structurally conserved modules (approximately 110 amino acids in length) that bind acetylated lysine residues, such as those on the N-terminal tails of histones. They are protein domains that are found as part of much larger bromodomain containing proteins (BCPs), many of which have roles in regulating gene transcription and/or chromatin remodelling. The human genome encodes for at least 57 bromodomains.

As used herein, the term “BET” refers to the bromodomain and extraterminal domain family of bromodomain containing proteins which include BRD2, BRD3, BRD4 and BRDT.

As used herein, the term “BET inhibitor” refers to a compound that is capable of inhibiting the binding of one or more BET family bromodomain containing proteins (e.g. BRD2, BRD3, BRD4 or BRDT) to, for example, acetylated lysine residues.

As used herein, the term “alkyl” refers to a saturated hydrocarbon chain, straight or branched, having the specified number of carbon atoms. For example, C₁₋₆ alkyl refers to an alkyl group having from 1 to 6 carbon atoms. Unless otherwise stated, alkyl groups are unsubstituted. The term “alkyl” includes, but is not limited to, methyl, ethyl, propyl (n-propyl and isopropyl), butyl (n-butyl, sec-butyl, isobutyl and tert-butyl), pentyl, and hexyl.

As used herein, the term “alkylene” refers to a divalent radical derived from a straight or branched, saturated hydrocarbon chain of, for example, 1 to 3 carbon atoms (C₁₋₃alkylene). Examples of alkylene include —CH₂—, —CH₂CH₂—, and —CH₂CH₂CH₂—.

As used herein, the term “alkoxy” refers to an —O-alkyl group wherein “alkyl” is defined above.

As used herein, the term “C₃₋₇cycloalkyl” refers to a saturated, monocyclic, hydrocarbon ring having 3 (cyclopropyl), 4 (cyclobutyl), 5 (cyclopentyl), 6 (cyclohexyl) or 7 (cycloheptyl) carbon atoms.

As used herein, the term “halogen” refers to fluoro, chloro, bromo and iodo.

As used herein, the term “heterocycloalkyl” refers to a saturated or unsaturated 3 to 7 membered monocyclic or bicyclic ring, which must contain 1 or 2 non-carbon atoms, which are selected from nitrogen, oxygen, and sulfur. Heterocycloalkyl groups may contain one or more C(O), S(O) or SO₂ groups. However, heterocycloalkyl groups are not aromatic. Heterocycloalkyl groups containing more than one heteroatom may contain different heteroatoms. “5 or 6 membered heterocycloalkyl” refers to a saturated or unsaturated 5 or 6 membered monocyclic ring, which must contain 1 or 2 non-carbon atoms, which are selected from nitrogen, oxygen, and sulfur.

Heterocycloalkyl includes, but is not limited to, pyrrolidine, piperidine, piperazine, oxetane, tetrahydrofuran, tetrahydro-2H-pyran, morpholine, morpholine-3-one, piperidin-2-one, pyrimidine-2,4(1H,3H)-dione, thiomorpholine, and thiomorpholine 1,1-dioxide.

As used herein, the term “aryl” refers to a monocyclic or bicyclic, hydrocarbon, aromatic radical. Aryl includes, for example, phenyl and naphthyl.

As used herein, the term “heteroaryl” refers to a monocyclic or bicyclic, aromatic radical containing one or more heteroatoms selected from S, N and O. Illustrative examples of heteroaryl useful in the present invention include, but are not limited to, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, triazinyl, benzofuranyl, isobenzofuryl, 2,3-dihydrobenzofuryl, 1,3-benzodioxolyl, dihydrobenzodioxinyl, benzothienyl, indolizinyl, indolyl, isoindolyl, dihydroindolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, imidazopyridinyl, pyrazolopyridinyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

As used herein, the phrase “optionally substituted” indicates that a group may be unsubstituted or substituted with one or more substituents as defined herein. “Substituted” in reference to a group indicates that a hydrogen atom attached to a member atom within a group is replaced by one of the defined substituents.

As used herein, the term “pharmaceutically acceptable salts” refers to salts that retain the desired biological activity of the subject compound and exhibit minimal undesired toxicological effects.

These pharmaceutically-acceptable salts may be prepared in situ during the final isolation and purification of the compound, or by separately reacting the purified compound in its free acid or free base form with a suitable base or acid, respectively. Furthermore, pharmaceutically-acceptable salts of the compound of formula (I) may be prepared during further processing of the free acid or base form, for example in situ during manufacture into a pharmaceutical formulation.

As used herein, the term “treatment” refers to prophylaxis of the condition, ameliorating or stabilising the specified condition, reducing or eliminating the symptoms of the condition, slowing or eliminating the progression of the condition, and preventing or delaying reoccurrence of the condition in a previously afflicted patient or subject. In one embodiment, treatment refers to ameliorating or stabilising a specified condition, reducing or eliminating the symptoms of the condition, or slowing or eliminating the progression of the condition.

As used herein, the term “therapeutically effective amount” refers to the quantity of a compound of formula (I), or a pharmaceutically acceptable salt thereof, which will elicit the desired biological response in an animal or human body.

As used herein, the term “subject” refers to an animal or human body.

It is to be understood that references herein to “compound(s) of the invention” mean a compound of formula (I) as the free base, or as a salt, for example a pharmaceutically acceptable salt.

### STATEMENT OF THE INVENTION

In a first aspect, the present invention provides a compound of formula (I), or a salt thereof:

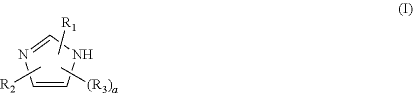

wherein
R₁ represents

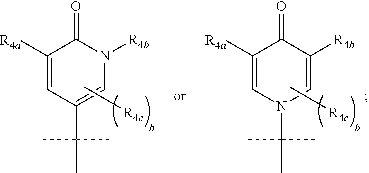

R₂ is hydrogen, C₁₋₆alkyl, C₁₋₆alkoxy, C₃₋₇cycloalkyl, heterocycloalkyl or —CHR₅(CH₂)_{c}R₆; each R₃ is independently selected from the group consisting of halogen, —CN, C₁₋₃alkyl, C₁₋₃alkoxy, —NO₂, —CONR₇R₈, —NR₇COR₈, —OCOR₈, —CO₂R₈, —SO₂NR₇R₈, —NR₇SO₂R₈, —SO₂R₈, —R₈, —NR₇R₈, and —OR₈, with the proviso that when a is 2, one R₃ is selected from the group consisting of halogen, —CN, C₁₋₃alkyl and C₁₋₃alkoxy;
R₄ₐ is hydrogen, C₁₋₃alkyl, C₁₋₃alkoxy, halogen, —CN, —OH, or —NR₉R₁₀;
R_{4b} is hydrogen or C₁₋₃alkyl;
each R_{4c} is independently selected from the group consisting of C₁₋₃alkyl, C₁₋₃alkoxy, halogen, —CN, —OH, and —NR₉R₁₀;
R₅ is hydrogen, C₁₋₃alkyl, or —(CH₂)_{d}OR₁₁;
R₆ is hydrogen, C₁₋₃alkyl, —(CH₂)_{d}OR₁₁, C₃₋₇cycloalkyl, or heterocycloalkyl, wherein the C₁₋₃alkyl, —(CH₂)_{d}OR₁₁, C₃₋₇cycloalkyl, heterocycloalkyl groups can be optionally substituted with one or two substituents independently selected from the group consisting of C₁₋₃alkyl, C₁₋₃alkoxy, halogen, —CH₂OH, —COOH, and —COCH₃;
R₇ is hydrogen or C₁₋₃alkyl and R₈ is —Y—Z, or when R₃ is —CONR₇R₈, R₇ and R₈ together with the nitrogen to which they are attached may form a heterocycloalkyl, wherein the heterocycloalkyl group can be optionally substituted with one or two groups independently selected from C₁₋₃alkyl, halogen, —NH₂, —CH₂NH₂, —CO₂H, —OH, —CN, and —CH₂OH;
Y is a bond or C₁₋₃alkylene, wherein the C₁₋₃alkylene group can be optionally substituted with one or two groups independently selected from C₁₋₃alkyl;
Z is hydrogen, C₁₋₃alkyl, C₃₋₇cycloalkyl, heterocycloalkyl, aryl, heteroaryl, —SO₂NR₁₂R₁₃, —NR₁₂SO₂R₁₃, —SO₂R₁₂, or —NR₁₂R₁₃, wherein C₁₋₃alkyl, C₃₋₇cycloalkyl, heterocycloalkyl, aryl or heteroaryl can be optionally substituted with one or two groups independently selected from C₁₋₃alkyl, C₁₋₃alkoxy, halogen, —NH₂, —CH₂NH₂, —CO₂H, —OH, —CN, and —CH₂OH;
each R₉ is independently selected from hydrogen or CH₃;
each R₁₀ is independently selected from hydrogen or C₁₋₃alkyl;
R₁₁ is hydrogen or C₁₋₃alkyl;
R₁₂ is hydrogen or C₁₋₃alkyl;
R₁₃ is hydrogen or C₁₋₃alkyl;
a represents 0, 1 or 2;
b represents 0, 1 or 2;
each c and d independently represent 0 or 1.

In one embodiment, the present invention provides a compound of formula (Ia)-(Ie), or a salt thereof:

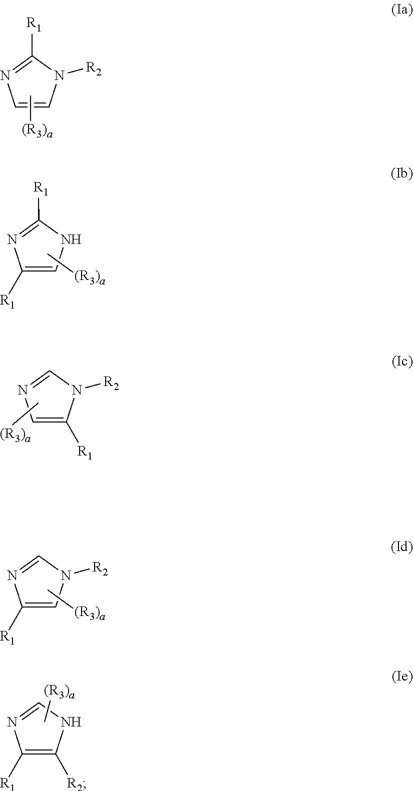

wherein R₁, R₂, R₃ and a are as defined hereinabove for a compound of formula (I).

In a further embodiment, the present invention provides a compound of formula (Ia), (Ic) or (Ie), or a salt thereof:

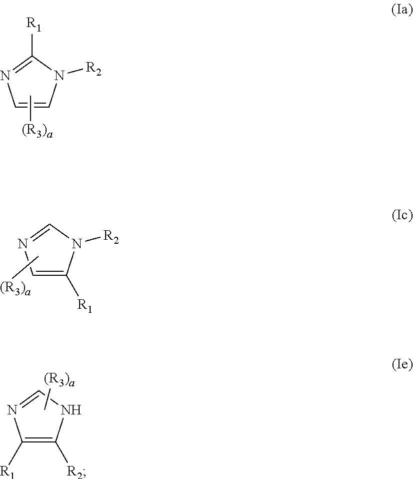

wherein R₁, R₂, R₃ and a are as defined hereinabove for a compound of formula (I).

In a further embodiment, the present invention provides a compound of formula (Ia), or a salt thereof:

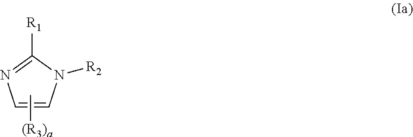

wherein R₁, R₂, R₃ and a are as defined hereinabove for a compound of formula (I).

In one embodiment, the present invention provides compounds of formula (I), or salts thereof:

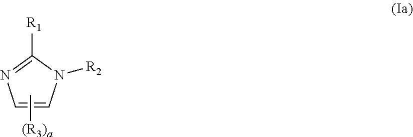

wherein
R₁ represents

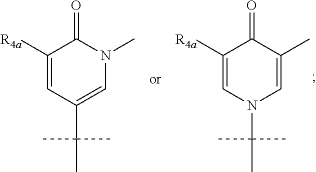

R₂ is hydrogen, C₁₋₆alkyl, C₁₋₆alkoxy, C₃₋₇cycloalkyl, heterocycloalkyl or —CHR₅(CH₂)_{c}R₆;
each R₃ is independently selected from the group consisting of halogen, —CN, C₁₋₃alkyl, C₁₋₃alkoxy, —NO₂, —CONR₇R₈, —NR₇COR₈, —OCOR₈, —CO₂R₈, —SO₂NR₇R₈, —NR₇SO₂R₈, —SO₂R₈, —R₈, —NR₇R₈, and —OR₈, with the proviso that when a is 2, one R₃ is selected from the group consisting of halogen, —CN, C₁₋₃alkyl and C₁₋₃alkoxy;
R₄ₐ is hydrogen, CH₃ or OCH₃;
R₅ is hydrogen, C₁₋₃alkyl, or —(CH₂)_{d}OR₁₁;
R₆ is hydrogen, C₁₋₃alkyl, —(CH₂)_{d}OR₁₁, C₃₋₇cycloalkyl or heterocycloalkyl, wherein the C₁₋₃alkyl, —(CH₂)_{d}OR₁₁, C₃₋₇cycloalkyl, heterocycloalkyl groups can be optionally substituted with one or two substituents independently selected from the group consisting of C₁₋₃alkyl, C₁₋₃alkoxy, halogen, —CH₂OH, —COOH, and —COCH₃;
R₇ is hydrogen or C₁₋₃alkyl and R₈ is —Y—Z, or when R₃ is —CONR₇R₈, R₇ and R₈ together with the nitrogen to which they are attached may form a heterocycloalkyl, wherein the heterocycloalkyl group can be optionally substituted with one or two groups independently selected from C₁₋₃alkyl, halogen, —NH₂, —CH₂NH₂, —CO₂H, —OH, —CN, and —CH₂OH;
Y is a bond or C₁₋₃alkylene, wherein the C₁₋₃alkylene group can be optionally substituted with one or two groups independently selected from C₁₋₃alkyl;
Z is hydrogen, C₁₋₃alkyl, C₃₋₇cycloalkyl, heterocycloalkyl, aryl, heteroaryl, —SO₂NR₁₂R₁₃, —NR₁₂SO₂R₁₃, —SO₂R₁₂, or —NR₁₂R₁₃, wherein C₁₋₃alkyl, C₃₋₇cycloalkyl, heterocycloalkyl, aryl or heteroaryl can be optionally substituted with one or two groups independently selected from C₁₋₃alkyl, C₁₋₃alkoxy, halogen, —NH₂, —CH₂NH₂, —CO₂H, —OH, —CN, and —CH₂OH;
R₁₁ is hydrogen or C₁₋₃alkyl;
R₁₂ is hydrogen or C₁₋₃alkyl;
R₁₃ is hydrogen or C₁₋₃alkyl;
a represents 0, 1 or 2;
each c and d independently represent 0 or 1.

In one embodiment, R₁ represents

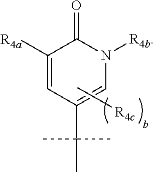

In one embodiment, R₁ represents

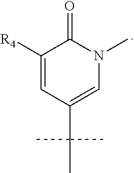

In a further embodiment, R₂ is hydrogen or C₁₋₆alkyl.

In a further embodiment, R₂ is heterocycloalkyl.

In a further embodiment, R₂ represents the group —CHR₅(CH₂)_{c}R₆.

In a further embodiment, R₅ is hydrogen.

In a further embodiment, R₅ is —(CH₂)_{d}OR₁₁.

In a further embodiment, R₆ is heterocycloalkyl.

In a further embodiment, R₆ is selected from the group consisting of:

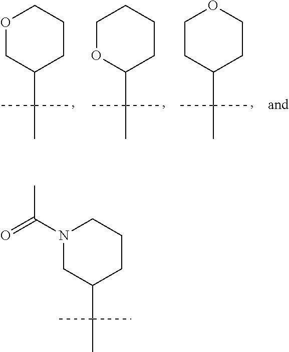

In a further embodiment, R₆ is

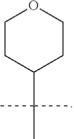

In a further embodiment, c is 0.

In a further embodiment, R₂ is selected from the group consisting of:

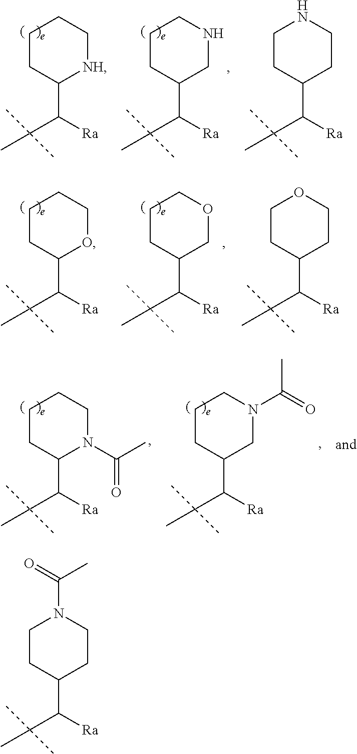

wherein Ra is hydrogen or C₁₋₃ alkyl; and e is 0 or 1.

In a further embodiment, R₂ is —CHR₅(CH₂)_{c}R₆, R₅ is —(CH₂)_{d}OR₁₁, b is 0 and R₆ is —(CH₂)_{d}OR₁₁.

In a further embodiment, both R₅ and R₆ represent —CH₂OCH₃.

In a further embodiment, R₄₃ is hydrogen, CH₃ or —OCH₃.

In a further embodiment, R₄₃ is CH₃ or —OCH₃.

In a further embodiment, R₄₃ is CH₃.

In a further embodiment, R_{4b} is C₁₋₃alkyl.

In a further embodiment, R_{4b} is CH₃.

In a further embodiment, b is 0.

In a further embodiment, R₄ₐ is hydrogen, CH₃ or —OCH₃, R_{4b} is CH₃ and b is 0.

In a further embodiment, a is 0.

In a further embodiment, a is 1 and R₃ is selected from the group consisting of halogen, —CN, C₁₋₃alkyl, and C-alkoxy-3alkoxy.

In a further embodiment, R₃ is halogen.

In a further embodiment, R₃ is chloro.

In a further embodiment, R₃ is at the 4-position on the imidazole ring.

In a further embodiment, a is 2 and each R₃ is independently selected from the group consisting of halogen, —CN, C₁₋₃alkyl, and C₁₋₃alkoxy.

In a further embodiment, each R₃ is independently selected from the group consisting of chloro, bromo, CH₃, and —CN.

In one embodiment, the present invention provides a compound of formula (I), excluding:
5-(1-(oxiran-2-ylmethyl)-1H-imidazol-5-yl)pyridin-2(1H)-one;
5-(1H-imidazol-2-yl)pyridin-2(1H)-one;
5-(4-hydroxy-1-methyl-1H-imidazol-2-yl)pyridin-2(1H)-one;
5-(5-(azetidin-3-yl)-1H-imidazol-1-yl)pyridin-2(1H)-one;
5-(5-hydroxy-1H-imidazol-2-yl)pyridin-2(1H)-one;
5-(5-hydroxy-4-methyl-1H-imidazol-2-yl)pyridin-2(1H)-one;
5-(1-ethyl-1H-imidazol-4-yl)-3-methylpyridin-2(1H)-one;
1-(6-oxo-1,6-dihydropyridin-3-yl)-1H-imidazole-4-carboxylic acid;
3-methyl-5-(1-methyl-1H-imidazol-4-yl)pyridin-2(1H)-one;
5-(1-ethyl-1H-imidazol-2-yl)-3-methylpyridin-2(1H)-one;
3-methyl-5-(1-propyl-1H-imidazol-2-yl)pyridin-2(1H)-one;
3-methyl-5-(1-methyl-1H-imidazol-2-yl)pyridin-2(1H)-one; and
5-(1-methyl-1H-imidazol-2-yl)pyridin-2(1H)-one.

In a further embodiment, the present invention provides compounds of formula (Ia), or salts thereof:

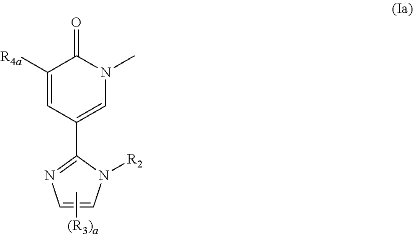

wherein
R₂ is C₁₋₆alkyl, C₁₋₆alkoxy, heterocycloalkyl or —CHR₅(CH₂)_{c}R₆;
each R₃ is independently selected from the group consisting of halogen, —CN, and C₁₋₃alkyl;
R₄ₐ is hydrogen, CH₃ or OCH₃;
R₅ is hydrogen, C₁₋₃alkyl, or —(CH₂)_{d}OR₁₁;
R₆ is hydrogen, C₁₋₃alkyl, —(CH₂)_{d}OR₁₁, or heterocycloalkyl, wherein the C₁₋₃alkyl, —(CH₂)_{d}OR₁₁, and heterocycloalkyl groups can be optionally substituted with one or two substituents independently selected from the group consisting of C₁₋₃alkyl, C₁₋₃alkoxy, halogen, —CH₂OH, —COOH, and —COCH₃;
R₁₁ is hydrogen or C₁₋₃alkyl;
a represents 0, 1 or 2;
c is 0 or 1; and
each d independently represents 0 or 1.

In one embodiment, the present invention provides a compound of formula (Ia):

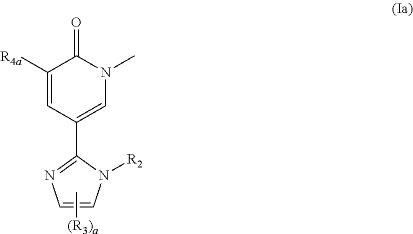

wherein
R₂ represents the group —CHR₅(CH₂)_{c}R₆;
each R₃ is independently selected from the group consisting of halogen, —CN, C₁₋₃alkyl, and C₁₋₃alkoxy;
R₄ₐ is CH₃ or —OCH₃;
R₅ is hydrogen or C₁₋₃alkyl;
R₆ is heterocycloalkyl;
a is 0, 1 or 2; and
c is 0 or 1.

In one embodiment, the present invention provides a compound of formula (Ia):

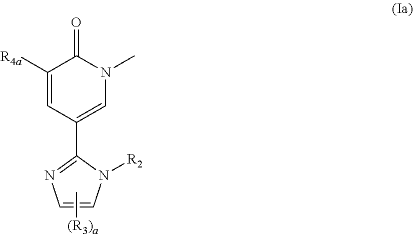

wherein
R₂ represents the group —CHR₅(CH₂)_{c}R₆;
each R₃ is independently selected from the group consisting of halogen, —CN, C₁₋₃alkyl, and C₁₋₃alkoxy;
R₄ₐ is CH₃ or —OCH₃;
R₅ is hydrogen or C₁₋₃alkyl;
R₆ is selected from the group consisting of

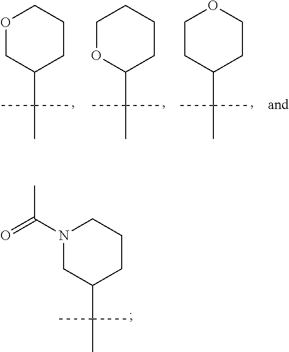

a is 0, 1 or 2; and
c is 0 or 1.

In one embodiment, the present invention provides a compound of formula (Ia):

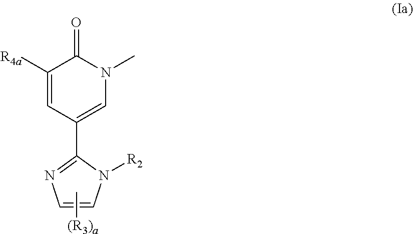

wherein
R₂ is selected from the group consisting of

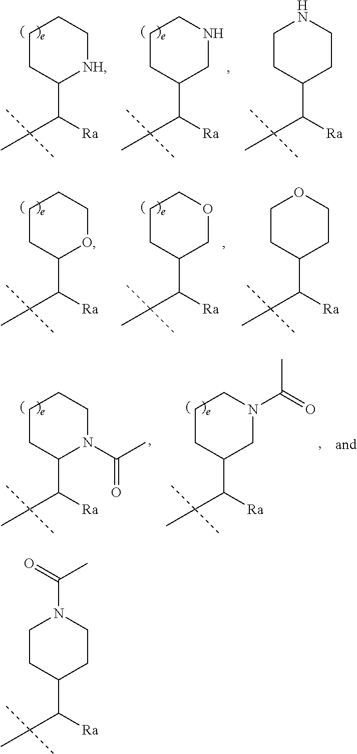

wherein Ra is hydrogen or C₁₋₃alkyl; and e is 0 or 1;
each R₃ is independently selected from the group consisting of halogen, —CN, C₁₋₃alkyl, and C₁₋₃alkoxy;
R₄ₐ is CH₃ or —OCH₃; and
a is 0, 1 or 2.

In one embodiment, the present invention provides a compound of formula (Ib):

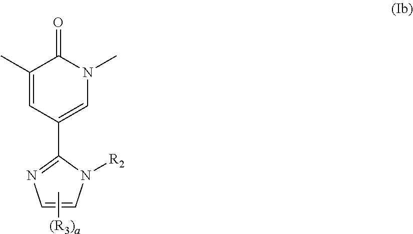

wherein
R₂ is selected from the group consisting of

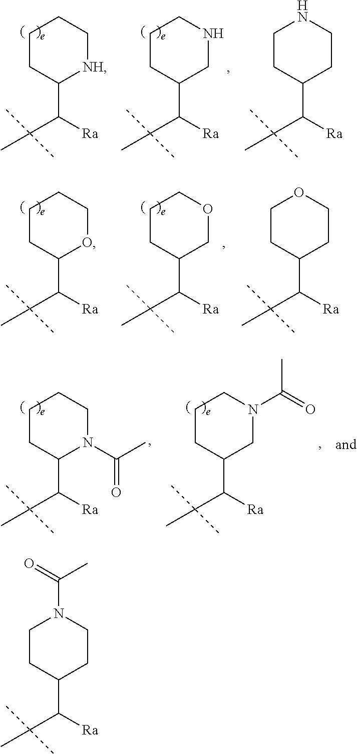

wherein Ra is hydrogen or C₁₋₃alkyl; and e is 0 or 1;
R₃ is selected from the group consisting of halogen, —CN, C₁₋₃alkyl, and C₁₋₃alkoxy; and
a is 1.

In one embodiment, the present invention provides a compound of formula (Ia):

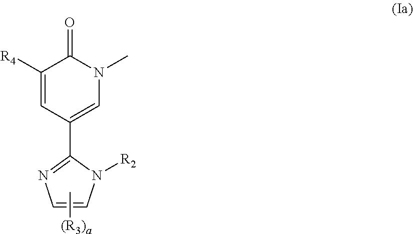

wherein
R₂ represents the group —CHR₅(CH₂)_{c}R₆;
each R₃ is independently selected from the group consisting of halogen, —CN, C₁₋₃alkyl, and C₁₋₃alkoxy;
R₄ is CH₃ or —OCH₃;
R₅ is —(CH₂)_{d}OR₁₁;
R₆ is —(CH₂)_{d}OR₁₁;
each R₁₁ independently represents C₁₋₃ alkyl;
a is 0, 1 or 2;
c is 0; and
d is 0 or 1.

In one embodiment, the present invention provides a compound of formula (Ia):

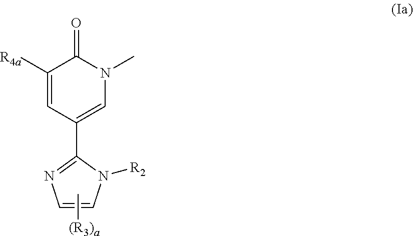

wherein
R₂ represents the group —CHR₅(CH₂)_{c}R₆, wherein both R₅ and R₆ represent —CH₂OCH₃;
each R₃ is independently selected from the group consisting of halogen, —CN, and C₁₋₃alkyl;
R₄ₐ is CH₃ or —OCH₃;
a is 0, 1 or 2; and
c is 0.

Specific examples of compounds of formula (I) are:
5-(1H-imidazol-2-yl)-1,3-dimethylpyridin-2(1H)-one;
5-(4-bromo-1-ethyl-1H-imidazol-2-yl)-1,3-dimethylpyridin-2(1H)-one;
5-(1-(cyclopropylmethyl)-1H-imidazol-2-yl)-1,3-dimethylpyridin-2(1H)-one;
5-(4-bromo-1-(cyclopropylmethyl)-1H-imidazol-2-yl)-1,3-dimethylpyridin-2(1H)-one;
5-(1-isobutyl-1H-imidazol-2-yl)-1,3-dimethylpyridin-2(1H)-one;
1,3-dimethyl-5-(1-((tetrahydro-2H-pyran-4-yl)methyl)-1H-imidazol-2-yl)pyridin-2(1H)-one;
1,3-dimethyl-5-(1-((tetrahydro-2H-pyran-2-yl)methyl)-1H-imidazol-2-yl)pyridin-2(1H)-one;
(R)-1,3-dimethyl-5-(1-((tetrahydro-2H-pyran-2-yl)methyl)-1H-imidazol-2-yl)pyridin-2(1H)-one;
(S)-1,3-dimethyl-5-(1-((tetrahydro-2H-pyran-2-yl)methyl)-1H-imidazol-2-yl)pyridin-2(1H)-one;
1,3-dimethyl-5-(1-(piperidin-4-ylmethyl)-1H-imidazol-2-yl)pyridin-2(1H)-one;
1,3-dimethyl-5-(1-((tetrahydrofuran-2-yl)methyl)-1H-imidazol-2-yl)pyridin-2(1H)-one;
5-(1-(2-methoxyethyl)-1H-imidazol-2-yl)-1,3-dimethylpyridin-2(1H)-one;
5-(1-(1,3-dimethoxypropan-2-yl)-1H-imidazol-2-yl)-1,3-dimethylpyridin-2(1H)-one;
methyl 2-(1,5-dimethyl-6-oxo-1,6-dihydropyridin-3-yl)-1H-imidazole-5-carboxylate;
5-(5-chloro-1H-imidazol-2-yl)-1,3-dimethylpyridin-2(1H)-one;
2-(1,5-dimethyl-6-oxo-1,6-dihydropyridin-3-yl)-1H-imidazole-5-carboxamide;
2-(1,5-dimethyl-6-oxo-1,6-dihydropyridin-3-yl)-1H-imidazole-4,5-dicarbonitrile;
5-(1-(1,3-dimethoxypropan-2-yl)-4,5-dimethyl-1H-imidazol-2-yl)-1,3-dimethylpyridin-2(1H)-one;
5-(4-(4-bromophenyl)-1-(1,3-dimethoxypropan-2-yl)-1H-imidazol-2-yl)-1,3-dimethylpyridin-2(1H)-one;
5-(4-chloro-1-((tetrahydro-2H-pyran-2-yl)methyl)-1H-imidazol-2-yl)-1,3-dimethylpyridin-2(1H)-one;
(R)-5-(4-chloro-1-((tetrahydro-2H-pyran-2-yl)methyl)-1H-imidazol-2-yl)-1,3-dimethylpyridin-2(1H)-one;
(S)-5-(4-chloro-1-((tetrahydro-2H-pyran-2-yl)methyl)-1H-imidazol-2-yl)-1,3-dimethylpyridin-2(1H)-one;
5-(5-chloro-1-((tetrahydro-2H-pyran-2-yl)methyl)-1H-imidazol-2-yl)-1,3-dimethylpyridin-2(1H)-one;
(R)-5-(5-chloro-1-((tetrahydro-2H-pyran-2-yl)methyl)-1H-imidazol-2-yl)-1,3-dimethylpyridin-2(1H)-one;
(S)-5-(5-chloro-1-((tetrahydro-2H-pyran-2-yl)methyl)-1H-imidazol-2-yl)-1,3-dimethylpyridin-2(1H)-one;
5-(5-chloro-1-((tetrahydro-2H-pyran-4-yl)methyl)-1H-imidazol-2-yl)-1,3-dimethylpyridin-2(1H)-one;
5-(5-chloro-1-((tetrahydro-2H-pyran-3-yl)methyl)-1H-imidazol-2-yl)-1,3-dimethylpyridin-2(1H)-one;
(R)-5-(5-chloro-1-((tetrahydro-2H-pyran-3-yl)methyl)-1H-imidazol-2-yl)-1,3-dimethylpyridin-2(1H)-one;
(S)-5-(5-chloro-1-((tetrahydro-2H-pyran-3-yl)methyl)-1H-imidazol-2-yl)-1,3-dimethylpyridin-2(1H)-one;
5-(4-chloro-1-(1,3-dimethoxypropan-2-yl)-1H-imidazol-2-yl)-1,3-dimethylpyridin-2(1H)-one;
5-(1-((1-acetylpiperidin-3-yl)methyl)-5-chloro-1H-imidazol-2-yl)-1,3-dimethylpyridin-2(1H)-one;
(R)-5-(1-((1-acetylpiperidin-3-yl)methyl)-5-chloro-1H-imidazol-2-yl)-1,3-dimethylpyridin-2(1H)-one;
(S)-5-(1-((1-acetylpiperidin-3-yl)methyl)-5-chloro-1H-imidazol-2-yl)-1,3-dimethylpyridin-2(1H)-one;
5-(1-((1-acetylpiperidin-3-yl)methyl)-4-chloro-1H-imidazol-2-yl)-1,3-dimethylpyridin-2(1H)-one;
(R)-5-(1-((1-acetylpiperidin-3-yl)methyl)-4-chloro-1H-imidazol-2-yl)-1,3-dimethylpyridin-2(1H)-one;
(S)-5-(1-((1-acetylpiperidin-3-yl)methyl)-4-chloro-1H-imidazol-2-yl)-1,3-dimethylpyridin-2(1H)-one;
5-(1-((1-acetylpiperidin-3-yl)methyl)-4-chloro-1H-imidazol-2-yl)-1,3-dimethylpyridin-2(1H)-one;
(R)-5-(1-((1-acetylpiperidin-3-yl)methyl)-4-chloro-1H-imidazol-2-yl)-1,3-dimethylpyridin-2(1H)-one
(S)-5-(1-((1-acetylpiperidin-3-yl)methyl)-4-chloro-1H-imidazol-2-yl)-1,3-dimethylpyridin-2(1H)-one
5-(4-chloro-1-((tetrahydro-2H-pyran-3-yl)methyl)-1H-imidazol-2-yl)-1,3-dimethylpyridin-2(1H)-one;
(R)-5-(4-chloro-1-((tetrahydro-2H-pyran-3-yl)methyl)-1H-imidazol-2-yl)-1,3-dimethylpyridin-2(1H)-one;
(S)-5-(4-chloro-1-((tetrahydro-2H-pyran-3-yl)methyl)-1H-imidazol-2-yl)-1,3-dimethylpyridin-2(1H)-one;
5-(4-chloro-1-((tetrahydro-2H-pyran-3-yl)methyl)-1H-imidazol-2-yl)-1,3-dimethylpyridin-2(1H)-one;
5-(4-chloro-1-((tetrahydro-2H-pyran-4-yl)methyl)-1H-imidazol-2-yl)-1,3-dimethylpyridin-2(1H)-one;
5-(1-ethyl-1H-imidazol-5-yl)-1,3-dimethylpyridin-2(1H)-one;
rac-1-(4-chloro-1-((tetrahydro-2H-pyran-3-yl)methyl)-1H-imidazol-2-yl)-3,5-dimethylpyridin-4(1H)-one;
methyl 2-(1,5-dimethyl-6-oxo-1,6-dihydropyridin-3-yl)-1-((tetrahydro-2H-pyran-4-yl)methyl)-1H-imidazole-4-carboxylate;
methyl 2-(1,5-dimethyl-6-oxo-1,6-dihydropyridin-3-yl)-1-((tetrahydro-2H-pyran-4-yl)methyl)-1H-imidazole-5-carboxylate;
2-(1,5-dimethyl-6-oxo-1,6-dihydropyridin-3-yl)-1-((tetrahydro-2H-pyran-4-yl)methyl)-1H-imidazole-4-carboxylic acid;
rac-5-(4-bromo-1-((tetrahydro-2H-pyran-3-yl)methyl)-1H-imidazol-2-yl)-1,3-dimethylpyridin-2(1H)-one;
rac-1-(4-bromo-1-((tetrahydro-2H-pyran-3-yl)methyl)-1H-imidazol-2-yl)-3,5-dimethylpyridin-4(1H)-one;
1-(4-bromo-1-((tetrahydro-2H-pyran-4-yl)methyl)-1H-imidazol-2-yl)-3,5-dimethylpyridin-4(1H)-one;
5-(4-bromo-1-((tetra hydro-2H-pyran-4-yl)methyl)-1H-imidazol-2-yl)-1,3-dimethylpyridin-2(1H)-one;
rac-5-(1-((1-acetylpiperidin-3-yl)methyl)-4-bromo-1H-imidazol-2-yl)-1,3-dimethylpyridin-2(1H)-one;
1-(4-chloro-1-(1,3-dimethoxypropan-2-yl)-1H-imidazol-2-yl)-3,5-dimethylpyridin-4(1H)-one; and
1-(4-chloro-1-((tetrahydro-2H-pyran-4-yl)methyl)-1H-imidazol-2-yl)-3,5-dimethylpyridin-4(1H)-one, or salts thereof.

In a further embodiment, the present invention provides a compound which is 5-(4-chloro-1-((tetrahydro-2H-pyran-4-yl)methyl)-1H-imidazol-2-yl)-1,3-dimethylpyridin-2(1H)-one, of formula:

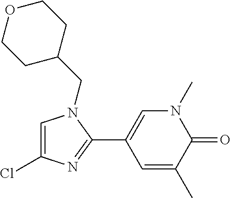 [SMILES: Cc1cc(-c2nc(Cl)cn2CC2CCOCC2)cn(C)c1=O]

or a salt thereof.

In a further embodiment, the present invention provides a compound which is 5-(4-chloro-1-(1,3-dimethoxypropan-2-yl)-1H-imidazol-2-yl)-1,3-dimethylpyridin-2(1H)-one, of formula:

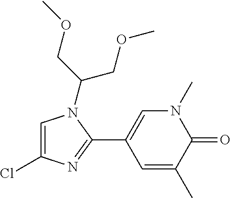 [SMILES: COCC(COC)n1cc(Cl)nc1-c1cc(C)c(=O)n(C)c1]

or a salt thereof.

In a further embodiment of the present invention, a compound of formula (I) is in the form of a free base. In one embodiment, the compound of formula (I) in the form of a free base is any one of the compounds of Examples 1 to 42.

Salts of the compounds of formula (I) include pharmaceutically acceptable salts and salts which may not be pharmaceutically acceptable but may be useful in the preparation of compounds of formula (I) and pharmaceutically acceptable salts thereof.

In one embodiment of the present invention, a compound of formula (I) is in the form of a pharmaceutically acceptable salt. In one embodiment, the compound of any of Example 1 to 42 is in the form of a pharmaceutically acceptable salt.

Compounds of formula (I) may contain an acidic or basic functional group and, thus, the skilled artisan will appreciate that pharmaceutically acceptable salts of the compounds of formula (I) may be prepared. Pharmaceutically acceptable salts of compounds of the invention may possess, for example, improved stability, solubility, and/or crystallinity, facilitating development as a medicine.

Compounds of formula (I) may contain a basic functional group and may be capable of forming pharmaceutically acceptable acid addition salts by treatment with an suitable acid (inorganic or organic acid). Representative pharmaceutically acceptable acid addition salts include hydrochloride, hydrobromide, nitrate, sulfate, bisulfate, sulfamate, phosphate, acetate, hydroxyacetate, phenylacetate, propionate, butyrate, isobutyrate, valerate, maleate, hydroxymaleate, acrylate, fumarate, maleate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, naphthoate, hydroxynaphthoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate. In another embodiment, the pharmaceutically acceptable salt is the 1,2-ethanedisulphonic acid (edisylate) salt.

Compounds of formula (I) may contain an acidic functional group and suitable pharmaceutically-acceptable salts include salts of such acidic functional groups. Representative salts include pharmaceutically acceptable metal salts such as sodium, potassium, lithium, calcium, magnesium, aluminum, and zinc salts; pharmaceutically acceptable organic primary, secondary, and tertiary amines including aliphatic amines, aromatic amines, aliphatic diamines, and hydroxy alkylamines such as methylamine, ethylamine, 2-hydroxyethylamine, diethylamine, TEA, ethylenediamine, ethanolamine, diethanolamine, and cyclohexylamine.

For a review on suitable salts see Berge et al., J. Pharm. Sci., 66:1-19 (1977). The invention includes within its scope all possible stoichiometric and non-stoichiometric forms of the salts of the compounds of formula (I).

Salts may be formed using techniques well-known in the art, for example by precipitation from solution followed by filtration, or by evaporation of the solvent.

It will be appreciated that many organic compounds can form complexes with solvents in which they are reacted or from which they are precipitated or crystallised. These complexes are known as “solvates”. For example, a complex with water is known as a “hydrate”. Solvents with high boiling points and/or solvents with a high propensity to form hydrogen bonds such as water, ethanol, iso-propyl alcohol, and N-methyl pyrrolidinone may be used to form solvates. Methods for the identification of solvates include, but are not limited to, NMR and microanalysis. Compounds of formula (I), or salts thereof, may exist in solvated and unsolvated form.

In one embodiment, there is provided a crystalline form of 5-(4-chloro-1-((tetrahydro-2H-pyran-4-yl)methyl)-1H-imidazol-2-yl)-1,3-dimethylpyridin-2(1H)-one monohydrate.

The crystalline hydrate has been characterised by X-ray powder diffraction (XRPD), Raman spectroscopy and thermogravimetric analysis (TGA).

### X-Ray Powder Diffraction (XRPD)

The data were acquired on PANalytical X'Pert Pro diffractometer using Ni-filtered Cu Ka (45 kV/40 mA) radiation and a step size of 0.02° 2θ and X'celerator™ RTMS (Real Time Multi-Strip) detector. Configuration on the incidental beam side: fixed divergence slit (0.250), 0.04 rad Soller slits, anti-scatter slit (0.250), and 10 mm beam mask. Configuration on the diffracted beam side: fixed divergence slit (0.250) and 0.04 rad Soller slit.

### FT-Raman Spectroscopy

Raman spectra were collected with a Nicolet NXR9650 or NXR 960 spectrometer (Thermo Electron) equipped with 1064 nm Nd:YVO₄ excitation laser, InGaAs and liquid-N₂ cooled Ge detectors, and a MicroStage. All spectra were acquired at 4 cm⁻¹ resolution, 64 scans, using Happ-Genzel apodization function and 2-level zero-filling through a glass cover.

### Thermogravimetric Analysis (TGA)

TGA thermograms were obtained with a TA Instruments Q500 thermogravimetric analyzer under 40 mL/min N₂ purge at 15° C./min in Al pans, unless otherwise noted.

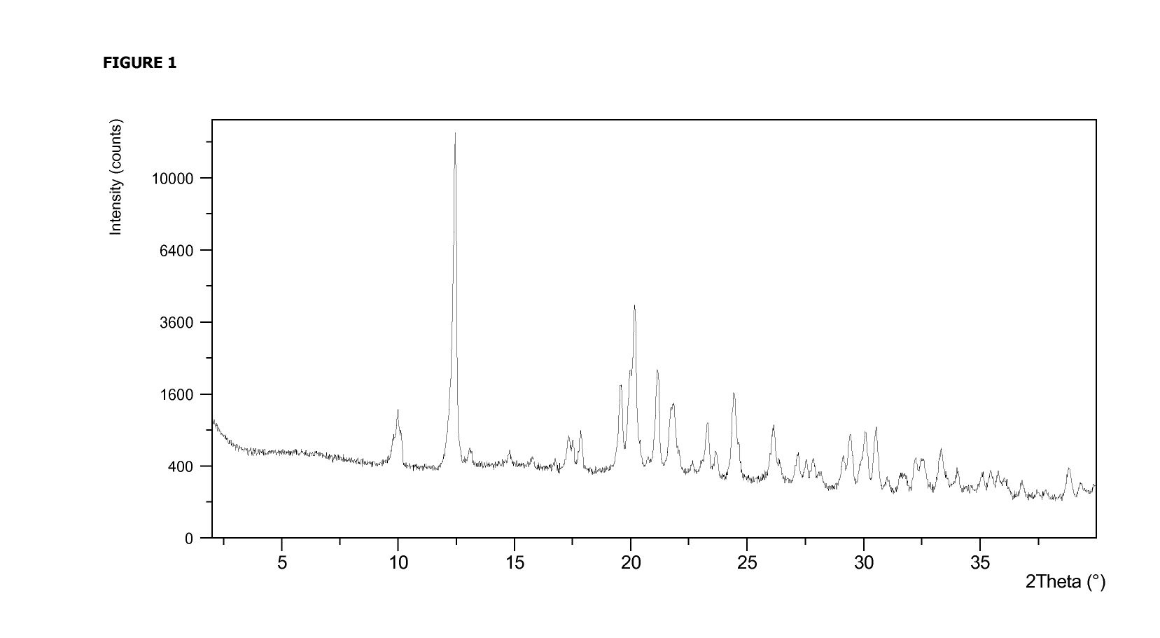

In a further embodiment, the crystalline form of 5-(4-chloro-1-((tetrahydro-2H-pyran-4-yl)methyl)-1H-imidazol-2-yl)-1,3-dimethylpyridin-2(1H)-one monohydrate has an X-ray powder diffraction pattern substantially as shown in FIG. 1.

Characteristic XRPD angles and d-spacings for Example 30a are recorded in Table 1. The margin of error is approximately +0.1° 2θ for each of the peak assignments. Peak intensities may vary from sample to sample due to preferred orientation. Peak positions were measured using PANalytical Highscore Plus software.

TABLE 1
Characteristic XRPD angles and d-spacings for Example 30a
Example 30a

| 2θ/° | d-spacings/Å |
|---|---|
| 10.0 | 8.9 |
| 12.4 | 7.1 |
| 13.1 | 6.8 |
| 14.8 | 6.0 |
| 15.8 | 5.6 |
| 17.9 | 5.0 |
| 19.6 | 4.5 |
| 20.2 | 4.4 |
| 21.2 | 4.2 |
| 23.3 | 3.8 |
| 24.4 | 3.6 |

In a further embodiment, there is provided a crystalline monohydrate form of 5-(4-chloro-1-((tetrahydro-2H-pyran-4-yl)methyl)-1H-imidazol-2-yl)-1,3-dimethylpyridin-2(1H)-one, which has an X-ray powder diffraction pattern with specific peaks at 2θ values, +0.1° 2θ experimental error, of 10.0, 12.4, 13.1, 14.8, 15.8, 17.9, 19.6, 20.2, 21.2, 23.3, and 24.4 degrees, as shown in Table 1.

In a further embodiment, there is provided a crystalline monohydrate form of 5-(4-chloro-1-((tetrahydro-2H-pyran-4-yl)methyl)-1H-imidazol-2-yl)-1,3-dimethylpyridin-2(1H)-one, which has an X-ray powder diffraction pattern with at least nine specific peaks at 2θ values, +0.1° 2θ experimental error, selected from a group consisting of 10.0, 12.4, 13.1, 14.8, 15.8, 17.9, 19.6, 20.2, 21.2, 23.3, and 24.4 degrees.

In a further embodiment, there is provided a crystalline monohydrate form of 5-(4-chloro-1-((tetrahydro-2H-pyran-4-yl)methyl)-1H-imidazol-2-yl)-1,3-dimethylpyridin-2(1H)-one, which has an X-ray powder diffraction pattern with at least eight or at least seven or at least six or at least five or at least four specific peaks at 2θ values, +0.1° 2θ experimental error, selected from a group consisting of 10.0, 12.4, 13.1, 14.8, 15.8, 17.9, 19.6, 20.2, 21.2, 23.3, and 24.4 degrees.

In a further embodiment, there is provided a crystalline monohydrate form of 5-(4-chloro-1-((tetrahydro-2H-pyran-4-yl)methyl)-1H-imidazol-2-yl)-1,3-dimethylpyridin-2(1H)-one, which has an X-ray powder diffraction pattern with at least three specific peaks at 2θ values, +0.1° 2θ experimental error, selected from a group consisting of 10.0, 12.4, 13.1, 14.8, 15.8, 17.9, 19.6, 20.2, 21.2, 23.3, and 24.4 degrees.

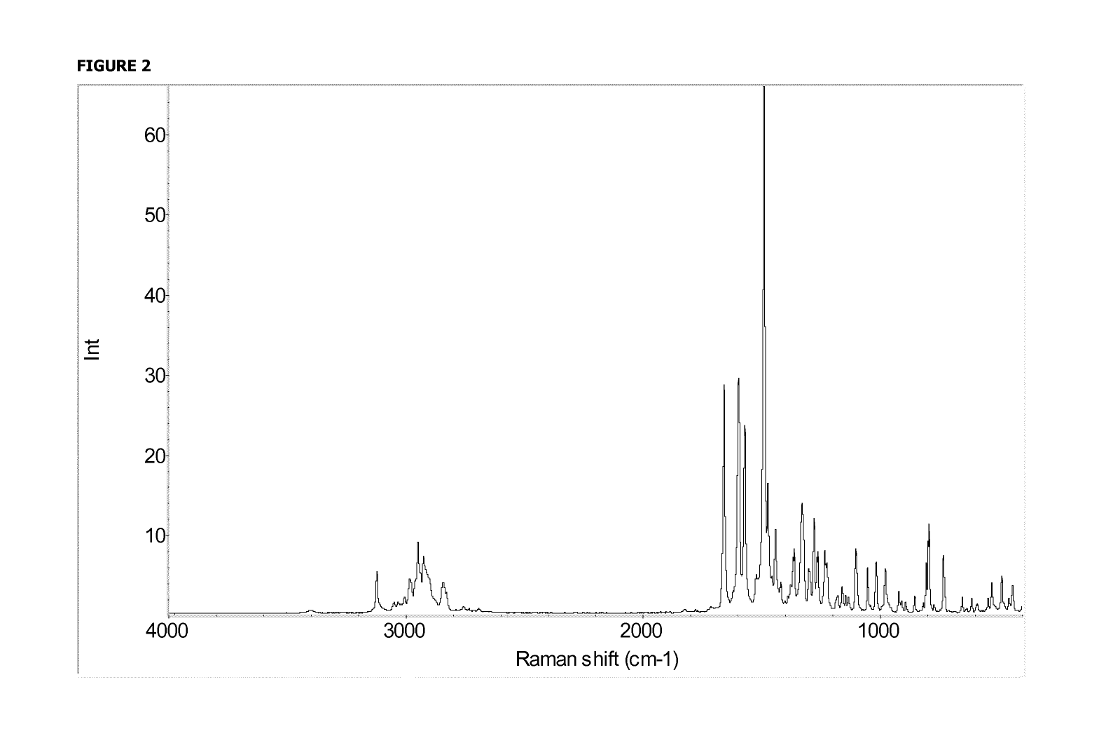

In a further embodiment, there is provided a crystalline monohydrate form of 5-(4-chloro-1-((tetrahydro-2H-pyran-4-yl)methyl)-1H-imidazol-2-yl)-1,3-dimethylpyridin-2(1H)-one, which has a FT Raman spectrum substantially as shown in FIG. 2.

In a further embodiment, there is provided a crystalline monohydrate form of 5-(4-chloro-1-((tetrahydro-2H-pyran-4-yl)methyl)-1H-imidazol-2-yl)-1,3-dimethylpyridin-2(1H)-one, characterised by an FT-Raman spectrum obtained under the conditions described hereinabove, comprising peaks at 440, 485, 528, 730, 794, 804, 919, 977, 1015, 1051, 1101, 1158, 1231, 1262, 1277, 1299, 1326, 1362, 1440, 1472, 1488, 1569, 1595, 1657, 2843, 2926, 2948, 3122 cm⁻¹, wherein the margin of error in each band position is approximately +1 cm⁻¹.

In a further embodiment, there is provided a crystalline monohydrate form of 5-(4-chloro-1-((tetrahydro-2H-pyran-4-yl)methyl)-1H-imidazol-2-yl)-1,3-dimethylpyridin-2(1H)-one, characterised by an FT-Raman spectrum obtained under the conditions described hereinabove, comprising at least eight peaks selected from a group consisting of 440, 485, 528, 730, 794, 804, 919, 977, 1015, 1051, 1101, 1158, 1231, 1262, 1277, 1299, 1326, 1362, 1440, 1472, 1488, 1569, 1595, 1657, 2843, 2926, 2948, 3122 cm⁻¹, wherein the margin of error in each band position is approximately +1 cm⁻¹.

In a further embodiment, there is provided a crystalline monohydrate form of 5-(4-chloro-1-((tetrahydro-2H-pyran-4-yl)methyl)-1H-imidazol-2-yl)-1,3-dimethylpyridin-2(1H)-one, characterised by an FT-Raman spectrum obtained under the conditions described hereinabove, comprising peaks of 977, 1595 and 1657 cm⁻¹, wherein the margin of error in each band position is approximately +1 cm⁻¹.

In a still further embodiment, there is provided a crystalline monohydrate form of 5-(4-chloro-1-((tetrahydro-2H-pyran-4-yl)methyl)-1H-imidazol-2-yl)-1,3-dimethylpyridin-2(1H)-one which, as a person having ordinary skill in the art will understand, is characterized by any combination of the analytical data characterizing the aforementioned embodiments.

In a further embodiment, there is provided a compound which has
a) an X-ray powder diffraction pattern (XRPD) substantially as shown in FIG. 1; and/or
b) an X-ray powder diffraction pattern (XRPD) with specific peaks at 2θ values, +0.1° 2θ experimental error, of 10.0, 12.4, 13.1, 14.8, 15.8, 17.9, 19.6, 20.2, 21.2, 23.3, and 24.4 degrees; and/or
(c) a FT Raman spectrum substantially as shown in FIG. 2.

It is well known and understood to those skilled in the art that the apparatus employed, humidity, temperature, orientation of the powder crystals, and other parameters involved in obtaining an X-ray powder diffraction (XRPD) pattern may cause some variability in the appearance, intensities, and positions of the lines in the diffraction pattern. An X-ray powder diffraction pattern that is “substantially as shown in FIG. 1” provided herein is an XRPD pattern that would be considered by one skilled in the art to represent a compound possessing the same crystal form as the compound that provided the XRPD pattern of FIG. 1. That is, the XRPD pattern may be identical to that of FIG. 1, or more likely it may be somewhat different. Such an XRPD pattern may not necessarily show each of the lines of any one of the diffraction patterns presented herein, and/or may show a slight change in appearance, intensity, or a shift in position of said lines resulting from differences in the conditions involved in obtaining the data. A person skilled in the art is capable of determining if a sample of a crystalline compound has the same form as, or a different form from, a form disclosed herein by comparison of their XRPD patterns. For example, one skilled in the art can overlay an XRPD pattern of a sample of a crystalline monohydrate form of 5-(4-chloro-1-((tetrahydro-2H-pyran-4-yl)methyl)-1H-imidazol-2-yl)-1,3-dimethylpyridin-2(1H)-one, with FIG. 1 and, using expertise and knowledge in the art, readily determine whether the XRPD pattern of the sample is substantially as shown in FIG. 1. If the XRPD pattern is substantially as shown in FIG. 1, the sample form can be readily and accurately identified as having the same form as the compound of the invention.

Further, it is also well known and understood to those skilled in the art that the apparatus employed, humidity, temperature, orientation of the powder crystals, and other parameters involved in obtaining a Raman spectrum may cause some variability in the appearance, intensities, and positions of the peaks in the spectrum. A Raman spectrum that is “substantially as shown in FIG. 2” provided herein is a Raman spectrum that would be considered by one skilled in the art to represent a compound possessing the same crystal form as the compound that provided the Raman spectrum of FIG. 2. That is, the Raman spectrum may be identical to that of FIG. 2, or more likely it may be somewhat different. Such a Raman spectrum may not necessarily show each of the peaks of any one of the spectra presented herein, and/or may show a slight change in appearance, intensity, or a shift in position of said peaks resulting from differences in the conditions involved in obtaining the data. A person skilled in the art is capable of determining if a sample of a crystalline compound has the same form as, or a different form from, a form disclosed herein by comparison of their Raman spectra. For example, one skilled in the art can overlay a Raman spectrum of a sample of a crystalline monohydrate form of 5-(4-chloro-1-((tetrahydro-2H-pyran-4-yl)methyl)-1H-imidazol-2-yl)-1,3-dimethylpyridin-2(1H)-one, with FIG. 2 and, using expertise and knowledge in the art, readily determine whether the Raman spectrum of the sample is substantially as shown in FIG. 2. If the XRPD pattern is substantially as shown in FIG. 1, the sample form can be readily and accurately identified as having the same form as the compound of the invention.

In a preferred embodiment, the hydrate is in crystalline form. Amorphous forms of the hydrate (e.g. amorphous monohydrate) also form part of the present invention. For a crystalline hydrated form, the degree of crystallinity is greater than about 60%, 70%, 80%, 90%, 95%, 96%, 97%, 98%, or 99%. In one embodiment, the degree of crystallinity is greater than 99%.

Certain of the compounds of the invention may exist in tautomeric forms. It will be understood that the present invention encompasses all of the tautomers of the compounds of the invention whether as individual tautomers or as mixtures thereof.

The compounds of the invention may be in crystalline or amorphous form. The most thermodynamically stable crystalline form of a compound of the invention is of particular interest.

Crystalline forms of compounds of the invention may be characterised and differentiated using a number of conventional analytical techniques, including, but not limited to, X-ray powder diffraction (XRPD), infrared spectroscopy (IR), Raman spectroscopy, differential scanning calorimetry (DSC), thermogravimetric analysis (TGA) and solid-state nuclear magnetic resonance (ssNMR).

The present invention also includes all suitable isotopic variations of a compound of formula (I) or a pharmaceutically acceptable salt thereof. An isotopic variation of a compound of formula (I), or a pharmaceutically acceptable salt thereof, is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, fluorine and chlorine such as ²H, ³H, ¹³C, ¹⁴C, ¹⁵N, ¹⁷O, ¹⁸O, ¹⁸F and ³⁶Cl, respectively. Certain isotopic variations of a compound of formula (I) or a salt or solvate thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of a compound of formula (I), or a pharmaceutically salt thereof, can generally be prepared by conventional procedures such as by the illustrative methods or by the preparations described in the Examples hereafter using appropriate isotopic variations of suitable reagents.

The compounds of formula (I) and pharmaceutically acceptable salts thereof may contain one or more asymmetric center (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in a compound of formula (I), or in any chemical structure illustrated herein, is not specified the structure is intended to encompass all individual stereoisomers and all mixtures thereof. Thus, compounds of formula (I) and pharmaceutically acceptable salts thereof containing one or more chiral center may be used as racemic mixtures, enantiomerically enriched mixtures, or as enantiomerically pure individual stereoisomers.

Individual stereoisomers of a compound of formula (I), or a pharmaceutically acceptable salt thereof, which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

In one embodiment, a compound of the invention is capable of inhibiting the binding of one or more of the four known BET family bromodomain containing proteins (e.g. BRD2, BRD3, BRD4 and BRDt) to, for example, an acetylated lysine residue. In a further embodiment, the compound of formula (I), or a pharmaceutically acceptable salt thereof, is capable of inhibiting the binding of BRD4 to its cognate acetylated lysine residue. The compounds of the invention may possess an improved profile over known BET inhibitors, for example, certain compounds may have one or more of the following properties:
(i) potent BET inhibitory activity;
(ii) selectivity over other known bromodomain containing proteins outside of the BET family of proteins;
(iii) selectivity for a particular BET family member over other BET family members;
(iv) selectivity for one Binding Domain (i.e. BD1 over BD2 or vice versa) for any given BET family member;
(v) improved developability (e.g. desirable solubility profile, pharmacokinetics and pharmacodynamics); or
(vi) a reduced side-effect profile.

### Statement of Use

Compounds of formula (I), or pharmaceutically acceptable salts thereof, are BET inhibitors and thus may have therapeutic utility in the treatment of a variety of diseases or conditions related to systemic or tissue inflammation, inflammatory responses to infection or hypoxia, cellular activation and proliferation, lipid metabolism, fibrosis and in the prevention and treatment of viral infections.

BET inhibitors may be useful in the treatment of a wide variety of acute or chronic autoimmune or inflammatory conditions such as rheumatoid arthritis, osteoarthritis, acute gout, psoriasis, psoriatic arthritis, spondyloarthritis, systemic lupus erythematosus, pulmonary arterial hypertension (PAH), multiple sclerosis, inflammatory bowel disease (Crohn's disease and ulcerative colitis), asthma, chronic obstructive airways disease, pneumonitis, myocarditis, pericarditis, myositis, eczema, dermatitis (including atopic dermatitis), alopecia, vitiligo, bullous skin diseases, nephritis, vasculitis, hypercholesterolemia, atherosclerosis, Alzheimer's disease, depression, Sjögren's syndrome, sialoadenitis, central retinal vein occlusion, branched retinal vein occlusion, Irvine-Gass syndrome (post cataract and post-surgical), retinitis pigmentosa, pars planitis, birdshot retinochoroidopathy, epiretinal membrane, cystic macular edema, parafoveal telengiectasis, tractional maculopathies, vitreomacular traction syndromes, retinal detachment, neuroretinitis, idiopathic macular edema, retinitis, dry eye (keratoconjunctivitis Sicca), vernal keratoconjunctivitis, atopic keratoconjunctivitis, uveitis (such as anterior uveitis, pan uveitis, posterior uveitis, uveitis-associated macular edema), scleritis, diabetic retinopathy, diabetic macular edema, age-related macular dystrophy, hepatitis, pancreatitis, primary biliary cirrhosis, sclerosing cholangitis, acute alcoholic hepatitis, chronic alcoholic hepatitis, alcoholic steato-hepatitis, non-alcoholic steato-hepatitis (NASH), cirrhosis, Childs-Pugh cirrhosis, autoimmune hepatitis, fulminant hepatitis, chronic viral hepatitis, alcoholic liver disease, systemic sclerosis, systemic sclerosis with associated interstitial lung disease, sarcoidosis, neurosarcoidosis, Addison's disease, hypophysitis, thyroiditis, type I diabetes, giant cell arteritis, nephritis including lupus nephritis, vasculitis with organ involvement such as glomerulonephritis, vasculitis including giant cell arteritis, Wegener's granulomatosis, Polyarteritis nodosa, Behcet's disease, Kawasaki disease, Takayasu's arteritis, pyoderma gangrenosum, vasculitis with organ involvement, chronic organ transplant rejection and acute rejection of transplanted organs. The use of BET inhibitors for the treatment of rheumatoid arthritis and NASH are of particular interest.

In one embodiment, the acute or chronic autoimmune or inflammatory condition is a disorder of lipid metabolism via the regulation of APO-A1 such as hypercholesterolemia, atherosclerosis and Alzheimer's disease.

In another embodiment, the acute or chronic autoimmune or inflammatory condition is a respiratory disorder such as asthma or chronic obstructive airways disease.

In another embodiment, the acute or chronic autoimmune or inflammatory condition is a systemic inflammatory disorder such as rheumatoid arthritis, osteoarthritis, acute gout, psoriasis, systemic lupus erythematosus, multiple sclerosis or inflammatory bowel disease (Crohn's disease and ulcerative colitis).

In another embodiment, the acute or chronic autoimmune or inflammatory condition is multiple sclerosis.

In a further embodiment, the acute or chronic autoimmune or inflammatory condition is type I diabetes.

BET inhibitors may be useful in the treatment of diseases or conditions which involve inflammatory responses to infections with bacteria, viruses, fungi, parasites or their toxins, such as sepsis, acute sepsis, sepsis syndrome, septic shock, endotoxaemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, post-surgical syndromes, sarcoidosis, Herxheimer reactions, encephalitis, myelitis, meningitis, malaria and SIRS associated with viral infections such as influenza, herpes zoster, herpes simplex and coronavirus. In one embodiment, the disease or condition which involves an inflammatory response to an infection with bacteria, a virus, fungi, a parasite or their toxins is acute sepsis.

BET inhibitors may be useful in the treatment of conditions associated with ischaemia-reperfusion injury such as myocardial infarction, cerebro-vascular ischaemia (stroke), acute coronary syndromes, renal reperfusion injury, organ transplantation, coronary artery bypass grafting, cardio-pulmonary bypass procedures, pulmonary, renal, hepatic, gastro-intestinal or peripheral limb embolism.

BET inhibitors may be useful in the treatment of fibrotic conditions such as idiopathic pulmonary fibrosis, renal fibrosis, liver fibrosis, post-operative stricture, keloid scar formation, scleroderma (including morphea), cardiac fibrosis and cystic fibrosis.

BET inhibitors may be useful in the treatment of viral infections such as herpes simplex infections and reactivations, cold sores, herpes zoster infections and reactivations, chickenpox, shingles, human papilloma virus (HPV), human immunodeficiency virus (HIV), cervical neoplasia, adenovirus infections, including acute respiratory disease, poxvirus infections such as cowpox and smallpox and African swine fever virus. In one embodiment, the viral infection is a HPV infection of skin or cervical epithelia. In another embodiment, the viral infection is a latent HIV infection.

BET inhibitors may be useful in the treatment of cancer, including hematological (such as leukaemia, lymphoma and multiple myeloma), epithelial including lung, breast and colon carcinomas, midline carcinomas, mesenchymal, hepatic, renal and neurological tumours.

BET inhibitors may be useful in the treatment of one or more cancers selected from brain cancer (gliomas), glioblastomas, Bannayan-Zonana syndrome, Cowden disease, Lhermitte-Duclos disease, breast cancer, inflammatory breast cancer, colorectal cancer, Wilm's tumor, Ewing's sarcoma, rhabdomyosarcoma, ependymoma, medulloblastoma, colon cancer, head and neck cancer, kidney cancer, lung cancer, liver cancer, melanoma, squamous cell carcinoma, ovarian cancer, pancreatic cancer, prostate cancer, sarcoma cancer, osteosarcoma, giant cell tumor of bone, thyroid cancer, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma, megakaryoblastic leukemia, acute megakaryocytic leukemia, promyelocytic leukemia, mixed lineage leukaemia, erythroleukemia, malignant lymphoma, Hodgkins lymphoma, non-Hodgkins lymphoma, lymphoblastic T-cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), NUT-midline carcinoma and testicular cancer.

In one embodiment, the cancer is a leukaemia, for example a leukaemia selected from acute monocytic leukemia, acute myelogenous leukemia, chronic myelogenous leukemia, chronic lymphocytic leukemia and mixed lineage leukaemia (MLL). In another embodiment, the cancer is NUT-midline carcinoma. In another embodiment, the cancer is multiple myeloma. In another embodiment, the cancer is a lung cancer such as small cell lung cancer (SCLC). In another embodimnet, the cancer is a neuroblastoma. In another embodiment, the cancer is Burkitt's lymphoma. In another embodiment, the cancer is cervical cancer. In another embodiment, the cancer is esophageal cancer. In another embodiment, the cancer is ovarian cancer. In another embodiment, the cancer is breast cancer. In another embodiment, the cancer is colorectal cancer. In another embodiment, the cancer is prostate cancer. In another embodiment, the cancer is castration-resistant prostate cancer.

In one embodiment, the disease or condition for which a BET inhibitor is indicated is selected from diseases associated with systemic inflammatory response syndrome, such as sepsis, burns, pancreatitis, major trauma, haemorrhage and ischaemia. In this embodiment, the BET inhibitor would be administered at the point of diagnosis to reduce the incidence of SIRS, the onset of shock, multi-organ dysfunction syndrome, which includes the onset of acute lung injury, ARDS, acute renal, hepatic, cardiac or gastro-intestinal injury and mortality. In another embodiment, the BET inhibitor would be administered prior to surgical or other procedures associated with a high risk of sepsis, haemorrhage, extensive tissue damage, SIRS or MODS (multiple organ dysfunction syndrome). In a particular embodiment, the disease or condition for which a BET inhibitor is indicated is sepsis, sepsis syndrome, septic shock and endotoxaemia. In another embodiment, the BET inhibitor is indicated for the treatment of acute or chronic pancreatitis. In another embodiment, the BET inhibitor is indicated for the treatment of burns.

In a further aspect, the present invention also provides a compound of formula (I) or a pharmaceutically acceptable salt thereof for use in therapy.

In one embodiment, the present invention provides 5-(4-chloro-1-((tetrahydro-2H-pyran-4-yl)methyl)-1H-imidazol-2-yl)-1,3-dimethylpyridin-2(1H)-one, of formula:

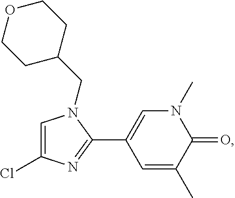 [SMILES: Cc1cc(-c2nc(Cl)cn2CC2CCOCC2)cn(C)c1=O]

or a pharmaceutically acceptable salt thereof, for use in therapy.

In a further embodiment, the present invention provides 5-(4-chloro-1-(1,3-dimethoxypropan-2-yl)-1H-imidazol-2-yl)-1,3-dimethylpyridin-2(1H)-one, of formula:

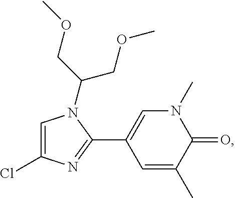 [SMILES: COCC(COC)n1cc(Cl)nc1-c1cc(C)c(=O)n(C)c1]

or a pharmaceutically acceptable salt thereof, for use in therapy.

In one embodiment, the present invention provides 5-(4-chloro-1-((tetrahydro-2H-pyran-4-yl)methyl)-1H-imidazol-2-yl)-1,3-dimethylpyridin-2(1H)-one monohydrate, of formula:

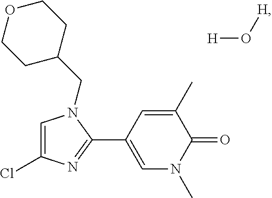

for use in therapy.

In a further aspect, the present invention provides a compound of formula (I) or a pharmaceutically acceptable salt thereof for use in the treatment of diseases or conditions for which a bromodomain inhibitor, in particular a BET inhibitor, is indicated, including each and all of the above listed indications.

In a further aspect, the present invention also provides a compound of formula (I), or a pharmaceutically acceptable salt thereof, for use in the treatment of autoimmune and inflammatory diseases, and cancer.

In a further aspect, the present invention provides a compound of formula (I), or a pharmaceutically acceptable salt thereof, for use in the treatment of rheumatoid arthritis. In a further aspect, the present invention provides a compound of formula (I), or a pharmaceutically acceptable salt thereof, for use in the treatment of therapy-resistant rheumatoid arthritis.

In a further aspect, the present invention is directed to a method of treatment of an autoimmune or inflammatory disease or cancer, which comprises administering to a subject in need thereof, a therapeutically effective amount of a compound of formula (I), or a pharmaceutically acceptable salt thereof.

In yet a further aspect, the present invention is directed to a method of treating rheumatoid arthritis, which comprises administering to a subject in need thereof, a therapeutically effective amount of a compound of formula (I), or a pharmaceutically acceptable salt thereof.

In a further aspect, the present invention is directed to the use of a compound of formula (I), or a pharmaceutically acceptable salt thereof, in the manufacture of a medicament for use in the treatment of an autoimmune or inflammatory disease, or cancer.

In a further aspect, the present invention is directed to the use of a compound of formula (I), or a pharmaceutically acceptable salt thereof, in the manufacture of a medicament for use in the treatment of rheumatoid arthritis.

### Pharmaceutical Compositions/Routes of Administration/Dosages

While it is possible that for use in therapy, a compound of formula (I) as well as pharmaceutically acceptable salts thereof may be administered as the raw chemical, it is common to present the active ingredient as a pharmaceutical composition.

In a further aspect, there is provided a pharmaceutical composition comprising a compound of formula (I), or a pharmaceutically acceptable salt thereof, and one or more pharmaceutically acceptable excipients. In a further aspect, there is provided a pharmaceutical composition comprising a compound of formula (I), or a pharmaceutically acceptable salt thereof, and a pharmaceutically acceptable excipient.

In a further embodiment, there is provided a pharmaceutical composition comprising 5-(4-chloro-1-((tetrahydro-2H-pyran-4-yl)methyl)-1H-imidazol-2-yl)-1,3-dimethylpyridin-2(1H)-one, of formula:

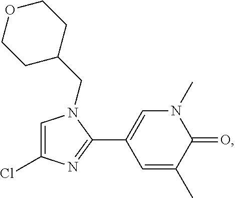 [SMILES: Cc1cc(-c2nc(Cl)cn2CC2CCOCC2)cn(C)c1=O]

or a pharmaceutically acceptable salt thereof, and one or more pharmaceutically acceptable excipients.

In a further aspect, there is provided a pharmaceutical composition comprising 5-(4-chloro-1-(1,3-dimethoxypropan-2-yl)-1H-imidazol-2-yl)-1,3-dimethylpyridin-2(1H)-one, of formula:

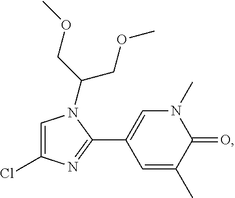 [SMILES: COCC(COC)n1cc(Cl)nc1-c1cc(C)c(=O)n(C)c1]

or a pharmaceutically acceptable salt thereof, and one or more pharmaceutically acceptable excipients.

In a further embodiment, there is provided a pharmaceutical composition comprising 5-(4-chloro-1-((tetrahydro-2H-pyran-4-yl)methyl)-1H-imidazol-2-yl)-1,3-dimethylpyridin-2(1H)-one monohydrate, of formula:

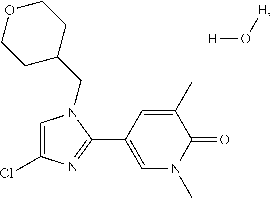

and one or more pharmaceutically acceptable excipients.

The excipient(s) must be pharmaceutically acceptable and be compatible with the other ingredients of the composition. In accordance with another aspect of the invention there is also provided a process for the preparation of a pharmaceutical composition including admixing a compound of formula (I), or a pharmaceutically acceptable salt thereof, with one or more pharmaceutically acceptable excipients. The pharmaceutical composition can be used in the treatment of any of the diseases described herein.

Since the compounds of formula (I) are intended for use in pharmaceutical compositions it will be readily understood that they are each preferably provided in substantially pure form, for example, at least 85% pure, especially at least 98% pure (% in a weight for weight basis).

Pharmaceutical compositions may be presented in unit dose forms containing a predetermined amount of active ingredient per unit dose. Preferred unit dosage compositions are those containing a daily dose or sub-dose, or an appropriate fraction thereof, of an active ingredient. Such unit doses may therefore be administered more than once a day.

Pharmaceutical compositions may be adapted for administration by any appropriate route, for example by the oral (including buccal or sublingual), rectal, inhaled, intranasal, topical (including buccal, sublingual or transdermal), ocular (including topical, intraocular, subconjunctival, episcleral, sub-Tenon), vaginal or parenteral (including subcutaneous, intramuscular, intravenous or intradermal) route. Such compositions may be prepared by any method known in the art of pharmacy, for example by bringing into association the active ingredient with the excipient(s).

Compounds of the invention, in particular, 5-(4-chloro-1-((tetrahydro-2H-pyran-4-yl)methyl)-1H-imidazol-2-yl)-1,3-dimethylpyridin-2(1H)-one and hydrated (e.g. monohydrate) versions thereof, may possess a pK profile that is supportive of both oral and IV infusion, for example, once-daily in humans.

In one aspect, the pharmaceutical composition is adapted for oral administration.

In a further aspect, the pharmaceutical composition is adapted for intravenous administration.

Pharmaceutical compositions adapted for oral administration may be presented as discrete units such as tablets or capsules; powders or granules; solutions or suspensions in aqueous or non-aqueous liquids; edible foams or whips; or oil-in-water liquid emulsions or water-in-oil liquid emulsions.

Powders suitable for incorporating into tablets or capsules may be prepared by reducing the compound to a suitable fine size (e.g. by micronisation) and mixing with a similarly prepared pharmaceutical excipient such as an edible carbohydrate, for example, starch or mannitol. Flavoring, preservative, dispersing and coloring agents, for example, may also be present.

Capsules may be made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, glidants, lubricants, sweetening agents, flavours, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of formula (I) and pharmaceutically acceptable salts thereof can also be combined with a free flowing inert excipient and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Compositions for oral administration may be designed to provide a modified release profile so as to sustain or otherwise control the release of the therapeutically active agent.

Where appropriate, dosage unit compositions for oral administration can be microencapsulated. The composition may be prepared to prolong or sustain the release as for example by coating or embedding particulate material in polymers, wax or the like.

Pharmaceutical compositions for nasal or inhaled administration may conveniently be formulated as aerosols, solutions, suspensions, gels or dry powders.

For pharmaceutical compositions suitable for and/or adapted for inhaled administration, it is preferred that a compound of formula (I) or a pharmaceutically acceptable salt thereof, is in a particle-size-reduced form e.g. obtained by micronisation. The preferable particle size of the size-reduced (e.g. micronised) compound or salt is defined by a D50 value of about 0.5 to about 10 microns (for example as measured using laser diffraction).

For pharmaceutical compositions suitable for and/or adapted for inhaled administration, the pharmaceutical composition may be a dry powder composition or an aerosol formulation, comprising a solution or fine suspension of the active substance in a pharmaceutically acceptable aqueous or non-aqueous solvent. Dry powder compositions can comprise a powder base such as lactose, glucose, trehalose, mannitol or starch, the compounds of formula (I) or a pharmaceutically acceptable salt thereof (preferably in particle-size-reduced form, e.g. in micronised form), and optionally a performance modifier such as L-leucine or another amino acid and/or metal salt of stearic acid such as magnesium or calcium stearate. Preferably, the dry powder inhalable composition comprises a dry powder blend of lactose e.g. lactose monohydrate and the compound of formula (I) or a salt thereof.

In one embodiment, a dry powder composition suitable for inhaled administration may be incorporated into a plurality of sealed dose containers provided on medicament pack(s) mounted inside a suitable inhalation device. The containers may be rupturable, peelable or otherwise openable one-at-a-time and the doses of the dry powder composition administered by inhalation on a mouthpiece of the inhalation device, as known in the art. The medicament pack may take a number of different forms, for instance a disk-shape or an elongate strip. Representative inhalation devices are the DISKHALER™ inhaler device, the DISKUS™ inhalation device, and the ELLIPTA™ inhalation device, marketed by GlaxoSmithKline. The DISKUS™ inhalation device is, for example, described in GB 2242134A, and the ELLIPTA™ inhalation device is, for example, described in WO 2003/061743 A1, WO 2007/012871 A1 and/or WO 2007/068896 A1.

Pharmaceutical compositions adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Pharmaceutical compositions adapted for topical administration may be formulated as ointments, creams, suspensions, emulsions, lotions, powders, solutions, pastes, gels, foams, sprays, aerosols or oils. Such pharmaceutical compositions may include conventional additives which include, but are not limited to, preservatives, solvents to assist drug penetration, co-solvents, emollients, propellants, viscosity modifying agents (gelling agents), surfactants and carriers. In one embodiment there is provided a pharmaceutical composition adapted for topical administration which comprises between 0.01-10%, or between 0.01-1% of a compound of formula (I)-(XVI), or a pharmaceutically acceptable salt thereof, by weight of the composition.

For treatments of the eye or other external tissues, for example mouth and skin, the compositions are preferably applied as a topical ointment, cream, gel, spray or foam. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administrations to the eye include eye drops wherein the active ingredient is dissolved or suspended in a suitable carrier, especially an aqueous solvent.

A therapeutically effective amount of a compound of formula (I) or a pharmaceutically acceptable salt thereof, will depend upon a number of factors including, for example, the age and weight of the subject, the precise condition requiring treatment and its severity, the nature of the formulation, and the route of administration, and will ultimately be at the discretion of the attendant physician or veterinarian. In the pharmaceutical composition, each dosage unit for oral administration preferably contains from 0.01 to 1000 mg, more preferably 0.5 to 100 mg, of a compound of formula (I) or a pharmaceutically acceptable salt thereof, calculated as the free base. In one embodiment, the compound of the invention is administered orally at a daily dose of 0.5 to 20 mg, for example 10 to 20 mg. In a further embodiment, a compound of the invention is administered intraveniously at a daily dose of 0.5 to 10 mg, for example 5 to 10 mg.

The compounds of formula (I) and pharmaceutically acceptable salts thereof may be employed alone or in combination with other therapeutic agents. Combination therapies according to the present invention thus comprise the administration of at least one compound of formula (I) or a pharmaceutically acceptable salt thereof, and the use of at least one other therapeutically active agent. A compound of formula (I) or pharmaceutically acceptable salt thereof, and the other therapeutically active agent(s) may be administered together in a single pharmaceutical composition or separately and, when administered separately this may occur simultaneously or sequentially in any order.

In a further aspect, there is provided a combination product comprising a compound of formula (I) or a pharmaceutically acceptable salt thereof, together with one or more other therapeutically active agents, and optionally one or more pharmaceutically acceptable excipients.

It will be clear to a person skilled in the art that, where appropriate, the other therapeutic ingredient(s) may be used in the form of salts, for example as alkali metal or amine salts or as acid addition salts, or as solvates, for example hydrates, to optimise the activity and/or stability and/or physical characteristics, such as solubility, of the therapeutic ingredient. It will be clear also that, where appropriate, the therapeutic ingredients may be used in optically pure form.

The combinations referred to above may conveniently be presented for use in the form of a pharmaceutical composition and thus pharmaceutical compositions comprising a combination as defined above together with a pharmaceutically acceptable excipient.

### General Synthetic Routes

Compounds of formula (I) and salts thereof may be prepared by the methodology described hereinafter, constituting further aspects of this invention.

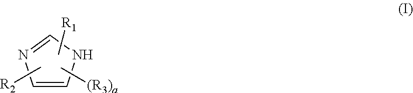

wherein R₁, R₂, R₃ and a are as defined hereinbefore for a compound of formula (I).

Accordingly, there is provided a process for the preparation of a compound of formula (IIa):

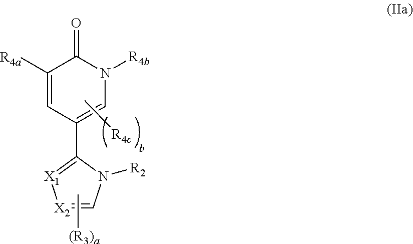

which process comprises the alkylation of a compound of formula (III):

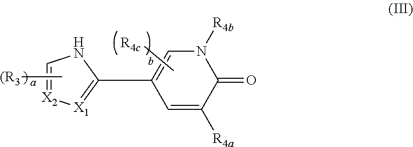

wherein R₃, R₄ₐ, R_{4b}, R_{4c}, b and a are as defined hereinbefore for a compound of formula (I), and X₁ and X₂ each represent CH or N provided that when X₁ is N, X₂ is CH and vica versa. For example, a compound of formula (III) is dissolved in a suitable solvent, such as N,N-dimethylformamide, then treated with a suitable base in the presence of an alkyl halide and heated at a suitable temperature for an appropriate time to give, after purification, compounds of the formula (IIa) wherein R₂, R₃, R₄ₐ, R_{4b}, R_{4c}, b and a are as defined hereinbefore for a compound of formula (I).

There is further provided a process for the preparation of a compound of formula (IIb):

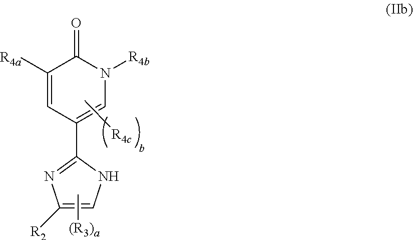

which process comprises the alkylation of a compound of formula (III):

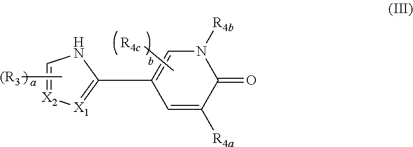

wherein R₃, R₄ₐ, R_{4b}, R_{4c}, b and a are as defined hereinbefore for a compound of formula (I), and X₁ is N and X₂ is CH. For example, a compound of formula (III) is dissolved in a suitable solvent, such as dimethylsulfoxide, then treated with suitable reagents, such as Ir(ppy)₂(dtbbpy)PF₆, tosic acid and methyl thioglycolate in the presence of an alcohol and irradiated with blue light at a suitable temperature for an appropriate time to give, after purification, compounds of formula (IIb) wherein R₂, R₃, R₄ₐ, R_{4b}, R_{4c}, b and a are as defined hereinbefore for a compound of formula (I).

There is further provided a process for the preparation of a compound of formula (IIc):

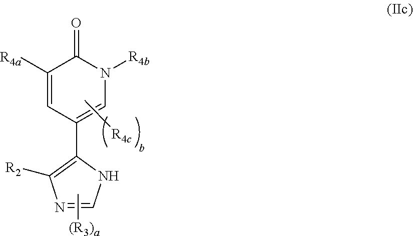

which process comprises the alkylation of a compound of formula (III):

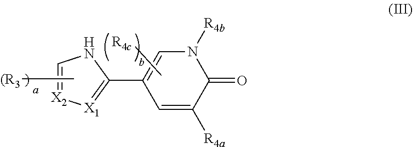

wherein R₃, R₄ₐ, R_{4b}, R_{4c}, b and a are as defined hereinbefore for a compound of formula (I), and X₁ is CH and X₂ is N. For example, a compound of formula (III) is dissolved in a suitable solvent, such as dimethylsulfoxide, then treated with suitable reagents, such as Ir(ppy)₂(dtbbpy)PF₆, tosic acid and methyl thioglycolate in the presence of an alcohol and irradiated with blue light at a suitable temperature for an appropriate time to give, after purification, compounds of the formula (IIc) wherein R₂, R₃, R₄ₐ, R_{4b}, R_{4c}, b and a are as defined hereinbefore for a compound of formula (I).

There is further provided a process for the preparation of a compound of formula (IId):

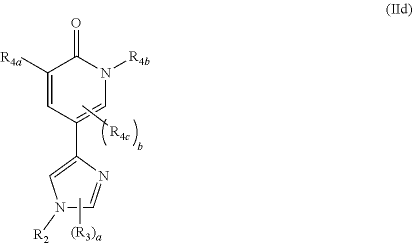

which process comprises the alkylation of a compound of formula (III):

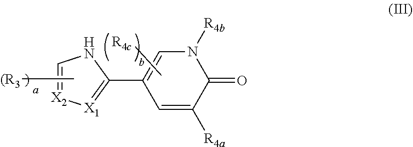

wherein R₃, R₄ₐ, R_{4b}, R_{4c}, b and a are as defined hereinbefore for a compound of formula (I), and X₁ is CH and X₂ is N. For example, a compound of formula (III) is dissolved in a suitable solvent, such as N,N-dimethylformamide, then treated with a suitable base in the presence of an alkyl halide and heated at a suitable temperature for an appropriate time to give, after purification, compounds of the formula (IId) wherein R₂, R₃, R₄ₐ, R_{4b}, R_{4c}, b and a are as defined hereinbefore for a compound of formula (I).

There is provided a process for the preparation of a compound of formula (III), which process comprises cross-coupling of a compound of formula (IV):

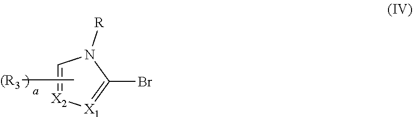

Wherein R₃ and a are as defined hereinbefore for a compound of formula (I) and R is optionally a hydrogen or suitable protecting group, such as [2-(trimethylsilyl)ethoxy]methyl acetal. X₁ and X₂ are as hereinbefore defined for a compound of formula (II). For example, a compound of formula (IV) could be dissolved in a solvent mixture such as 1,4-dioxane/water, then treated with a suitable coupling partner of formula (V) in the presence of a palladium catalyst and a suitable base, such as potassium carbonate, with heating at a suitable temperature for an appropriate time to give, after purification, compounds of the formula (III), post suitable deprotection as appropriate. The coupling partners mentioned above are of general formula (V) wherein R₄ₐ, R_{4b}, R_{4c}, and b are as defined for a compound of formula (I).

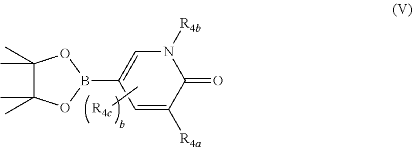

There is provided a process for the preparation of a compound of formula (II), which process comprises cross-coupling of a compound of formula (IV):

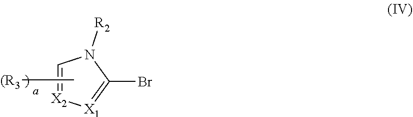

Wherein R₂, R₃ and a are as defined hereinbefore for a compound of formula (I). X₁ and X₂ are as hereinbefore defined as for a compound of formula (II). For example, a compound of formula (IV) could be dissolved in a solvent mixture such as 1,4-dioxane/water, then treated with a suitable coupling partner of formula (V) in the presence of a palladium catalyst and a suitable base, such as potassium carbonate, with heating at a suitable temperature for an appropriate time to give, after purification, compounds of the formula (II). The coupling partners mentioned above are of general formula (V) wherein R₄ is defined for a compound of formula (I).

There is further provided a process for the preparation of a compound of formula (VI):

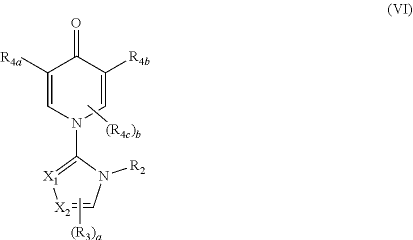

which process comprises the cross-coupling of a compound of formula (IV) above. A compound of formula (IV) could, for example, be dissolved in a suitable solvent, such as dimethyl sulfoxide, and then treated with a suitable coupling partner of formula (VII) in the presence of a copper catalyst with heating at a suitable temperature for an appropriate time to give, after purification, a compound of the formula (VI).

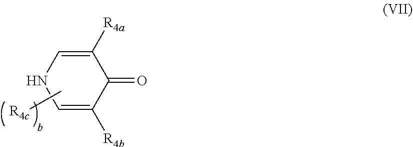

There is provided a process for the preparation of a compound of formula (IV), which process comprises the bromination of a compound of formula (VIII):

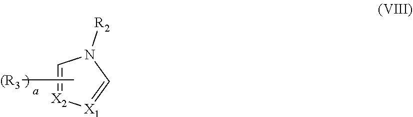

Wherein R₂, R₃ and a are as defined hereinbefore for a compound of formula (I). For example, a compound of formula (VIII) could be dissolved in a solvent such as THF then treated with a suitable base, such as TMPMgCl.LiCl, followed by a brominating agent, such as CBr₄. The mixture is then stirred at a suitable temperature for an appropriate time to give, after purification, compounds of the formula (IV).

There is provided a process for the preparation of a compound of formula (VIII), which process comprises the alkylation of a compound of formula (IX):

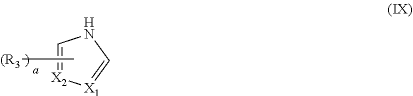

Wherein a compound of formula (IX) is dissolved in a suitable solvent, such as N,N-dimethylformamide, then treated with a suitable base, such as potassium carbonate, in the presence of an alkyl halide and heated at a suitable temperature for an appropriate time to give, after purification, compounds of the formula (VIII) wherein R₃ and a are as defined hereinbefore for a compound of formula (I).

There is provided a process for the preparation of a compound of formula (IIa), which process comprises cyclisation of a compound of the formula (X):

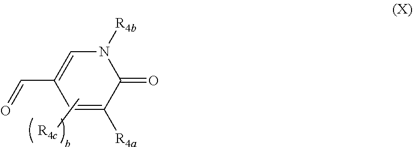

Wherein a compound of formula (X) is dissolved in a suitable solvent, such as chloroform, and then treated with a suitable amine containing R₃ as defined hereinbefore for a compound of formula (I) and a suitable 1,3-dicarbonyl compound containing R₃ as defined hereinbefore for a compound of formula (I), in the presence of a suitable acid, such as acetic acid. The mixture is then heated at a suitable temperature for an appropriate time to give, after purification, compounds of the formula (IIa).

There is provided a process for the preparation of a compound of formula (I), which process comprises functionalisation of a compound of the formula (I) wherein R₃ is a suitable functional group, such as nitrile. Such compounds may be functionalised, for example by hydrolysis and, where appropriate, further coupling to give compounds of formula (I) wherein R₂, R₃ and R₄ₐ, R_{4b}, R_{4c} are as defined hereinbefore for a compound of formula (I).

Certain compounds of formula (V), (VII), (IX) and (X) depending on the particular R₃ and R₄ substituent are commercially available from, for example, Sigma Aldrich.

### ABBREVIATIONS

CBr₄ Carbon tetrabromide
CV Column volumes

### DCM Dichloromethane

DIAD Diisopropyl azodicarboxylate

### DIPEA N,N-Diisopropylethylamine

### DMF N,N-dimethylformamide

### DMSO Dimethylsulfoxide

EtOAc Ethyl acetate

### g Grammes

h Hour(s)HPLC High-performance liquid chromatography
iPrOH isopropanol

### L Litre

LCMS Liquid chromatography-mass spectrometry

### MDAP Mass-Directed Automated Preparative HPLC

### MeCN Acetonitrile

### MeOH Methanol

MgSO₄ Magnesium sulfate

### min Minutes

### mg Milligrammes

### MHz Megahertz

### mL Millilitre

### mM Millimolar

### nm Nanometre

### NBS N-Bromosuccinimide

ppm Parts per million
RT Room temperature
TBME tert-Butyl methyl ether

### THF Tetrahydrofuran

### TMAD Tetramethylazodicarboxamide

TMPMgCl.LiCl 2,2,6,6-Tetramethylpiperidinylmagnesium chloride lithium chloride complex
TMS-Cl Trimethylsilyl chloride
t_{RET} Retention time
s seconds

### μm Micrometre

### Experimental Details

### LCMS

### System A:

The UPLC analysis was conducted on an Acquity UPLC CSH C18 column (50 mm×2.1 mm i.d. 1.7 μm packing diameter) at 40° C.

The solvents employed were:

A=0.1% v/v solution of formic acid in water.

B=0.1% v/v solution of formic acid in MeCN.

The gradient employed was:

| Time (min) | Flow (mL/min) | % A | % B |
|---|---|---|---|
| 0 | 1 | 97 | 3 |
| 1.5 | 1 | 5 | 95 |
| 1.9 | 1 | 5 | 95 |
| 2.0 | 1 | 97 | 3 |

The UV detection was a summed signal from wavelength of 210 nm to 350 nm.

Injection volume: 0.5 μL

MS Conditions

MS: Waters ZQ

Ionisation mode: Alternate-scan Positive and Negative Electrospray

Scan Range: 100 to 1000 AMU

Scan Time: 0.27 s

Inter scan Delay: 0.10 s

### System B:

The UPLC analysis was conducted on an Acquity UPLC CSH C18 column (50 mm×2.1 mm i.d. 1.7 μm packing diameter) at 40° C.

The solvents employed were:

A=10 mM ammonium bicarbonate in water adjusted to pH 10 with ammonia solution.

B=MeCN.

The gradient employed was:

| Time (min) | Flow (mL/min) | % A | % B |
|---|---|---|---|
| 0 | 1 | 97 | 3 |
| 0.05 | 1 | 97 | 3 |
| 1.50 | 1 | 5 | 95 |
| 1.90 | 1 | 5 | 95 |
| 2.00 | 1 | 97 | 3 |

The UV detection was a summed signal from wavelength of 210 nm to 350 nm.

Injection volume: 0.3 μL

MS Conditions

MS: Waters ZQ

Ionisation mode: Alternate-scan Positive and Negative Electrospray

Scan Range: 100 to 1000 AMU

Scan Time: 0.27 s

Inter scan Delay: 0.10 s

### System C:

The UPLC analysis was conducted on an Acquity UPLC CSH C18 column (50 mm×2.1 mm i.d. 1.7 μm packing diameter) at 40° C.

The solvents employed were:

A=0.1% v/v trifluoroacetic acid in water.

B=0.1% v/v trifluoroacetic acid in MeCN.

The gradient employed was:

| Time (min) | Flow (mL/min) | % A | % B |
|---|---|---|---|
| 0 | 1 | 95 | 5 |
| 1.50 | 1 | 5 | 95 |
| 1.90 | 1 | 5 | 95 |
| 2.00 | 1 | 95 | 5 |

The UV detection was a summed signal from wavelength of 210 nm to 350 nm.

Injection volume: 0.5 μL

MS Conditions

MS: Waters ZQ

Ionisation mode: Positive Electrospray

Scan Range: 100 to 1000 AMU

Scan Time: 0.27 s

Inter scan Delay: 0.05 s

System D:

The UPLC analysis was conducted on an Xbridge C18 column (50 mm×4.6 mm i.d. 2.5 μm packing diameter) at 35° C.

The solvents employed were:

A=5 mM Ammonium Bicarbonate in water (pH 10).

B=Acetonitrile

The gradient employed was:

| Time (min) | Flow (mL/min) | % A | % B |
|---|---|---|---|
| 0 | 1.3 | 95 | 5 |
| 0.5 | 1.3 | 95 | 5 |
| 1.0 | 1.3 | 85 | 15 |
| 3.3 | 1.3 | 2 | 98 |
| 5.2 | 1.3 | 2 | 98 |
| 5.5 | 1.3 | 95 | 5 |
| 6.0 | 1.3 | 95 | 5 |

The UV detection was a summed signal from wavelength of 200 nm to 400 nm.

Injection volume: 3.0 μL

MS Conditions

MS: Waters Quattro micro

Ionisation mode: Alternative-scan Positive and Negative Electrospray

Scan Range: 100 to 1000 AMU

Scan Time: 0.50 s

Inter scan Delay: 0.10 s

### Mass Directed Autopreparative HPLC (MDAP)

Mass directed autopreparative HPLC was undertaken under the conditions given below. The UV detection was an averaged signal from wavelength of 210 nm to 350 nm and mass spectra were recorded on a mass spectrometer using alternate-scan positive and negative mode electrospray ionization.

### Method A

Method A was conducted on an Xselect CSH C18 column (typically 150 mm×30 mm i.d. 5 μm packing diameter) at ambient temperature. The solvents employed were:

A=0.1% v/v solution of formic acid in water.

B=0.1% v/v solution of formic acid in acetonitrile.

### Method B

Method B was conducted on an Xselect CSH C18 column (typically 150 mm×30 mm i.d. 5 μm packing diameter) at ambient temperature. The solvents employed were:

A=10 mM Ammonium bicarbonate in water adjusted to pH 10 with Ammonia

B=Acetonitrile.

### Method C

Method C was conducted on an Xselect CSH column (typically 150 mm×30 mm i.d. 5 μm packing diameter) at ambient temperature. The solvents employed were:

A=0.1% v/v solution of TFA in water

B=0.1% v/v solution of TFA in acetonitrile.

### ¹H NMR

The ¹H NMR spectra were recorded in CDCl₃, CD₃OD or DMSO-d₆ on a Bruker AVII+400 MHz spectrometer with cryo-probe, and referenced to TMS at 0.00 ppm.

### Intermediate Preparation

Unless otherwise stated, starting materials for the preparation of Intermediates and Examples are commercially available from, for example, PharmaTech and Sigma Aldrich.

### Intermediate 1: 2,4-dibromo-1-ethyl-1H-imidazole

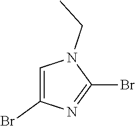

Under an atmosphere of nitrogen, sodium hydride (0.575 g, 14.39 mmol) was added to a flask containing anhydrous DMF (5 mL) cooled down using an ice bath. After a few minutes, 2,4-dibromo-1H-imidazole (2.5 g, 11.07 mmol) was added portionwise (DMF (5 mL) was added half way through addition as reagent showed poor solubility) followed by a slow addition of bromoethane (1 mL, 13.40 mmol). The resulting mixture was stirred under nitrogen, cooled down with an ice bath, for 30 min, then allowed to reach RT and left stirring for 17 h. The mixture was quenched with addition of ice-water mixture and extracted with EtOAc (×3), organics were combined and washed with brine (×3), dried on Na₂SO₄ and volatiles were removed under reduced pressure to afford 3.01 g of a runny oil. The crude was purified on a 340 g Si cartridge, eluted with a 0-20% Et₂O in cyclohexane over 20 CV. 2,5-Dibromo-1-ethyl-1H-imidazole eluted first followed by the title compound. In each case, relevant fractions were combined and volatiles removed under reduced pressure to afford the title compound (1.75 g, 6.89 mmol, 62.3%) as a white sticky solid. LCMS (System B): t_{RET}=0.82 min; MH⁺ 253, 255, 257.

### Intermediate 2: 1-(cyclopropylmethyl)-2-iodo-1H-imidazole

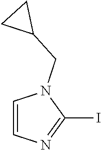

A mixture of 2-iodo-1H-imidazole (1.0 g, 5.16 mmol), (bromomethyl)cyclopropane (766 mg, 551 μL, 5.67 mmol) and potassium carbonate (2.14 g, 15.47 mmol) in acetone (20 mL) was heated under reflux for 24 h. The cooled reaction mixture was filtered and the solvent evaporated from the filtrate to give the title compound (1.12 g, 4.51 mmol, 88%), as a yellow oil. This was used without further purification. LCMS (System A): t_{RET}=0.39 min; MH⁺ 249.

### Intermediate 3: 4-chloro-1-((2-(trimethylsilyl)ethoxy)methyl)-1H-imidazole

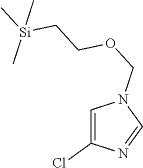

4-Chloro-1H-imidazole (2 g, 19.51 mmol) and potassium carbonate (5.39 g, 39.0 mmol) were added to a round bottomed flask containing a stirrer bar and placed under an atmosphere of nitrogen by evacuation-refill. DMF (25 mL) was added, evacuation-refill of the vessel repeated, and the mixture stirred prior to addition of (2-(chloromethoxy)ethyl)trimethylsilane (6.91 mL, 39.0 mmol) in DMF (25 mL). The reaction vessel was placed under an atmosphere of nitrogen and left to stir at RT. After 3.5 h, the reaction mixture was taken forwards for work up. The reaction mixture was quenched with 20 mL water, and the solvent was removed under reduced pressure. The residue was dissolved in 50 mL EtOAc, and washed with 30 mL water, then 30 mL brine. The organic layer was passed through a hydrophobic frit and the solvent removed under reduced pressure. The sample was loaded in a minimum of dichloromethane and purified by gradient elution column chromatography using a 120 g silica cartridge eluting with a 0-30% ethyl acetate-cyclohexane solvent system. The appropriate fractions were combined and evaporated in vacuo to give the title compound as a pale yellow oil (2.37 g). LCMS: (System A): t_{RET}=1.16 min; MH⁺ 233, 235.

Intermediate 4: 2-bromo-4-chloro-1-((2-(trimethlsilyl)ethoxy)methyl)-1H-imidazole

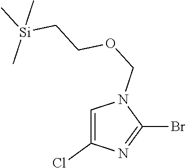

To a stirred solution of 4-chloro-1-((2-(trimethylsilyl)ethoxy)methyl)-1H-imidazole (for an example preparation see Intermediate 3, 2.00 g, 8.59 mmol) in THF (22 mL) under an atmosphere of nitrogen at 0° C. was added 1M TMPMgCl.LiCl (12.89 mL, 12.89 mmol) dropwise. The reaction was stirred for 1 h at this temperature, and then CBr₄ (5.70 g, 17.18 mmol) in THF (20 mL) were added dropwise over 5 min. The reaction was allowed to slowly warm to RT and stirred for a further 3 h. The reaction was quenched by the addition of NaHCO₃ saturated aq. solution (5 mL) and extracted with DCM (3×5 mL). The combined organic layers were dried through a hydrophobic filter and the solvent removed in vacuo. The crude sample was dissolved in DCM (10 mL) and loaded directly onto a 120 g silica column (prewashed with hexane). Purification by flash column chromatography eluting with cyclohexane to 30% EtOAc in cyclohexane over 30 CV afforded the title compound (1.86 g, 5.67 mmol, 66%) as a light brown oil. LCMS: (System A): t_{RET}=1.30 min; MH⁺ 311, 313, 315.

### Intermediate 5: 5-(4-chloro-1-((2-(trimethylsilyl)ethoxy)methyl)-1H-imidazol-2-yl)-1,3-dimethylpyridin-2(1H)-one

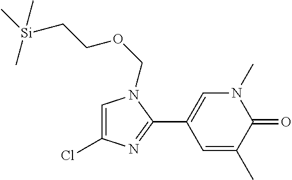 [SMILES: Cc1cc(-c2nc(Cl)cn2COCC[Si](C)(C)C)cn(C)c1=O]

To two 20 mL microwave vials was added potassium carbonate (1 g, 7.24 mmol), 1,3-dimethyl-5-(4,4,5,5-tetramethyl-1,3,2-dioxaborolan-2-yl)pyridin-2(1H)-one (commercially available from, for example, Milestone PharmaTech, 0.863 g, 3.47 mmol) and 2-bromo-4-chloro-1-((2-(trimethylsilyl)ethoxy)methyl)-1H-imidazole (for an example preparation, see Intermediate 4, 0.9 g, 2.89 mmol), 1,4-dioxane (10 mL) and water (2.5 mL) and purged with nitrogen for 5 min. Tetrakis(triphenylphosphine)palladium(0) (0.100 g, 0.087 mmol) was added, the vial sealed, and purged with nitrogen for a further 5 min. The reaction was stirred at 110° C. in a microwave reactor for 1 h. The two vials were combined and the solvent was removed in vacuo, the crude residue taken up in ethyl acetate (20 mL) and filtered through celite (washing with 3×20 mL) EtOAc. The solvent was removed in vacuo. The crude residue was dissolved in DCM (10 mL) and loaded onto a 120 g silica column (prewashed with cyclohexane). Purification by flash column chromatography eluting with 100% cyclohexane to 100% EtOAc over 30 CV afforded the title compound (921 mg, 2.60 mmol, 45%) as a light yellow solid. LCMS: (System A): t_{RET}=1.16 min; MH⁺ 354, 356.

### Intermediate 6: methyl 1-((2-(trimethylsilyl)ethoxy)methyl)-1H-imidazole-4-carboxylate

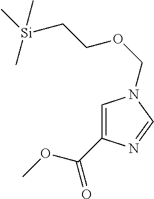 [SMILES: COC(=O)c1cn(COCC[Si](C)(C)C)cn1]

Methyl 1H-imidazole-4-carboxylate (2 g, 15.86 mmol) and potassium carbonate (4.38 g, 31.7 mmol) were added to a round bottomed flask containing a stirrer bar and placed under an atmosphere of nitrogen by evacuation-refill. Acetone (20 mL) was added, evacuation-refill of the vessel repeated, and the mixture stirred prior to addition of (2-(chloromethoxy)ethyl)trimethylsilane (3.37 mL, 19.03 mmol). The reaction vessel was placed under an atmosphere of nitrogen and left stirring overnight at RT. A further 0.33 equivalents of (2-(chloromethoxy)ethyl)trimethylsilane (0.926 mL, 5.23 mmol) were added and the reaction left to continue for a further 4 h. The reaction mixture was quenched with addition of 40 mL water and extracted with EtOAc (40 mL), with the addition of 10 mL brine to prevent formation of a triphasic solution. The aqueous layer was extracted with a further 3×40 mL EtOAc. The organic layers were combined, passed through a hydrophobic frit, and the solvent removed under reduced pressure. The sample was dissolved in DCM and purified by flash chromatography using a silica 120 g cartridge, using a solvent system of 10-75% ethyl acetate-cyclohexane over 25 CV. The appropriate fractions were combined and evaporated in vacuo to give the following two products:

methyl 1-((2-(trimethylsilyl)ethoxy)methyl)-1H-imidazole-5-carboxylate (1.45 g). LCMS (System B): t_{RET}=1.10 min; MH⁺ 257.

methyl 1-((2-(trimethylsilyl)ethoxy)methyl)-1H-imidazole-4-carboxylate (the title compound) (1.34 g). LCMS (System B): t_{RET}=1.02 min; MH⁺ 257.

### Intermediate 7: methyl 2-bromo-1-((2-(trimethylsilyl)ethoxy)methyl)-1H-imidazole-4-carboxylate

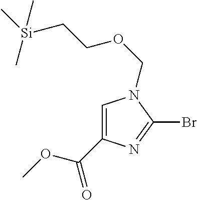 [SMILES: COC(=O)c1cn(COCC[Si](C)(C)C)c(Br)n1]

Methyl 1-((2-(trimethylsilyl)ethoxy)methyl)-1H-imidazole-4-carboxylate (for an example preparation, see Intermediate 6, 297 mg, 1.158 mmol) was added to a round bottomed flask containing trifluorotoluene (6 mL). Once dissolved, azobisisobutyronitrile (9.51 mg, 0.058 mmol) and N-bromosuccinimide (227 mg, 1.274 mmol) were added, and the flask placed under an atmosphere of nitrogen. The reaction mixture was stirred at 65° C. overnight. The reaction mixture was quenched with saturated sodium hydrogencarbonate solution (20 mL) and extracted with EtOAc (2×20 mL). The organic layers were combined and the solvent removed under reduced pressure. The sample was loaded in DCM and purified by column chromatography using a silica cartridge (80 g) with an ethyl acetate-cyclohexane solvent system [10-20%, 1CV; 20%, 7CV; 20-100%, 3CV; 100%, 3CV]. The appropriate fractions were combined and the solvent removed in vacuo to afford the title compound as a white solid (206 mg, 0.61 mmol, 53%). LCMS (System B): t_{RET}=1.16 min; MH⁺ 335, 337.

### Intermediate 8: methyl 2-(1,5-dimethyl-6-oxo-1,6-dihydropyridin-3-yl)-1-((2-(trimethylsilyl)ethoxy)methyl)-1H-imidazole-4-carboxylate

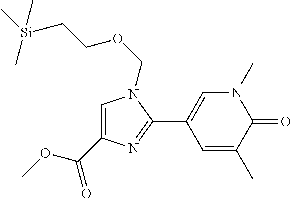 [SMILES: COC(=O)c1cn(COCC[Si](C)(C)C)c(-c2cc(C)c(=O)n(C)c2)n1]

1,3-Dimethyl-5-(4,4,5,5-tetramethyl-1,3,2-dioxaborolan-2-yl)pyridin-2(1H)-one (commercially available from, for example, Milestone PharmaTech, 1.739 g, 6.98 mmol), methyl 2-bromo-1-((2-(trimethylsilyl)ethoxy)methyl)-1H-imidazole-4-carboxylate (for an example preparation, see Intermediate 7, 1.56 g, 4.65 mmol), and potassium carbonate (1.929 g, 13.96 mmol) were added to a 5 mL microwave vial containing a stirrer bar. 1,4-Dioxane (15 mL) and methanol (5 mL) were added to the vial, which was purged with nitrogen for 5 mins prior to the addition of tetrakis(triphenylphosphine)palladium(0) (0.161 g, 0.140 mmol). After a further 5 min purge with nitrogen, the vial was capped and heated in the microwave at 100° C. for 1 h. A further 0.5 equivalents of 1,3-dimethyl-5-(4,4,5,5-tetramethyl-1,3,2-dioxaborolan-2-yl)pyridin-2(1H)-one (for an example preparation, see Intermediate 3, 0.580 g, 2.326 mmol) and 1 mol % of tetrakis(triphenylphosphine)palladium(0) (0.054 g, 0.047 mmol) were added to the microwave vial, which was purged with nitrogen for a further 10 min and returned to the microwave for another 1 h of heating at 100° C. The solvent from the reaction mixture was removed by evaporation under reduced pressure. The residue was redissolved in ethyl acetate and filtered through Celite® to remove any aqueous soluble impurities, the solvent was removed under reduced pressure. The sample was loaded in DCM and purified by column chromatography using a silica column (120 g) with an ethyl acetate-cyclohexane solvent system [25-75%, 15CV; 75%, 10CV]. The appropriate fractions were combined and evaporated in vacuo to give the crude product. The crude product was redissolved in ethyl acetate (30 mL) and washed with 8 portions of water/brine (30 mL/10 mL) until all traces of impurity had been removed from the organic layer. The organic layer was passed through a hydrophobic frit and the solvent removed under reduced pressure to yield the title compound as a beige solid (1.76 g). LCMS (System B): t_{RET}=1.06 min; MH⁺ 378.

### Intermediate 9: 1-((2-(trimethylsilyl)ethoxy)methyl)-1H-imidazole-4-carbonitrile

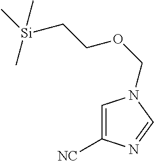

1H-Imidazole-4-carbonitrile (1 g, 10.74 mmol) and potassium carbonate (2.97 g, 21.49 mmol) were added to a round bottomed flask containing a stirrer bar and placed under an atmosphere of nitrogen by evacuation-refill. Acetone (10 mL) was added, evacuation-refill of the vessel repeated, and the mixture stirred prior to addition of (2-(chloromethoxy)ethyl)trimethylsilane (2.28 mL, 12.89 mmol). The reaction vessel was placed under an atmosphere of nitrogen and left for 48 h with stirring at RT. The solvent was removed under reduced pressure, and the residue dissolved in 30 mL EtOAc, then washed with 30 mL water and 20 mL brine. The combined aqueous layers were extracted with EtOAc (2×30 mL). The organic layers were combined and passed through a hydrophobic frit, the solvent removed under reduced pressure. The sample was dissolved in DCM and purified with gradient elution flash chromatography using a 80 g silica cartridge, using a solvent system of 10-75% ethyl acetate-cyclohexane over 20 CV. The appropriate fractions were combined and evaporated in vacuo to give the title compound as a clear oil (1.61 g) which contained about 10% of the 5-carbonitrile regioisomer in addition to the major 4-carbonitrile product. LCMS (System B): t_{RET}=1.08 min; MH⁺ 224.

### Intermediate 10: 2-bromo-1-((2-(trimethylsilyl)ethoxy)methyl)-1H-imidazole-4-carbonitrile

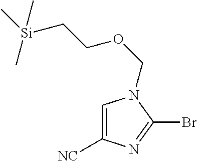 [SMILES: C[Si](C)(C)CCOCn1cc(C#N)nc1Br]

1-((2-(Trimethylsilyl)ethoxy)methyl)-1H-imidazole-4-carbonitrile (for an example preparation, see Intermediate 9, 1.41 g, 6.31 mmol) was added to a round bottomed flask containing THF (30 mL) and a stirrer bar. Once dissolved, NBS (1.236 g, 6.94 mmol) was added, the flask was placed under an atmosphere of nitrogen. The reaction mixture was heated to 60° C. and left overnight, with stirring. A further 0.25 equivalents of NBS (0.281 g, 1.578 mmol) was added to the reaction mixture and the reaction left stirring at 60° C. for a further 5 h. The solvent was removed under reduced pressure and the residue redissolved in EtOAc (30 mL). The reaction mixture was washed with water (30 mL) and brine (20 mL) and the aqueous layer extracted with EtOAc (2×30 mL). The combined organic layers were passed through a hydrophobic frit and the solvent removed under reduced pressure. The sample was absorbed onto Florisil® from a methanol solution, and purified by gradient elution column chromatography using a 80 g silica cartridge using a 0-50% ethyl acetate-cyclohexane solvent system. The appropriate fractions were combined and evaporated in vacuo to give the title compound as a cloudy oil (943 mg). LCMS (System B): t_{RET}=1.25 min; MH⁺ not detected.

### Intermediate 11: 2-(1,5-dimethyl-6-oxo-1,6-dihydropyridin-3-yl)-1-((2-(trimethylsilyl)ethoxy)methyl)-1H-imidazole-4-carbonitrile

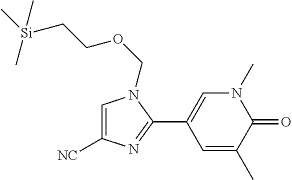 [SMILES: Cc1cc(-c2nc(C#N)cn2COCC[Si](C)(C)C)cn(C)c1=O]

1,3-Dimethyl-5-(4,4,5,5-tetramethyl-1,3,2-dioxaborolan-2-yl)pyridin-2(1H)-one (commercially available from, for example, Milestone PharmaTech, 1166 mg, 4.68 mmol), 2-bromo-1-((2-(trimethylsilyl)ethoxy)methyl)-1H-imidazole-4-carbonitrile (for an example preparation, see Intermediate 10, 943 mg, 3.12 mmol), and potassium carbonate (1294 mg, 9.36 mmol) were added to a 5 mL microwave vial containing a stirrer bar. 1,4-Dioxane (15 mL) and water (5 mL) were added to the vial, which was purged with nitrogen for 5 min prior to the addition of tetrakis(triphenylphosphine)palladium(0) (108 mg, 0.094 mmol). After a further 5 min purge with nitrogen, the vial was capped and heated in the microwave at 110° C. for 1 h. The solvent was removed by evaporation under reduced pressure. The residue was redissolved in ethyl acetate and filtered through Celite®, the solvent again removed under reduced pressure. The sample was loaded in DCM and purified using gradient elution column chromatography with a 80 g silica cartridge using an 5-75% ethyl acetate-cyclohexane solvent system over 20 CVs. The appropriate fractions were combined and evaporated in vacuo to give the title compound as a white solid (551 mg). A second, less pure, batch was also isolated. This sample was dissolved in EtOAc (30 mL) and subjected to repeated washes with water (8×50 mL) until the impurity was no longer visible in the organic layer. The purified second batch was obtained as a white solid (297 mg). LCMS (System B): t_{RET}=1.11 min; MH⁺ 354.

### Intermediate 12: 4-chloro-1-(1,3-dimethoxypropan-2-yl)-1H-imidazole

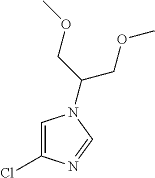

Tri-n-butylphosphine (2.407 mL, 9.75 mmol) and 4-chloro-1H-imidazole (100 mg, 0.975 mmol) were dissolved in toluene (10 mL) at 0° C. 1,3-Dimethoxypropan-2-ol (1.161 mL, 9.75 mmol) was added followed by TMAD (840 mg, 4.88 mmol) and the reaction stirred at this temperature for 10 min. The reaction was then heated to 60° C. for 8 h, then at 80° C. for a further 16 h. The solvent was removed in vacuo, and the crude residue purified by MDAP (Method B) to afford the product as a colourless oil (126 mg, 0.585 mmol, 60%) as 3:1 mixture of chloro regioisomers. A sample (100 mg) was dissolved in 1:1 MeOH:DMSO (3 mL) and purified by MDAP (Method C). The solvent was evaporated in vacuo to yield the first (undesired) regioisomer as a clear oil (15 mg). The solvent waste was evaporated under reduced pressure and the residue extracted into EtOAc (100 mL) prior to washing with sat NaHCO₃ solution and brine (100 mL ea.). The solvent was removed from the organic layer under reduced pressure. This sample was dissolved in 1:1 MeOH:DMSO (3 mL) and purified by MDAP (Method C). The solvent was concentrated under reduced pressure and neutralised with addition of sat NaHCO₃ solution. The second product was extracted into EtOAc (2×100 mL) and the solvent removed under reduced pressure to yield the second regioisomer (the title compound) as a clear oil (65 mg). LCMS (System B): t_{RET}=0.71 min; MH⁺ 205, 207.

### Intermediate 13: 2-bromo-4-chloro-1-(1,3-dimethoxypropan-2-yl)-1H-imidazole

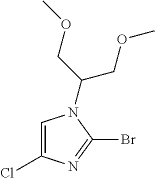

A solution of 4-chloro-1-(1,3-dimethoxypropan-2-yl)-1H-imidazole (for an example preparation, see Intermediate 12, 64 mg, 0.313 mmol) in THF (1.5 mL) was prepared in a dried 2-5 mL microwave vial and cooled to 0° C. under an atmosphere of nitrogen. TMPMgCl.LiCl (1M in THF/toluene) (0.469 mL, 0.469 mmol) was added dropwise and the reaction stirred for 1 h. A solution of CBr₄ (207 mg, 0.625 mmol) in THF (1 mL) was prepared in a second dried microwave vial under nitrogen, and this solution was transferred dropwise by syringe to the first reaction vessel. The reaction mixture was allowed to reach RT and stirred for a further 4 h under nitrogen. The solvent was removed under reduced pressure and the residue redissolved in EtOAc (50 mL). This was washed with sat NaHCO₃ solution (50 mL), before passing the organic layer through a hydrophobic frit and removing the solvent under reduced pressure. The sample was dissolved in 1:1 MeOH:DMSO (3 mL) and purified by MDAP (Method B). The solvent was evaporated in vacuo to give title compound as a yellow oil (55 mg). LCMS (System B): t_{RET}=0.89 min; MH⁺ 283, 285, 287.

### Intermediate 14: rac-tert-butyl 3-((4-chloro-1H-imidazol-1-yl)methyl)piperidine-1-carboxylate

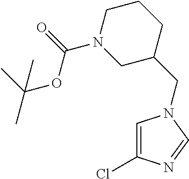 [SMILES: CC(C)(C)OC(=O)N1CCCC(Cn2cnc(Cl)c2)C1]

4-chloro-1H-imidazole (2 g, 19.51 mmol), DIPEA (6.81 mL, 39.0 mmol) and K₂CO₃ (5.39 g, 39.0 mmol) were combined in DMF (100 mL) under nitrogen and stirred for 5 mins. tert-Butyl 3-(bromomethyl)piperidine-1-carboxylate (7.60 g, 27.3 mmol) was added and the reaction heated to 100° C. overnight. The reaction was cooled and filtered then concentrated in vacuo to give a yellow semi-solid. The residue was taken up in MeOH (20 mL) and 8 mL was applied to a 60 g C-18 silica which was eluted with 0% (MeCN+0.1% Formic acid) in (water+0.1% Formic acid) for 2 CV then 0-50% (MeCN+0.1% Formic acid) over 10 CV then held at 50% for 5 CV. The appropriate fractions were combined and concentrated in vacuo to give the title compound (Batch 1) as a clear oil. The remaining crude product was purified using the same gradient and a 120 g silica column (crude solution was slightly cloudy so a couple of drops of water were added to solubilise). The appropriate fractions were concentrated in vacuo to give a clear oil. This oil was purified further using a 120 g silica column and the elution conditions described above. The appropriate fractions were concentrated in vacuo to give the title compound (Batch 2) as a clear oil. Mixed fractions (from the above described purifications) were combined and concentrated in vacuo to give a yellow oil. This was taken up in the minimum of MeOH and divided into two portions and each purified on a 120 g silica column using the same gradient as above. The appropriate fractions from the columns were combined and concentrated in vacuo to give the title compound (Batch 3) as a yellow oil. The three batches of title compound were combined in the minimum of MeOH and then concentrated in vacuo to give a single batch of the title compound (3.57 g) as a yellow oil. LCMS (System B) t_{RET}, 1.05 mins, MH⁺=300, 302.

### Intermediate 15: rac-tert-butyl 3-((2-bromo-4-chloro-1H-imidazol-1-yl)methyl)piperidine-1-carboxylate

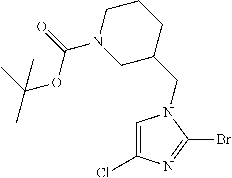 [SMILES: CC(C)(C)OC(=O)N1CCCC(Cn2cc(Cl)nc2Br)C1]

tert-butyl 3-((4-chloro-1H-imidazol-1-yl)methyl)piperidine-1-carboxylate (Intermediate 14, 3.565 g, 11.89 mmol) was taken up in THF (30 mL) under nitrogen and cooled in an ice-bath. TMPMgCl.LiCl (1M in THF) (17.84 mL, 17.84 mmol) was added dropwise over −10 mins and the reaction stirred for 30 mins. CBr₄ (7.89 g, 23.78 mmol) in THF (30 mL) was added dropwise and the reaction left to stir and warm up overnight. The reaction was cooled in an ice-bath and quenched with sat. NaHCO₃ (50 mL) then extracted with EtOAc (3×50 mL). The combined organics were washed with brine (250 mL) then eluted through a hydrophobic frit and concentrated in vacuo to give a brown oil. The oil was taken up in the minimum of DCM and divided into two. Each portion was applied to a 100 g SNAP cartridge and eluted with 0% ethyl acetate in cyclohexane for 2 CV then 0-50% ethyl acetate over 10 CV then held at 50% for 5 CV. The appropriate fractions from each column were combined and concentrated in vacuo to give the title compound (3.606 g, 76%) as a dark orange oil. LCMS (System B) t_{RET}, 1.20 mins, MH+=378, 380, 382.

### Intermediate 16 rac-tert-butyl 3-((4-chloro-2-(1,5-dimethyl-6-oxo-1,6-dihydropyridin-3-yl)-1H-imidazol-1-yl)methyl)piperidine-1-carboxylate

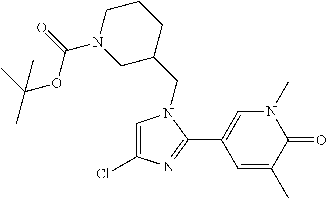 [SMILES: Cc1cc(-c2nc(Cl)cn2CC2CCCN(C(=O)OC(C)(C)C)C2)cn(C)c1=O]

tert-Butyl 3-((2-bromo-4-chloro-1H-imidazol-1-yl)methyl)piperidine-1-carboxylate (for an example preparation, see Intermediate 15, 3.6 g, 9.51 mmol) was taken up in 1,4-Dioxane (24 mL) and Water (6 mL). Nitrogen was bubbled through the solution for 10 mins and then 1,3-dimethyl-5-(4,4,5,5-tetramethyl-1,3,2-dioxaborolan-2-yl)pyridin-2(1H)-one (commercially available from, for example, Milestone PharmaTech, 4.74 g, 19.01 mmol), K₂CO₃ (3.94 g, 28.5 mmol) and tetrakis(triphenylphosphine)palladium(0) (0.549 g, 0.475 mmol) were added. The reaction was heated to 110° C. under nitrogen for 4 h. The reaction was cooled and diluted with EtOAc (50 mL) then filtered through Celite®. The filter cake was washed with EtOAc (25 mL) and the filtrate washed with water and brine (100 mL each), dried with Na₂SO₄, filtered through a hydrophobic frit and concentrated in vacuo to yield an orange oil. The crude product was applied to a 340 g SNAP cartridge in the minimum of DCM and eluted with 10% (3:1 EtOAc:EtOH) in cyclohexane for 2 CV then 10-60% (3:1 EtOAc:EtOH) over 10 CV then held at 60% for 5 CV. The appropriate fractions were concentrated in vacuo to give the title compound (3.317 g, 79%) as a cream foam. LCMS (System B) t_{RET}, 1.20 mins, MH+=421, 423.

### Intermediate 17: rac-5-(4-chloro-1-(piperidin-3-ylmethyl)-1H-imidazol-2-yl)-1,3-dimethylpyridin-2(1H)-one

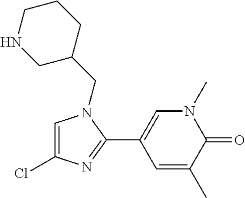 [SMILES: Cc1cc(-c2nc(Cl)cn2CC2CCCNC2)cn(C)c1=O]

tert-butyl 3-((4-chloro-2-(1,5-dimethyl-6-oxo-1,6-dihydropyridin-3-yl)-1H-imidazol-1-yl)methyl)piperidine-1-carboxylate (for an example preparation, see Intermediate 16, 2.8176 g, 6.69 mmol) was taken up in DCM (50 mL) and TFA (10 mL, 130 mmol) added. The reaction was stirred at RT for 1 h. The solvent was removed in vacuo and the residue was take up in MeOH and applied to a 20 g SCX cartridge which was eluted with MeOH then 2N NH₃ in MeOH (100 mL each). The fractions eluted with 2N NH₃ in MeOH were concentrated in vacuo to give the title compound (1.99 g, 88%) as a yellow oil. LCMS (System B) t_{RET}, 0.68 mins, MH⁺=321, 323.

### Intermediate 18: rac-4-chloro-1-((tetrahydro-2H-pyran-3-yl)methyl)-1H-imidazole

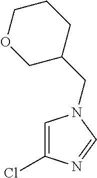 [SMILES: Clc1cn(CC2CCCOC2)cn1]

To stirred 4-chloro-1H-imidazole (6 g, 58.5 mmol) and potassium carbonate (16.18 g, 117 mmol) was added a solution of 3-(bromomethyl)tetrahydro-2H-pyran (15.72 g, 88 mmol) in anhydrous DMF (200 mL), the resulting mixture was stirred at 100° C. under a nitrogen atmosphere for 16 h. The reaction mixture was concentrated in vacuo and the residue partitioned between water (800 mL) and ethyl acetate (800 mL). The organic phase was separated and the aqueous phase was back extracted with ethyl acetate (250 mL). The combined organic extracts were dried (MgSO₄), filtered and concentrated in vacuo to give the crude product (12.19 g). The crude product was dissolved in ethyl acetate and purified on a silica cartridge (330 g) using a 0-10% ethanol-ethyl acetate (+1% Et₃N) gradient over 12 CV. The appropriate fractions were combined and evaporated in vacuo to furnish the title compound (7.99 g, 68%). LCMS (System B): t_{RET}=0.70 min; MH⁺ 201, 203.

In addition, following concentration in vacuo of the appropriate fractions furnished 5-chloro-1-((tetrahydro-2H-pyran-3-yl)methyl)-1H-imidazole (1.85 g, 16%). LCMS (System B): t_{RET}=0.74 min; MH⁺ 201, 203.

### Intermediate 19: rac-2-bromo-4-chloro-1-((tetrahydro-2H-pyran-3-yl)methyl)-1H-imidazole

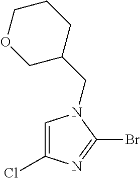 [SMILES: Clc1cn(CC2CCCOC2)c(Br)n1]

To a stirred solution of 4-chloro-1-((tetrahydro-2H-pyran-3-yl)methyl)-1H-imidazole (for an example preparation, see Intermediate 18, 7.98 g, 39.8 mmol) in anhydrous THF (80 mL) at 0° C. under a nitrogen atmosphere was added TMPMgCl.LiCl 1.0 M in THF/toluene (80 mL, 80 mmol) dropwise over 30 min. The resulting brown mixture was stirred at 0° C. for 1 h. To the reaction mixture was added a solution of CBr₄ (39.6 g, 119 mmol) in anhydrous THF (55 mL) dropwise over 20 min. The reaction mixture was then allowed to warm to RT (removal of ice bath), and stirred for a further 16 h. The reaction was quenched by the careful addition of water (20 mL) with cooling (reaction flask placed in a cold water bath). The particulate matter (formed upon quenching) was removed by filtration and the filter cake washed with ethyl acetate (100 mL). The combined filtrates were concentrated in vacuo to give a viscous brown oil (37 g). The residue was partitioned between ethyl acetate (500 mL) and saturated aqueous sodium bicarbonate (500 mL). The organic phase was separated and the aqueous phase was back extracted with ethyl acetate (250 mL). The combined organic extracts were then extracted with 2N aqueous hydrochloric acid (2×500 mL). The organic phase was washed with brine (250 mL), dried (MgSO₄), filtered and concentrated in vacuo to give a brown oil (21.7 g). The aqueous phase was adjusted to pH>14 using solid sodium hydroxide and extracted with ethyl acetate (2×500 mL). The combined organic extracts were washed with brine (250 mL), dried (MgSO₄), filtered and concentrated in vacuo to give (4.06 g) of a brown oil. The isolated brown oils were combined and were dissolved in DCM and purified on a silica cartridge (330 g) using a 0-50% ethyl acetate+1% Et₃N-cyclohexane gradient over 12 CV. The appropriate fractions were combined and evaporated in vacuo to furnish the title compound (9.113 g, 82%) as a brown oil. LCMS (System B): t_{RET} 0.88 min; MH⁺=279, 281, 283.

### Intermediate 20: 4-chloro-1-((tetrahydro-2H-pyran-4-yl)methyl)-1H-imidazole

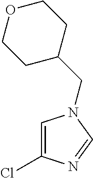 [SMILES: Clc1cn(CC2CCOCC2)cn1]

To stirred 4-chloro-1H-imidazole (27.2 g, 265 mmol) and potassium carbonate (73.3 g, 531 mmol) was added a solution of 4-(bromomethyl)tetrahydro-2H-pyran (66.5 g, 371 mmol) in anhydrous DMF (1000 mL), the resulting mixture was stirred at an internal temperature of 100° C. under a nitrogen atmosphere for 18 h. After cooling to RT the reaction mixture was filtered and the filter cake washed with MeCN (50 mL). The combined filtrates were concentrated in vacuo to give a brown oil with some particulate matter present. This residue was triturated with ethyl acetate (200 mL) and filtered. The filter cake was washed with ethyl acetate (50 mL). The combined filtrates were concentrated in vacuo to give a brown oil (59.65 g). The oil was dissolved in ethyl acetate (50 mL) and purified on a silica cartridge (1.5 Kg) using a 0-10% ethanol-ethyl acetate (+1% Et₃N) gradient over 12 CV. The appropriate fractions were combined and evaporated in vacuo to furnish the title compound (29.4 g, 55%) as an orange oil. LCMS (System B) t_{RET} 0.67 min, MH+=201, 203.

In addition concentration in vacuo of the relevant fractions furnished 5-chloro-1-((tetrahydro-2H-pyran-4-yl)methyl)-1H-imidazole (8.47 g, 16%) as an orange oil. LCMS (System B) t_{RET} 0.71 min; MH⁺=201, 203.

### Intermediate 21: 2-bromo-4-chloro-1-((tetrahydro-2H-pyran-4-yl)methyl)-1H-imidazole

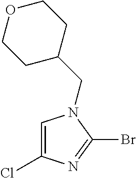 [SMILES: Clc1cn(CC2CCOCC2)c(Br)n1]

To a stirred solution of 4-chloro-1-((tetrahydro-2H-pyran-4-yl)methyl)-1H-imidazole (for an example preparation, see Intermediate 20, 28.64 g, 143 mmol) in anhydrous THF (250 mL) at 0° C. under a nitrogen atmosphere was added TMPMgCl.LiCl solution 1.0 M in THF/toluene (186 mL, 186 mmol) dropwise over 45 min, maintaining an internal temperature of 0-4° C. The resulting solution was allowed to warm to RT (removal of ice bath) and stirred at RT for 60 min. To the reaction mixture was added a solution of CBr₄ (61.5 g, 186 mmol) in anhydrous THF (250 mL) dropwise over 60 mins, maintaining an internal temperature of 17-24° C. The resulting brown solution was stirred at RT for 2.5 h. The reaction was quenched by the slow addition of water (65 mL). The resulting suspension was filtered and the filter cake washed with ethyl acetate (800 mL). The combined filtrates were concentrated in vacuo to give a semi-solid brown gum. The gum was partitioned between water (1 L) and ethyl acetate (800 mL), the organic phase was separated and the aqueous phase further extracted with ethyl acetate (400 mL). The combined organic extracts were dried (MgSO₄), filtered and concentrated in vacuo to give a viscous brown oil (76.4 g). The oil was dissolved in DCM and purified on a silica cartridge (750 g) using a 0-50% ethyl acetate+1% Et₃N-cyclohexane gradient over 12 CV. The appropriate fractions were combined and evaporated in vacuo to a brown solid (30.8 g). This solid was triturated with petroleum ether 40-60 (50 mL). The mother liquor was decanted and the resulting solid dried in vacuo to furnish the title compound (29.8 g, 75%) as a brown solid. LCMS (System B) t_{RET}, 0.84 min, MH⁺=279, 281, 283.

### Intermediate 22: 3:1 mixture of 4-bromo-1-ethyl-1H-imidazole and 5-bromo-1-ethyl-1H-imidazole

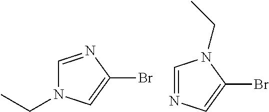

A mixture of 4-bromo-1H-imidazole (3.0 g, 20.4 mmol), potassium carbonate (8.46 g, 61.2 mmol) and iodoethane (4.78 g, 2.47 mL, 30.6 mmol) in acetone (30 mL) was refluxed for 24 h. The cooled reaction mixture was filtered and the solvent evaporated from the filtrate. The residue was chromatographed [0-10% ethanol/ethyl acetate] to give the title compound as a colourless oil (480 mg). LCMS (System B) t_{RET}=0.61 min and 0.67 min; MH⁺=175, 177 and 175, 177.

### Intermediate 23: methyl 2-(1,5-dimethyl-6-oxo-1,6-dihydropyridin-3-yl)-1H-imidazole-4-carboxylate

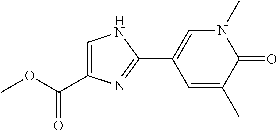 [SMILES: COC(=O)c1c[nH]c(-c2cc(C)c(=O)n(C)c2)n1]

Methyl 2-(1,5-dimethyl-6-oxo-1,6-dihydropyridin-3-yl)-1-((2-(trimethylsilyl)ethoxy)methyl)-1H-imidazole-4-carboxylate (for an example preparation, see Intermediate 8, 1.76 g, 4.66 mmol) was added to a round bottomed flask containing a stirrer bar and dissolved in anhydrous methanol (20 mL). The flask was purged with nitrogen by evacuation-refill, and trimethylsilylchloride (11.92 mL, 93 mmol) added to the reaction mixture. The reaction mixture was stirred at 40° C. for 18 h under an atmosphere of nitrogen. The solvent was removed under reduced pressure, and the crude product twice redissolved in methanol (30 mL) and the solvent removed in vacuo. The crude product was loaded in methanol and purified by SPE using a 20 g sulphonic acid (SCX) cartridge, with sequential solvent elution of methanol followed by 2M ammonia in methanol. The appropriate fractions were combined and the solvent removed in vacuo to give the title compound as a white solid, (773 mg). LCMS (System B): t_{RET}=0.56 min; MH⁺ 248.

### Intermediate 24: rac-2,4-dibromo-1-((tetrahydro-2H-pyran-3-yl)methyl)-1H-imidazole

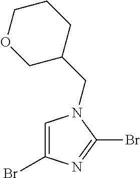 [SMILES: Brc1cn(CC2CCCOC2)c(Br)n1]

To a solution of 2,4-dibromo-1H-imidazole (300 mg, 1.328 mmol) dissolved in DMF (3.8 mL), was added 3-(bromomethyl)tetrahydro-2H-pyran (0.192 mL, 1.461 mmol) and potassium carbonate (551 mg, 3.98 mmol). The reaction mixture was purged with nitrogen and stirred at 100° C. for 45 mins under microwave irradiation. The solvent was removed under reduced pressure, and the residue dissolved in EtOAc (15 mL). The organic layer was washed with saturated sodium hydrogen carbonate solution (15 mL), brine (15 mL), and the aqueous layers extracted with EtOAc (2×15 mL). The organic layers were combined, passed through a hydrophobic frit and the solvent removed under reduced pressure to afford a yellow oil. The resulting residue was dissolved in 3 mL DCM and was purified using a 40 g normal phase silica column, eluting with cyclohexane to 30% EtOAc (+1% NEt₃) in cyclohexane to afford the title compound as a colourless oil (160 mg). LCMS (System A): t_{RET}=0.88 min; MH⁺ 323, 325, 327.

### Intermediate 25: 3:1 mixture of 2,4-dibromo-1-((tetrahydro-2H-pyran-4-yl)methyl)-1H-imidazole and 2,5-dibromo-1-((tetrahydro-2H-pyran-4-yl)methyl)-1H-imidazole

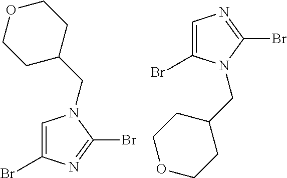

To a solution of 2,4-dibromo-1H-imidazole (200 mg, 0.885 mmol) dissolved in DMF (2.5 mL), were added potassium carbonate (367 mg, 2.66 mmol) and 4-(bromomethyl)tetrahydro-2H-pyran (0.128 mL, 0.974 mmol) and the reaction mixture was stirred at 100° C. under microwave irradiation for 45 min. The reaction mixture was diluted with water (10 mL) and, extracted with EtOAc (3×10 mL). The organic layer was washed with brine solution (10 mL), then passed through a hydrophobic frit and concentrated in vacuo to afford an orange oil. The resulting residue was dissolved in 3 mL DCM and was purified using a 12 g normal phase silica column, eluting with cyclohexane to 50% EtOAc (+1% NEt₃) in cyclohexane to give the title compound as a yellow oil (193 mg). LCMS (System A): t_{RET}=0.84 min; MH⁺ 323, 325, 327.

### Intermediate 26: rac-1-(3-((2,4-dibromo-1H-imidazol-1-yl)methyl)piperidin-1-yl)ethanone

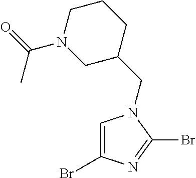 [SMILES: CC(=O)N1CCCC(Cn2cc(Br)nc2Br)C1]

1-(3-(bromomethyl)piperidin-1-yl)ethan-1-one (366 mg, 1.664 mmol), 2,4-dibromo-1H-imidazole (300 mg, 1.328 mmol) and potassium carbonate (556 mg, 4.02 mmol) were dissolved in acetonitrile (6 mL). The reaction was conducted under nitrogen and magnetic stirring at 80° C. for 17 h. The reaction mixture was filtered through Celite® and washed with ethyl acetate (20 mL). The solvent was then evaporated in vacuo to afford an orange oil. The residue was dissolved in 3 mL DCM and loaded onto a 40 g silica column. Eluting with EtOAc (+1% NEt₃) to 5% ethanol in EtOAc (+1% NEt₃) afforded the crude product. The residue was redissolved in 1:1 solution of MeOH:DMSO and purified by MDAP (Method C) to give the title compound as a colourless oil (162 mg). LCMS (System C): t_{RET}=0.74 min; MH⁺ 364, 366, 368.

### Example 1: 5-(1H-imidazol-2-yl)-1,3-dimethylpyridin-2(1H)-one

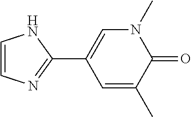

To a degassed solution of 2-bromo-1H-imidazole (21.0 g, 138 mmol), 1,3-dimethyl-5-(4,4,5,5-tetramethyl-1,3,2-dioxaborolan-2-yl)pyridin-2(1H)-one (commercially available from, for example, Milestone PharmaTech Inc, 38.0 g, 152 mmol) and potassium carbonate (57.4 g, 415 mmol) in 1,4-dioxane (200 mL) and water (60 mL) stirred under nitrogen at RT was added solid tetrakis (8.00 g, 6.92 mmol) in one charge. The reaction mixture was stirred at 100° C. for 16 h. The reaction mixture was filtered through a Celite® pad and the filterate was separated. The aqueous layer was re-extracted with 10% MeOH in DCM (2×100 mL). The combined organic layers were washed with brine solution (100 mL), dried over sodium sulphate, filtered and evaporated in vacuo to give the crude product as a brown gum. The crude product was triturated with 10% DCM in diethyl ether (2×50 mL). The resultant solid was filtered and dried under reduced pressure to afford crude compound as cream solid. This compound was triturated with diethylether and filtered through a Celite® pad and dried under reduced pressure to afford the title compound (23.0 g, 120 mmol, 87%) as cream solid. LCMS (System D): t_{RET}=2.14 min; MH⁺ 190.

### Example 2: 5-(4-bromo-1-ethyl-1H-imidazol-2-yl)-1,3-dimethylpyridin-2(1H)-one

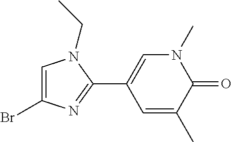 [SMILES: CCn1cc(Br)nc1-c1cc(C)c(=O)n(C)c1]

In a small flask, THF (5 mL) was added to tris(4-fluorophenyl)phosphine (0.797 g, 2.52 mmol) and diacetoxypalladium (0.283 g, 1.260 mmol), the resulting mixture was stirred for 5 min, then added to a 250 mL RB flask containing 2,4-dibromo-1-ethyl-1H-imidazole (for an example preparation, see Intermediate 1, 3.2 g, 12.60 mmol), potassium phosphate (8.03 g, 37.8 mmol) and 1,3-dimethyl-5-(4,4,5,5-tetramethyl-1,3,2-dioxaborolan-2-yl)pyridin-2(1H)-one (commercially available from, for example, Milestone PharmaTech, 10.1 g, 15.0 mmol). The resulting mixture was heated under reflux for 88 h. The reaction was allowed to cool down, then partitioned between EtOAc and water, the aqueous was extracted with EtOAc, the organics were combined and dried using Na₂SO₄, volatiles were removed under reduce pressure to afford an oil. The crude was purified by silica gel chromatography on a 100 g column using a 0-50% (3:1 (ethyl acetate:ethanol)) in ethyl acetate gradient over 10 CV. Relevant fractions were combined to afford the title compound (1.178 g, 3.98 mmol, 31.6%) as an oil. LCMS (System B): t_{RET}=0.73 min; MH⁺ 296, 298.

### Example 3: 5-(1-(cyclopropylmethyl)-1H-imidazol-2-yl)-1,3-dimethylpyridin-2(1H)-one

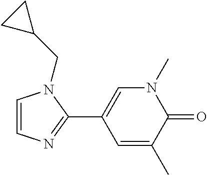 [SMILES: Cc1cc(-c2nccn2CC2CC2)cn(C)c1=O]

A mixture of 1,3-dimethyl-5-(4,4,5,5-tetramethyl-1,3,2-dioxaborolan-2-yl)pyridin-2(1H)-one (commercially available from, for example, Milestone PharmaTech, 50 mg, 0.2 mmol), 1-(cyclopropylmethyl)-2-iodo-1H-imidazole (for an example preparation, see Intermediate 2, 50 mg, 0.2 mmol), potassium carbonate (139 mg, 1.0 mmol) and bis(triphenylphosphine)palladium(II) chloride (14 mg, 10 mol %) in ethanol (2 mL) and toluene (2 mL) was heated in a microwave at 120° C. for 30 min. The cooled reaction mixture was diluted with ethyl acetate (25 mL) and filtered through Celite®. The solvent was evaporated from the filtrate and the residue chromatographed [0-10% ethanol/ethyl acetate] to give the title compound (10 mg, 0.041 mmol, 20%), as a colourless gum. LCMS (System A): t_{RET}=0.43 min; MH⁺ 244.

### Example 4: 5-(4-bromo-1-(cyclopropylmethyl)-1H-imidazol-2-yl)-1,3-dimethylpyridin-2(1H)-one

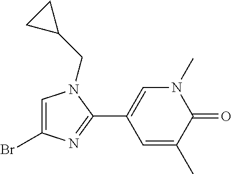 [SMILES: Cc1cc(-c2nc(Br)cn2CC2CC2)cn(C)c1=O]

A solution of 5-(1-(cyclopropylmethyl)-1H-imidazol-2-yl)-1,3-dimethylpyridin-2(1H)-one (for an example preparation, see Example 3, 33 mg, 0.123 mmol) in dichloromethane (2 mL) was cooled to 0° C. and treated with N-bromosuccinimide (22 mg, 0124 mmol). The reaction mixture was stirred at 0° C. for 1 h. The solvent was evaporated and the residue chromatographed [0-10% ethanol/ethyl acetate] to give the title compound (29 mg, 0.090 mmol, 73%), as a yellow oil. LCMS (System B): t_{RET}=0.84 min; MH⁺ 322, 324.

### Example 5: 5-(1-isobutyl-1H-imidazol-2-yl)-1,3-dimethylpyridin-2(1H)-one

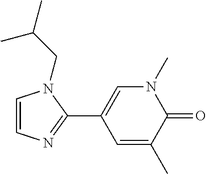 [SMILES: Cc1cc(-c2nccn2CC(C)C)cn(C)c1=O]

5-(1H-Imidazol-2-yl)-1,3-dimethylpyridin-2(1H)-one (for an example preparation, see Example 1, 0.114 g, 0.6 mmol) was dissolved in DMF (2.4 mL). 0.6 mL (0.15 mmol) of the solution was added to the 1-bromo-2-methyl propane (0.2 mmol). Potassium carbonate (0.041 g, 0.300 mmol) was added. The reaction vessel was sealed and left stirring for 18 h at 50° C. The temperature was increased to 70° C. After 2 h 2 eq. DIPEA (0.35 mL) was added to the reaction mixture along with a further 1 eq. of potassium carbonate (0.041 g, 0.300 mmol) and 1 eq. 1-bromo-2-methyl propane (0.2 mmol). The reaction was left stirring at 70° C. for 3 h. The reaction vessel was sealed and heated in a microwave using initial 600 W to 90° C. for 30 min. After cooling the reaction to RT the sample was purified by MDAP (Method B). The solvent was dried under a stream of nitrogen to give the title compound (20 mg, 0.081 mmol, 49%). LCMS (System A): t_{RET}=0.43 min; MH⁺ 246.

### Example 6: 1,3-dimethyl-5-(1-((tetrahydro-2H-pyran-4-yl)methyl)-1H-imidazol-2-yl)pyridin-2(1H)-one

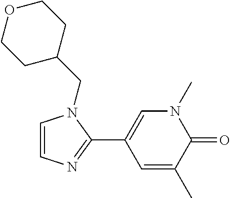 [SMILES: Cc1cc(-c2nccn2CC2CCOCC2)cn(C)c1=O]

5-(1H-Imidazol-2-yl)-1,3-dimethylpyridin-2(1H)-one (for an example preparation, see Example 1, 0.114 g, 0.6 mmol) was dissolved in DMF (2.4 mL). 0.6 mL (0.15 mmol) of the solution was added to 4-(bromomethyl)tetrahydro-2H-pyran (0.2 mmol). Potassium carbonate (0.041 g, 0.300 mmol) was added. The reaction vessel was sealed and left stirring for 18 h at 50° C. The temperature was increased to 70° C. After 2 h 2 eq. DIPEA (0.35 mL) was added to the reaction mixture along with a further 1 eq. of potassium carbonate (0.041 g, 0.30 mmol) and 1 eq. 4-(bromomethyl)tetrahydro-2H-pyran (0.2 mmol). The reaction was left stirring at 70° C. for 3 h. The reaction vessel was sealed and heated in a microwave using initial 600 W to 90° C. for 30 min. After cooling the reaction to RT the sample was purified by MDAP (Method B). The solvent was dried under a stream of nitrogen to give the title compound (8.3 mg, 0.029 mmol, 17%). LCMS (System A): t_{RET}=0.34 min; MH⁺ 288.

### Example 7: rac-1,3-dimethyl-5-(1-((tetrahydro-2H-pyran-2-yl)methyl)-1H-imidazol-2-yl)pyridin-2(1H)-one

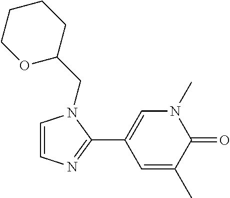 [SMILES: Cc1cc(-c2nccn2CC2CCCCO2)cn(C)c1=O]

5-(1H-Imidazol-2-yl)-1,3-dimethylpyridin-2(1H)-one (for an example preparation, see Example 1, 0.114 g, 0.6 mmol) was dissolved in DMF (2.4 mL). 0.6 mL (0.15 mmol) of the solution was added to rac-2-(bromomethyl)tetrahydro-2H-pyran (0.2 mmol). Potassium carbonate (0.041 g, 0.300 mmol) was added. The reaction vessel was sealed and left stirring for 18 h at 50° C. The temperature was increased to 70° C. After 2 h 2 eq. DIPEA (0.35 mL) was added to the reaction mixture along with a further 1 eq. of potassium carbonate (0.041 g, 0.30 mmol) and 1 eq. rac-2-(bromomethyl)tetrahydro-2H-pyran (0.2 mmol). The reaction was left stirring at 70° C. for 3 h. The reaction vessel was sealed and heated in a microwave using initial 600 W to 90° C. for 30 min. After cooling the reaction to RT the sample was purified by MDAP (Method B). The solvent was dried under a stream of nitrogen to give the title compound (5.7 mg, 0.029 mmol, 12%). LCMS (System A): t_{RET}=0.46 min; MH⁺ 288.

### Example 8: 1,3-dimethyl-5-(1-(piperidin-4-ylmethyl)-1H-imidazol-2-yl)pyridin-2(1H)-one

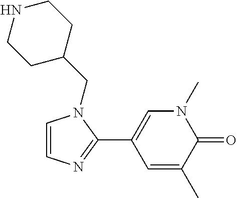 [SMILES: Cc1cc(-c2nccn2CC2CCNCC2)cn(C)c1=O]

5-(1H-Imidazol-2-yl)-1,3-dimethylpyridin-2(1H)-one (for an example preparation, see Example 1, 0.114 g, 0.6 mmol) was dissolved in DMF (2.4 mL). 0.6 mL (0.15 mmol) of the solution was added to tert-butyl 4-(bromomethyl)piperidine-1-carboxylate (0.2 mmol). Potassium carbonate (0.041 g, 0.300 mmol) and dimethyl sulfoxide (DMSO) (0.2 mL) added. The reaction vessel was sealed and heated in microwave using initial 600 W to 90° C. for 30 min. After cooling the reaction to RT, the sample in the reaction solvent (DMF, DMSO) was purified by MDAP (Method B). The solvent was dried under a stream of nitrogen to give the Boc-product. 0.5 mL 4M HCl in 1,4-dioxane was added and the sample left overnight. The solvent was removed. The sample was dissolved in DMSO (0.8 mL) and purified by MDAP (Method B). The solvent was dried under a stream of nitrogen to give the title compound (3.9 mg). LCMS (System B): t_{RET}=0.56 min; MH⁺ 286.

### Example 9: rac-1,3-dimethyl-5-(1-((tetrahydrofuran-2-yl)methyl)-1H-imidazol-2-yl)pyridin-2(1H)-one

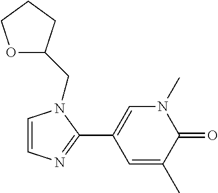 [SMILES: Cc1cc(-c2nccn2CC2CCCO2)cn(C)c1=O]

Sodium hydride (80 mg, 2 mmol) and 5-(1H-imidazol-2-yl)-1,3-dimethylpyridin-2(1H)-one (for an example preparation, see Example 1, 0.284 g, 1.5 mmol) were dissolved in DMF (6 mL) and the mixture stirred at 22° C. for 15 min. 0.6 mL of the mixture was then added to 2-(bromomethyl)tetrahydrofuran (0.15 mmol). The reaction vessel was sealed and left stirring at 22° C. for 18 h. After 18 h, a further equivalent of sodium hydride (0.008 g, 0.20 mmol) was added to the reaction and the reaction was left stirring for 2 h at 22° C. The reaction was quenched with 0.3 mL MeOH. The sample was purified by MDAP (Method B). The solvent was dried under a stream of nitrogen to give the title compound (3.2 mg, 0.012 mmol, 7%). LCMS (System B): t_{RET}=0.64 min; MH⁺ 274.

### Example 10: 5-(1-(2-methoxyethyl)-1H-imidazol-2-yl)-1,3-dimethylpyridin-2(1H)-one

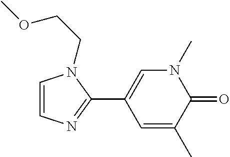 [SMILES: COCCn1ccnc1-c1cc(C)c(=O)n(C)c1]

Sodium hydride (0.053 g, 1.32 mmol) and 5-(1H-imidazol-2-yl)-1,3-dimethylpyridin-2(1H)-one (for an example preparation, see Example 1, 0.114 g, 0.6 mmol) was dissolved in DMF (2.4 mL) and the mixture stirred at 22° C. for 15 min. 0.6 mL of the mixture (0.15 mmol core, 0.33 mmol sodium hydride) was then added to 1-bromo-2-methoxyethane (0.2 mmol). The reaction vessel was sealed and left stirring at 22° C. for 18 h. The reaction was quenched with 0.3 mL MeOH. The sample in DMF/MeOH was purified by MDAP (Method B). The solvent was dried under a stream of nitrogen to give the title compound (5.4 mg, 13%). LCMS (System A): t_{RET}=0.27 min; MH⁺ 248.

### Example 11: 5-(1-(1,3-dimethoxypropan-2-yl)-1H-imidazol-2-yl)-1,3-dimethylpyridin-2(1H)-one

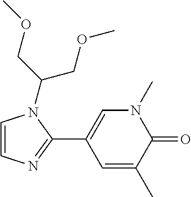 [SMILES: COCC(COC)n1ccnc1-c1cc(C)c(=O)n(C)c1]

DIAD (0.057 mL, 0.291 mmol) was added in the dark to a stirred solution of 5-(1H-imidazol-2-yl)-1,3-dimethylpyridin-2(1H)-one (for an example preparation, see Example 1, 50 mg, 0.264 mmol), 1,3-dimethoxypropan-2-ol (0.035 mL, 0.291 mmol) and triphenylphosphine (76 mg, 0.291 mmol) in dry THF (0.5 mL). The reaction was stirred at RT overnight under an atmosphere of nitrogen. A further 2 eq of 1,3-dimethoxypropan-2-ol (0.063 mL, 0.529 mmol) and triphenylphosphine (139 mg, 0.529 mmol) were added, the reaction mixture purged with nitrogen for 5 min prior to the addition of 2 eq DIAD (0.103 mL, 0.529 mmol). After stirring at 40° C. for 5 h, conversion was still limited so a further 2 eq 1,3-dimethoxypropan-2-ol (0.063 mL, 0.529 mmol) and 2 eq DIAD (0.103 mL, 0.529 mmol) were added, the reaction heated in the microwave for 1 h at 50° C. The solvent was removed under reduced pressure, and the residue dissolved in 1:1 MeCN:DMSO (6 mL) and purified by 2×MDAP (Method B). The solvent was dried under a stream of nitrogen and the product containing fractions combined. The samples were dissolved in 1:1 MeCN:DMSO (0.9 mL) and purified by MDPA (Method B). The product containing fractions were once more collated and dried, with impurities still in evidence. After an aqueous extraction also failed to remove the impurities, the sample was submitted for further purification. 5 mgs of material as dissolved in DMSO (3 mL). 3000 μL injections were made onto a CSH C18 150×30 mm, 5 μm column which was eluted using a gradient of 0-99% MeCN in 10 mM aqueous ammonium bicarbonate (adjusted to pH 10 with ammonia) at 40 mL/min over 41 min. After evaporation, the title compound was obtained as a white solid 2 mg. LCMS (System B): t_{RET}=0.66 min; MH⁺ 292.

### Example 12: methyl 2-(1,5-dimethyl-6-oxo-1,6-dihydropyridin-3-yl)-1H-imidazole-5-carboxylate

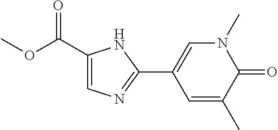 [SMILES: COC(=O)c1cnc(-c2cc(C)c(=O)n(C)c2)[nH]1]

Methyl 2-(1,5-dimethyl-6-oxo-1,6-dihydropyridin-3-yl)-1-((2-(trimethylsilyl)ethoxy)methyl)-1H-imidazole-4-carboxylate (for an example preparation, see Intermediate 8, 1.76 g, 4.66 mmol) was added to a round bottomed flask containing a stirrer bar and dissolved in anhydrous methanol (20 mL). The flask was purged with nitrogen by evacuation-refill, and trimethylsilylchloride (11.92 mL, 93 mmol) added to the reaction mixture. The reaction mixture was stirred at 40° C. for 18 h under an atmosphere of nitrogen. The solvent was removed under reduced pressure, and the crude product twice redissolved in methanol (30 mL) and the solvent removed in vacuo. The crude product was loaded in methanol and purified by SPE using a 20 g sulphonic acid (SCX) cartridge, with sequential solvent elution of methanol followed by 2M ammonia in methanol (2 M). The appropriate fractions were combined and the solvent removed in vacuo to give the title compound as a white solid, (773 mg, 3.13 mmol, 67%). LCMS (System B): t_{RET}=0.56 min; MH⁺ 248.

### Example 13: 5-(5-chloro-1H-imidazol-2-yl)-1,3-dimethylpyridin-2(1H)-one

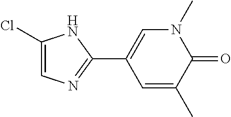

5-(4-Chloro-1-((2-(trimethylsilyl)ethoxy)methyl)-1H-imidazol-2-yl)-1,3-dimethylpyridin-2(1H)-one (for an example preparation, see Intermediate 5, 538 mg, 1.44 mmol) was added to a round bottomed flask containing a stirrer bar and dissolved in anhydrous methanol (6 mL). The flask was purged by evacuation-refill, and TMS-Cl (3.8 mL, 29.7 mmol) was added to the reaction mixture. The reaction mixture was stirred at 40° C. overnight. Another portion of TMS-Cl (3.8 mL, 29.7 mmol) was added to the reaction mixture and the reaction was left to stir at 40° C. overnight. The solvent was removed under reduced pressure. To remove any residual impurities, and to yield the product as a free base rather than a salt, the crude product was loaded in methanol and purified by SPE on a sulphonic acid (SCX) 2 g cartridge with sequential solvent elution using methanol, 2M ammonia/methanol. The appropriate fractions were combined and evaporated in vacuo to give the title compound (324 mq). LCMS (System A): t_{RET}=0.57 min; MH⁺ 224, 226.

### Example 14: 2-(1,5-dimethyl-6-oxo-1,6-dihydropyridin-3-yl)-1H-imidazole-5-carboxamide

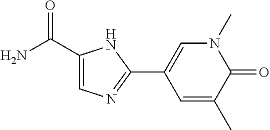 [SMILES: Cc1cc(-c2ncc(C(N)=O)[nH]2)cn(C)c1=O]

2-(1,5-dimethyl-6-oxo-1,6-dihydropyridin-3-yl)-1-((2-(trimethylsilyl)ethoxy)methyl)-1H-imidazole-4-carbonitrile (for an example preparation, see Intermediate 11, 848 mg, 2.462 mmol) was added to a round bottomed flask containing a stirrer bar and dissolved in anhydrous methanol (10 mL). The flask was purged by evacuation-refill, and TMS-Cl (6.29 mL, 49.2 mmol) was added to the reaction mixture. The reaction mixture was stirred at 40° C. overnight under an atmosphere of nitrogen. Subsequent addition of methanol (2×30 mL) and repeated solvent evaporation was used to try and ensure removal of any high boiling by-products. The crude product was dissolved in methanol and purified by SPE on 20 g sulphonic acid (SCX) cartridge with sequential solvent elution using methanol, 2M ammonia/methanol. Product containing fractions were combined and the solvent removed under reduced pressure. The sample was partially dissolved in 3 mL MeOH:DMSO and filtered. The sample was purified by MDAP (Method B). The solvent was dried under a stream of nitrogen to give two batches of product with distinct identities. The residue from the initial filtration was dissolved in H₂O (+minimum 2M HCl) 6 mL and purified by MDAP (Method C). The solvent was dried under a stream of nitrogen give 3 batches of product with distinct identities. Comparable product fractions were combined from across the 3 runs. The product was obtained as a white solid 290 mg. 20 mg was dissolved in 1:1 MeOH:DMSO (0.9 mL) and purified by MDAP (Method B). The solvent was removed under a stream of nitrogen to give the title compound as a white solid (14 mg). LCMS (System B): t_{RET}=0.44 min; MH⁺ 233.

### Example 15: 2-(1,5-dimethyl-6-oxo-1,6-dihydropyridin-3-yl)-1H-imidazole-4,5-dicarbonitrile

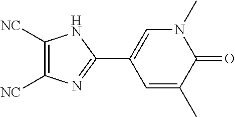 [SMILES: Cc1cc(-c2nc(C#N)c(C#N)[nH]2)cn(C)c1=O]

1,3-Dimethyl-5-(4,4,5,5-tetramethyl-1,3,2-dioxaborolan-2-yl)pyridin-2(1H)-one (1472 mg, 5.91 mmol), 2-bromo-1H-imidazole-4,5-dicarbonitrile (776 mg, 3.94 mmol), and potassium carbonate (1361 mg, 9.85 mmol) were added to a 5 mL microwave vial containing a stirrer bar. 1,4-Dioxane (15 mL) and water (5 mL) were added to the vial, which was purged with nitrogen for 5 min prior to the addition of tetrakis(triphenylphosphine)palladium(0) (137 mg, 0.118 mmol). After a further 5 min purge with nitrogen, the vial was capped and heated in the microwave at 110° C. for 1 h. The mixture was filtered through Celite® and the solvent removed under reduced pressure. The residue was stirred to form a suspension in ethyl acetate, then filtered through Celite® and washed with further ethyl acetate. The product, of low solubility, was rinsed through the cartridge with methanol into a separate round bottomed flask. Inorganic base remained present, attempted purification via adhesion to porelite polymer and subsequent washing was unsuccessful. The fractions were dissolved in methanol, filtered to remove any porelite, and the solvent removed under reduced pressure, filtered, and the filtrate dried to yield a preliminary batch of product (101 mg). With product still in evidence in the filter cake, this was suspended in ethanol and filtered, washing with further ethanol to isolate further product. Removing the solvent from the filtrate yielded a second, larger batch of the title compound (885 mg). LCMS (System B): t_{RET}=0.57 min; MH⁺ 240.

### Example 16: 5-(1-(1,3-dimethoxypropan-2-yl)-4,5-dimethyl-1H-imidazol-2-yl)-1,3-dimethylpyridin-2(1H)-one

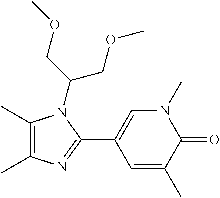 [SMILES: COCC(COC)n1c(-c2cc(C)c(=O)n(C)c2)nc(C)c1C]

A mixture of diacetyl (50 mg, 0.581 mmol), 1,3-dimethoxypropan-2-amine (83 mg, 0.697 mmol), ammonium acetate (53.7 mg, 0.697 mmol), 1,5-dimethyl-6-oxo-1,6-dihydropyridine-3-carbaldehyde (88 mg, 0.581 mmol) and acetic acid (0.166 mL, 2.90 mmol) were taken up in chloroform (0.2 mL). The reaction vessel was sealed and heated in microwave reactor to 140° C. for 10 min. The sample was injected as is and purified by MDAP (Method B). The solvent was dried under a stream of nitrogen to give the title compound (16 mg). LCMS (System A): t_{RET}=0.42 min; MH⁺ 320.

### Example 17: 5-(4-(4-bromophenyl)-1-(1,3-dimethoxypropan-2-yl)-1H-imidazol-2-yl)-1,3-dimethylpyridin-2(1H)-one

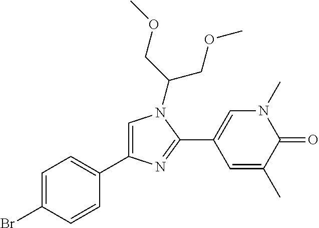 [SMILES: COCC(COC)n1cc(-c2ccc(Br)cc2)nc1-c1cc(C)c(=O)n(C)c1]

A mixture of ammonium acetate (38 mg, 0.493 mmol), 2-bromo-1-(4-bromophenyl)ethanone (92 mg, 0.331 mmol), 1,5-dimethyl-6-oxo-1,6-dihydropyridine-3-carbaldehyde (50 mg, 0.331 mmol), 1,3-dimethoxypropan-2-amine (42.3 μL, 0.331 mmol) were placed into a 4 mL glass vial dissolved in chloroform (0.2 mL) and acetic acid (50 μL, 0.873 mmol) was added. The reaction vessel was sealed and heated in microwave reactor to 130° C. for 10 min. The sample diluted with DMSO (1 mL), split into two injections (approx 0.7 mL each) and purified by MDAP (Method B). The solvent was dried under a stream of nitrogen to give the product. The sample was dissolved in DMSO (0.6 mL) and purified by MDAP (Method A). The solvent was dried under a stream of nitrogen to give the title compound as a white solid (8.8 mg). LCMS (System A): t_{RET}=0.84 min; MH⁺ 446, 448.

### Example 18 & 19: rac-5-(4-chloro-1-((tetrahydro-2H-pyran-2-yl)methyl)-1H-imidazol-2-yl)-1,3-dimethylpyridin-2(1H)-one (Example 18) & rac-5-(5-chloro-1-((tetrahydro-2H-pyran-2-yl)methyl)-1H-imidazol-2-yl)-1,3-dimethylpyridin-2(1H)-one (Example 19)

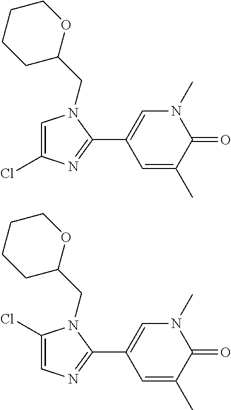 [SMILES: Cc1cc(-c2nc(Cl)cn2CC2CCCCO2)cn(C)c1=O ; Cc1cc(-c2ncc(Cl)n2CC2CCCCO2)cn(C)c1=O]

To a stirred solution of 5-(4-chloro-1H-imidazol-2-yl)-1,3-dimethylpyridin-2(1H)-one (for an example preparation, see Example 13, 40 mg, 0.161 mmol) in DMF (2 mL) at 0° C. was added sodium hydride (12.88 mg, 0.322 mmol). The reaction was stirred for 30 min, then 2-(bromomethyl)tetrahydro-2H-pyran (0.021 mL, 0.161 mmol) was added. The reaction was allowed to warm to RT and left to stir at RT overnight. The solvent was evaporated in vacuo. The solid was dissolved in DMF (0.8 mL) and transferred to a microwave vial. Potassium carbonate (44.5 mg, 0.322 mmol), 2-(bromomethyl)tetrahydro-2H-pyran (0.062 mL, 0.483 mmol) and DIPEA (0.056 mL, 0.322 mmol) were added. The reaction vessel was sealed and heated to 100° C. for 2 h. The reaction was left to stir overnight. The solvent evaporated in vacuo. The sample was dissolved in 1:1 MeOH:DMSO (1 mL) and purified by MDAP (Formic). Both isomers were collected and kept separate. The solvent was evaporated in vacuo and further dried under a stream of nitrogen. The major isomer was dissolved in MeOH and added to an SCX column and eluted with MeOH followed by 2M ammonia in MeOH. The appropriate fractions were evaporated in vacuo and further dried under a stream of nitrogen. The sample was dissolved in 1 mL MeOH and purified by MDAP (High pH). The solvent was dried under a stream of nitrogen to give the title compound (Example 18) (4.6 mg). LCMS (System A): t_{RET}=0.87 min; MH⁺ 322, 324. The minor isomer was dissolved in MeOH and added to an SCX column and eluted with MeOH followed by 2M ammonia in MeOH. The appropriate fractions were evaporated in vacuo and further dried under a stream of nitrogen to give the title compound (Example 19) (4 mg). LCMS (System A): t_{RET}=0.70 min; MH⁺ 322.

### Example 20: 5-(5-chloro-1-((tetrahydro-2H-pyran-4-yl)methyl)-1H-imidazol-2-yl)-1,3-dimethylpyridin-2(1H)-one

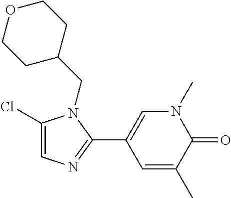 [SMILES: Cc1cc(-c2ncc(Cl)n2CC2CCOCC2)cn(C)c1=O]

To a stirred solution of 5-(4-chloro-1H-imidazol-2-yl)-1,3-dimethylpyridin-2(1H)-one (for an example preparation, see Example 13, 40 mg, 0.179 mmol) in DMF (2 mL) at 0° C. was added sodium hydride (17.88 mg, 0.447 mmol). The reaction was stirred for 30 min, then 4-(bromomethyl)tetrahydro-2H-pyran (0.035 mL, 0.268 mmol) was added. The reaction was allowed to warm to RT and stirred for a further 18 h. The reaction was quenched with methanol (2 mL) and the solvent removed in vacuo. The reaction mixture was redissolved in DMF (2 mL) in a 2-5 mL microwave vial, and 4-(bromomethyl)tetrahydro-2H-pyran (0.071 mL, 0.537 mmol), potassium carbonate (49.4 mg, 0.358 mmol) and DIPEA (0.062 mL, 0.358 mmol) were added and the reaction heated to 100° C. for 18 h. The solvent was removed in vacuo and the crude residue dissolved in DMSO/MeOH (1.8 mL). Purification by MDAP (High pH) afforded the title compound (3.8 mg, 10.63 μmol, 6%) as a colourless film. LCMS (System A): t_{RET}=0.56 min; MH⁺ 322, 324. The other isomer (Example 30) was also isolated as a colourless film (22 mg).

### Example 21: rac-5-(5-chloro-1-((tetrahydro-2H-pyran-3-yl)methyl)-1H-imidazol-2-yl)-1,3-dimethylpyridin-2(1H)-one

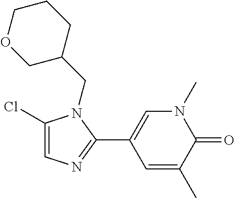 [SMILES: Cc1cc(-c2ncc(Cl)n2CC2CCCOC2)cn(C)c1=O]

To a stirred solution of 5-(4-chloro-1H-imidazol-2-yl)-1,3-dimethylpyridin-2(1H)-one (for an example preparation, see Example 13, 40 mg, 0.179 mmol) in DMF (2 mL) at 0° C. was added sodium hydride (17.88 mg, 0.447 mmol). The reaction was stirred for 30 min, then 3-(bromomethyl)tetrahydro-2H-pyran (0.034 mL, 0.268 mmol) was added. The reaction was allowed to warm to RT and stirred for a further 18 h. The reaction was quenched with methanol (2 mL) and the solvent removed in vacuo. The reaction was redissolved in DMF (2 mL) in a 2-5 mL microwave vial, and DIPEA (0.062 mL, 0.358 mmol), potassium carbonate (49.4 mg, 0.358 mmol) and 3-(bromomethyl)tetrahydro-2H-pyran (0.067 mL, 0.537 mmol) added and the reaction heated to 100° C. for 18 h. The solvent was removed in vacuo and the crude residue redissolved in DMSO/MeOH (1.8 mL) and filtered. The solution was purified by MDAP (Method A) to afford the product (4.4 mg, 0.012 mmol, 7%) as a colourless film. LCMS (System A): t_{RET}=0.59 min; MH⁺ 322, 324. The other isomer (Example 27) could also be isolated as a colourless film (20 mg).

### Example 22: 5-(4-chloro-1-(1,3-dimethoxypropan-2-yl)-1H-imidazol-2-yl)-1,3-dimethylpyridin-2(1H)-one

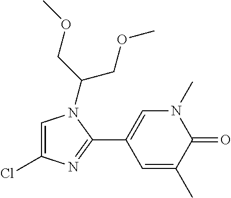 [SMILES: COCC(COC)n1cc(Cl)nc1-c1cc(C)c(=O)n(C)c1]

1,3-Dimethyl-5-(4,4,5,5-tetramethyl-1,3,2-dioxaborolan-2-yl)pyridin-2(1H)-one (73.8 mg, 0.296 mmol), 2-bromo-4-chloro-1-(1,3-dimethoxypropan-2-yl)-1H-imidazole (for an example preparation, see Intermediate 13, 56 mg, 0.197 mmol), and potassium carbonate (68.2 mg, 0.494 mmol) were added to a 5 mL microwave vial containing a stirrer bar. 1,4-Dioxane (0.75 mL) and methanol (0.25 mL) were added to the vial, which was purged with nitrogen for 5 min prior to the addition of tetrakis(triphenylphosphine)palladium(0) (6.85 mg, 5.92 μmol). After a further 5 min purge with nitrogen, the vial was capped and heated in the microwave at 100° C. for 1 h. The solvent was removed under reduced pressure and the residue taken up in EtOAc (20 mL). The solution was filtered through Celite® and the solvent removed from the filtrate under reduced pressure. The samples were dissolved in 1:1 MeOH:DMSO (0.9 mL) and purified by MDAP (High pH). The solvent was dried under a stream of nitrogen to give the crude product. Further purification was attempted; the sample was loaded in iPrOH and purified by SPE on 1 g sulphonic acid (SCX) cartridge using a sequential solvents iPrOH, 2M ammonia/iPrOH. This failed to remove the 3% impurity, and the fractions were recombined, the solvent removed under reduced pressure. The sample (ca. 60 mg) was dissolved in 12 mL DMSO. 3000 μL injections were made onto a CSH C18 150×30 mm, 5 μm column using a gradient of 15-99% MeCN in aqueous ammonium bicarbonate (adjusted to pH 10 with ammonia). The pure fractions were combined and blown down under a stream of nitrogen at RT in the dark, so as to remove the MeCN. The residual aqueous mixture was attached to a rotary evaporator (without a vacuum) and spun in a bath of acetone and solid CO₂ for 30 minutes in the dark, so as to get as thin a film of ice within the Florentine flask as possible. The flask containing the frozen mixture was covered with foil and lyophilised overnight to give a colourless solid. This solid was transferred to a pre-weighed vial using a volatile solvent (4×DCM; 15 mL) to avoid warming during evaporation. The solvent was removed by nitrogen blow-down at RT and the residual amorphous foam was redissolved in DCM (ca. 3 mL) and precipitated with n-hexane (ca. 12 mL). The solvents were removed by nitrogen blow-down at RT and evaporation was continued overnight to give the title compound as an amorphous and colourless solid (40 mg). LCMS (System B): t_{RET}=0.82 min; MH⁺ 326, 328. ¹H NMR (CD₃OD, 400 MHz) δ: 7.82 (d, 1H), 7.57 (m, 1H), 7.37 (s, 1H), 4.53 (m, 1H), 3.72-3.63 (m, 4H), 3.61 (s, 3H), 3.32 (1H, m), 3.31 (s, 6H), 2.15 (s, 3H).

### Example 23: rac-5-(1-((1-acetylpiperidin-3-yl)methyl)-5-chloro-1H-imidazol-2-yl)-1,3-dimethylpyridin-2(1H)-one

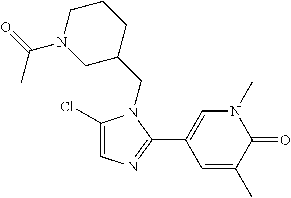 [SMILES: CC(=O)N1CCCC(Cn2c(Cl)cnc2-c2cc(C)c(=O)n(C)c2)C1]

5-(4-chloro-1H-imidazol-2-yl)-1,3-dimethylpyridin-2(1H)-one (for an example preparation, see Example 13, 150 mg, 0.671 mmol) was dissolved in DMF (4 mL) in a microwave vial containing a stirrer bar, and purged with nitrogen for 10 min. 1-(3-(Bromomethyl)piperidin-1-yl)ethanone (221 mg, 1.006 mmol) was added and the solution heated to 80° C. The reaction mixture was stirred overnight under an atmosphere of nitrogen. Further 1-(3-(bromomethyl)piperidin-1-yl)ethanone (59.0 mg, 0.268 mmol) was added to the reaction mixture, which was stirred for a further 7 h at 80° C. The solvent was removed under reduced pressure and the residue redissolved in EtOAc. The solution was filtered through Celite® and dissolved in 1:1 MeOH:DMSO (3 mL) and purified by MDAP (Method C). The solvent was evaporated in vacuo to give the title compound as a pale yellow oil (35 mg). LCMS (System B): t_{RET}=0.72 min; MH⁺ 363, 365. The other isomer (Example 24) could also be isolated as a pale yellow oil (116 mg).

### Example 24: rac-5-(1-((1-acetylpiperidin-3-yl)methyl)-4-chloro-1H-imidazol-2-yl)-1,3-dimethylpyridin-2(1H)-one

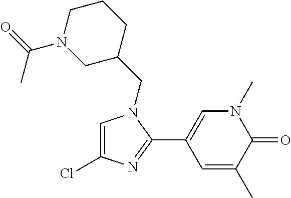 [SMILES: CC(=O)N1CCCC(Cn2cc(Cl)nc2-c2cc(C)c(=O)n(C)c2)C1]

5-(4-chloro-1-(piperidin-3-ylmethyl)-1H-imidazol-2-yl)-1,3-dimethylpyridin-2(1H)-one (for an example preparation, see Intermediate 17, 2.348 g, 7.32 mmol) was taken up in DCM (35 mL). Et₃N (3.06 mL, 21.96 mmol) was added followed by AcCl (0.781 mL, 10.98 mmol) and stirred at RT for 30 mins. The reaction was quenched with sat. NaHCO₃ (50 mL) and stirred for 10 mins. The organic layer was extracted and filtered through a hydrophobic frit then concentrated in vacuo to give crude title compound as an orange foam. The crude product was applied to a 100 g silica cartridge in the minimum of DCM and eluted with 0.5% 2M NH₃ in methanol in DCM for 2CV then 0.5-8% 2M NH₃ in MeOH over 10CV then held at 8% for 5CV. The appropriate fractions were concentrated in vacuo to give the title compound (2.3422 g) as a cream solid after co-evaporating in vacuo with Et₂O. LCMS (System B): t_{RET}=0.72 min; MH⁺ 363, 365. ¹H NMR (CDCl₃, 400 MHz): δ 7.44 (1H, d), 7.33-7.34 (1H, m), 6.91 (1H, s), 4.15-4.20 (1H, m), 3.84-3.93 (1H, m), 3.71-3.77 (1H, m), 3.62-3.66 (4H, m), 3.11-3.18 (1H, m), 2.67-2.73 (1H, m), 2.21 (3H, s), 2.08 (3H, s), 1.88-1.95 (1H, m), 1.64-1.77 (2H, m), 1.43-1.51 (2H, m), 1.13-1.20 (1H, m).

### Examples 25 and 26: 5-(1-((1-acetylpiperidin-3-yl)methyl)-4-chloro-1H-imidazol-2-yl)-1,3-dimethylpyridin-2(1H)-one single enantiomers

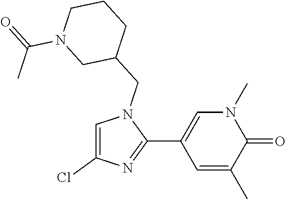 [SMILES: CC(=O)N1CCCC(Cn2cc(Cl)nc2-c2cc(C)c(=O)n(C)c2)C1]

rac-5-(1-((1-Acetylpiperidin-3-yl)methyl)-4-chloro-1H-imidazol-2-yl)-1,3-dimethylpyridin-2(1H)-one (approx 2.3 g) was purified by preparative chiral HPLC using a 2 cm×25 cm Chiralpak IB (10 μm) column. Approx 2.3 g of material was purified with ˜100 mg of material dissolved in 2 mL EtOH at a time. 1 mL of the solution was injected onto the column at a time and run with 20% EtOH/heptane, flow rate=20 mL/min, wavelength 215 nm. Fractions from 10.5-12 min (enantiomer 1), 12-13.5 min (mixed) and from 13.5-17.5 min (enantiomer 2) were bulked and evaporated to give Example 25 (enantiomer 1, 1.06 g, >99.5% chiral purity) and Example 26 (enantiomer 2, 830 mg, >99.5% chiral purity). Chiral purity was confirmed by analytical chiral HPLC was using a 4.6 mmid×25 cm Chiralpak IB column run with 20% EtOH/heptane, flow rate=1.0 mL/min, wavelength 215 nm; enantiomer 1 t_{RET}˜17 min, enantiomer 2 t_{RET}˜19 min.

### Example 27: rac-5-(4-chloro-1-((tetrahydro-2H-pyran-3-yl)methyl)-1H-imidazol-2-yl)-1,3-dimethylpyridin-2(1H)-one

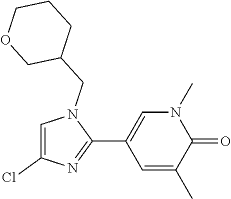 [SMILES: Cc1cc(-c2nc(Cl)cn2CC2CCCOC2)cn(C)c1=O]

A degassed mixture of rac-2-bromo-4-chloro-1-((tetrahydro-2H-pyran-3-yl)methyl)-1H-imidazole (for an example preparation, see Intermediate 19, 9.1 g, 32.6 mmol), 1,3-dimethyl-5-(4,4,5,5-tetramethyl-1,3,2-dioxaborolan-2-yl)pyridin-2(1H)-one (commercially available from, for example, Milestone PharmaTech, 12 g, 48.2 mmol), potassium carbonate (13.50 g, 98 mmol) and tetrakis(triphenylphosphine)palladium(0) (1.00 g, 0.865 mmol) in 1,4-dioxane (66 mL) and water (22.00 mL) was stirred at reflux under a nitrogen atmosphere for 20 h. The reaction mixture was filtered through a pad of Celite® and the cake washed with ethyl acetate (50 mL). The combined filtrates were concentrated in vacuo and the residue partitioned between ethyl acetate (200 mL) and water (200 mL). The organic phase was separated and the aqueous phase (an emulsion) was back extracted with ethyl acetate (2×150 mL). The combined organic extracts were washed with brine (200 mL), dried (MgSO₄), filtered and concentrated a brown gum (16.0 g). The gum was dissolved in ethyl acetate and purified on a silica cartridge (330 g) using a 0-30% ethanol-ethyl acetate+1% Et₃N gradient over 12 CV. The appropriate fractions were combined and evaporated in vacuo to give beige sticky foam (8.58 g). This gum was triturated with TBME (˜100 mL). The resulting suspension was filtered and the off-white solid dried in vacuo to furnish the title compound (7.16 g, 68%). LCMS (System B): t_{RET}=0.77 min; MH⁺ 322, 324. The mother liquors from the trituration were concentrated in vacuo to give a brown oil. The oil was dissolved in ethyl acetate and purified on a silica cartridge (80 g) using a 0-30% ethanol+1% Et₃N-ethyl acetate gradient over 12 CV. The appropriate fractions were combined and evaporated in vacuo and the resulting foam triturated with TBME (˜15 mL). The resulting suspension was filtered and the solid dried in vacuo to furnish a further batch of the title compound (485 mg, 5%) as an off-white solid. LCMS (System B): t_{RET}=0.77 min; MH⁺ 322, 324. ¹H NMR (400 MHz, METHANOL-d₄) δ 7.84 (m, 1H), 7.58 (m, 1H), 7.24 (s, 1H), 4.01-4.09 (m, 1H), 3.89-3.97 (m, 1H), 3.68-3.76 (m, 1H), 3.59-3.67 (m, 4H), 3.50 (m, 1H), 3.21 (dd, J=7.7, 11.4 Hz, 1H), 2.19 (s, 3H), 2.03 (m, 1H), 1.67-1.77 (m, 1H), 1.57-1.67 (m, 1H), 1.52 (m, 1H), 1.24-1.35 (m, 1H).

### Examples 28 and 29: 5-(4-chloro-1-((tetrahydro-2H-pyran-3-yl)methyl)-1H-imidazol-2-yl)-1,3-dimethylpyridin-2(1H)-one single enantiomers

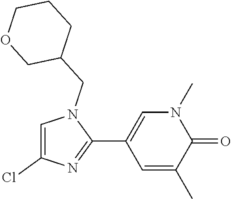 [SMILES: Cc1cc(-c2nc(Cl)cn2CC2CCCOC2)cn(C)c1=O]

rac-5-(4-Chloro-1-((tetrahydro-2H-pyran-3-yl)methyl)-1H-imidazol-2-yl)-1,3-dimethylpyridin-2(1H)-one (for an example preparation, see Example 27, 1 g) was dissolved in ethanol (10 mL) and was subjected to chiral preparative chromatography using a Chiralpak AD-H (250×30 mm) column. 250 μL of solution was injected on to the column at a time and run with 85% heptane (+0.2% v/v isopropylamine) and 15% ethanol (+0.2% v/v isopropylamine), flow rate=42.5 mL/min (45 bar), UV Diode Array at 280 nm. Fractions containing the first eluting isomer were collected between 18.2 min and 20.7 min. Fractions containing the second eluting isomer were collected between 21.7 min and 26 min. The combined isomer fractions were evaporated to dryness to give Example 29 (enantiomer 1, 431 mg, 99.9% chiral purity) and Example 30 (enantiomer 2, 447 mg, 97.3% chiral purity). Chiral purity was confirmed by analytical chiral HPLC was using a Chiralpak AD-H 250×4.6 mm column run with heptane:EtOH:isopropylamine 85:15:0.2, flow rate=1 mL/min, wavelength 250 nM; enantiomer 1 t_{RET}˜20 min, enantiomer 2 t_{RET}˜23.5 min.

### Example 30: 5-(4-chloro-1-((tetrahydro-2H-pyran-4-yl)methyl)-1H-imidazol-2-yl)-1,3-dimethylpyridin-2(1H)-one

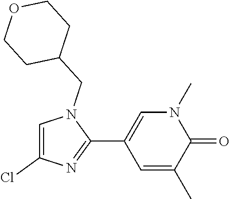 [SMILES: Cc1cc(-c2nc(Cl)cn2CC2CCOCC2)cn(C)c1=O]

A stirred, degassed mixture of 2-bromo-4-chloro-1-((tetrahydro-2H-pyran-4-yl)methyl)-1H-imidazole (for an example preparation, see Intermediate 21, 29.8 g, 107 mmol), 1,3-dimethyl-5-(4,4,5,5-tetramethyl-1,3,2-dioxaborolan-2-yl)pyridin-2(1H)-one (commercially available from, for example, Milestone Pharmatech, 31.9 g, 128 mmol), potassium carbonate (44.2 g, 320 mmol) and tetrakis(triphenylphosphine)palladium(0) (0.616 g, 0.533 mmol) in 3:1 1,4-dioxane:water (280 mL) was heated at reflux for 20 h. The reaction mixture was filtered through a plug of Celite®, the filter cake was washed with ethyl acetate (100 mL). The combined filtrates were concentrated in vacuo and the residue partitioned between ethyl acetate (500 mL) and water (500 mL). The organic phase was separated and the aqueous phase back extracted with ethyl acetate (2×300 mL). The combined organic phases were concentrated in vacuo to give the crude product (39.9 g). The crude product was dissolved in 10% MeOH in ethyl acetate and purified on a silica cartridge (750 g) using a 0-25% ethanol-ethyl acetate+1% Et₃N gradient over 15 CV. The appropriate fractions were combined and concentrated in vacuo and azeotroped with TBME to give a very pale yellow solid (Batch 1, 22.5 g) and a red oil (Batch 2, 5.1 g). Batch 1 was triturated with TBME (˜300 mL), filtered and the solid was dried in vacuo to give an off-white solid (Batch 3, 21.39 g). The filtrate was concentrated in vacuo to furnish Batch 4 (1.2 g). Batches 2 and 4 were combined, dissolved in ethyl acetate and purified on a silica cartridge (330 g) using a 0-25% ethanol+1% Et₃N gradient over 12 CV. The appropriate fractions were combined and evaporated in vacuo to give a red gum. This gum was triturated with TBME, filtered and the solid dried in vacuo to give an off-white solid (Batch 5, 3.25 g). The filtrate from this batch was concentrated in vacuo and triturated with TBME, filtered and the solid dried in vacuo to furnish the title compound as an off-white solid (Batch 6, 0.637 g). Batches 3 and 5 were combined and dissolved in methanol (500 mL) and treated with SiliaMetS Thiol (44.4 g, 53.3 mmol). The resulting mixture was stirred at 50° C. for 2 h. After cooling, the suspension was filtered through Celite® and the filtrate concentrated in vacuo to give a yellow gum. This gum was triturated with TBME (˜500 mL), the solid was collected by filtration. The filter cake was washed with TBME (100 mL) and dried in vacuo for 10 days to furnish the title compound (Batch 7, 21.04 g). LCMS (System B): t_{RET}=0.74 min; MH⁺ 322, 324. ¹H NMR (DMSO-d₆, 600 MHz): δ (ppm) 7.88 (d, J=2.6 Hz, 1H), 7.50-7.52 (m, 1H), 7.38 (s, 1H), 3.89 (d, J=7.4 Hz, 2H), 3.77 (br dd, J=11.2, 4.4 Hz, 2H), 3.50 (s, 3H), 3.15-3.23 (m, 2H), 2.01-2.09 (m, 3H), 1.84-1.96 (m, 1H), 1.27-1.34 (m, 2H), 1.11 (qd, J=12.2, 4.5 Hz, 2H). The filtrate from the above trituration (that furnished Batch 6) was concentrated in vacuo and triturated again with TBME. The solid was collected by filtration and dried in vacuo to furnish the title compound (Batch 8, 2.21 g) as an off-white solid. LCMS (System B): t_{RET}=0.74 min; MH⁺ 322, 324.

### Example 30a: Preparation of 5-(4-chloro-1-((tetrahydro-2H-pyran-4-yl)methyl)-1H-imidazol-2-yl)-1,3-dimethylpyridin-2(1H)-one hydrate

5-(4-chloro-1-((tetrahydro-2H-pyran-4-yl)methyl)-1H-imidazol-2-yl)-1,3-dimethylpyridin-2(1H)-one (for an example preparation, see Example 30) (9.2 g) was added to a 250 mL RB flask with water (100 mL) and stirred overnight at 30° C. The slurry was isolated by vacuum filtration on a Bichner funnel and the filtrate was recycled to wash the flask and product. The cake was air-dried overnight at ambient temperature and humidity to give the title compound as a white crystalline solid (9.1 g).

### Example 31: 5-(1-ethyl-1H-imidazol-5-yl)-1,3-dimethylpyridin-2(1H)-one

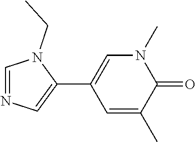 [SMILES: CCn1cncc1-c1cc(C)c(=O)n(C)c1]

A mixture of 1,3-dimethyl-5-(4,4,5,5-tetramethyl-1,3,2-dioxaborolan-2-yl)pyridin-2(1H)-one (commercially available from, for example, Milestone PharmaTech, 678 mg, 2.72 mmol), a 3:1 mixture of 4-bromo-1-ethyl-1H-imidazole and 5-bromo-1-ethyl-1H-imidazole (for an example preparation, see Intermediate 22, 476 mg, 2.72 mmol), potassium carbonate (1.88 g, 13.6 mmol) and bis(triphenylphosphine)palladium(II) chloride (191 mg, 0.272 mmol) in 1,2-dimethoxyethane (8 mL) and water (2 mL) was heated at 80° C. in a microwave for 2 h. The cooled reaction mixture was diluted with ethyl acetate (20 mL) filtered through Celite®. The filtrate was dried over sodium sulphate and evaporated. The residue was chromatographed [0-20% ethanol/ethyl acetate] to give the crude product which was repurified by High pH MDAP (Method B) to give the title compound as a colourless oil (12 mg). LCMS (System B): t_{RET}=0.55 min; MH⁺ 218.

### Example 32: rac-1-(4-chloro-1-((tetrahydro-2H-pyran-3-yl)methyl)-1H-imidazol-2-yl)-3,5-dimethylpyridin-4(1H)-one

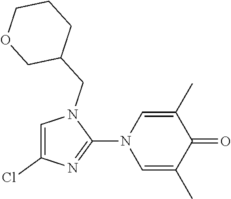 [SMILES: Cc1cn(-c2nc(Cl)cn2CC2CCCOC2)cc(C)c1=O]

Rac-2-bromo-4-chloro-1-((tetrahydro-2H-pyran-3-yl)methyl)-1H-imidazole (for an example preparation, see Intermediate 19, 100 mg, 0.358 mmol), 3,5-dimethylpyridin-4(1H)-one (132 mg, 1.073 mmol), copper(I) iodide (6.8 mg, 0.036 mmol) and potassium carbonate (99 mg, 0.715 mmol) were combined in DMSO (2 mL). The reaction mixture was purged with nitrogen and heated to 110° C. for 17 h. Further copper (I) iodide (6.8 mg, 0.036 mmol) was added to the reaction mixture and heating continued for a further 24 h. The reaction mixture was cooled and filtered through Celite®, washing with EtOAc (10 mL). The filtrate was washed with water (10 mL) and the aqueous re-extracted with EtOAc (2×10 mL). The combined organics were passed through a hydrophobic frit and the resulting filtrate concentrated in vacuo. The residue was redissolved in 1:1 solution of MeOH:DMSO and purified by MDAP (Method B) to afford the title compound as a white solid (22 mg). LCMS (System B): t_{RET}=0.76 min; MH⁺ 322, 324.

### Examples 33 and 34: methyl 2-(1,5-dimethyl-6-oxo-1,6-dihydropyridin-3-yl)-1-((tetrahydro-2H-pyran-4-yl)methyl)-1H-imidazole-4-carboxylate (Example 33) and methyl 2-(1,5-dimethyl-6-oxo-1,6-dihydropyridin-3-yl)-1-((tetrahydro-2H-pyran-4-yl)methyl)-1H-imidazole-5-carboxylate (Example 34)

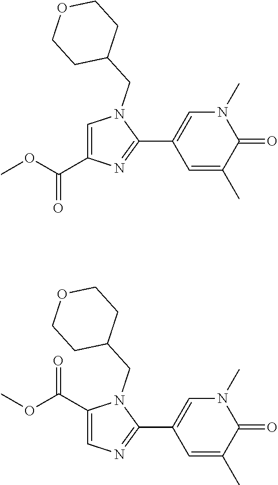 [SMILES: COC(=O)c1cn(CC2CCOCC2)c(-c2cc(C)c(=O)n(C)c2)n1 ; COC(=O)c1cnc(-c2cc(C)c(=O)n(C)c2)n1CC1CCOCC1]

Methyl 2-(1,5-dimethyl-6-oxo-1,6-dihydropyridin-3-yl)-1H-imidazole-4-carboxylate (for an example preparation, see Intermediate 23, 250 mg, 1.011 mmol) was suspended in DMF (10 mL) under nitrogen. Potassium carbonate (279 mg, 2.022 mmol), then 4-(bromomethyl)tetrahydro-2H-pyran (253 mg, 1.416 mmol) were added and the reaction heated to 100° C. over the weekend. The reaction was cooled and partitioned between EtOAc and water (20 mL each). The aqueous was re-extracted with EtOAc (20 mL) and the combined organics were dried with Na₂SO₄, filtered through a hydrophobic frit and concentrated in vacuo to yield an orange oil. The crude product was applied to a 10 g SNAP cartridge in the minimum of DCM and eluted with 20-100% (3:1 EtOAc:EtOH). The appropriate fractions were concentrated in vacuo to give crude product which was purified by MDAP (Method A) to give the title compounds as clear oils (24 mg, Example 33, and 2 mg, Example 34). LCMS (System B): t_{RET}=0.65 min; MH⁺ 346 (Example 33); LCMS (System B): t_{RET}=0.74 min; MH⁺ 346 (Example 34).

### Example 35: 2-(1,5-dimethyl-6-oxo-1,6-dihydropyridin-3-yl)-1-((tetrahydro-2H-pyran-4-yl)methyl)-1H-imidazole-4-carboxylic acid

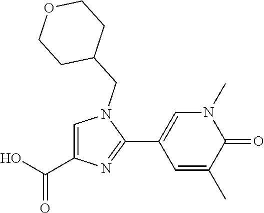 [SMILES: Cc1cc(-c2nc(C(=O)O)cn2CC2CCOCC2)cn(C)c1=O]

Methyl 2-(1,5-dimethyl-6-oxo-1,6-dihydropyridin-3-yl)-1-((tetrahydro-2H-pyran-4-yl)methyl)-1H-imidazole-4-carboxylate (for an example preparation, see Example 33, 50 mg, 0.145 mmol) was taken up in methanol (2 mL) and THF (2 mL). LiOH (0.724 mL, 0.724 mmol) was added and the reaction heated to 50° C. for 2 h. The reaction was cooled, acidified with 2N HCl and then concentrated in vacuo to give a yellow semi-solid. MDAP purification (Method A) gave the title compound as a cream solid (26 mg). LCMS (System A): t_{RET}=0.42 min; MH⁺ 332.

### Example 36: rac-5-(4-bromo-1-((tetrahydro-2H-pyran-3-yl)methyl)-1H-imidazol-2-yl)-1,3-dimethylpyridin-2(1H)-one

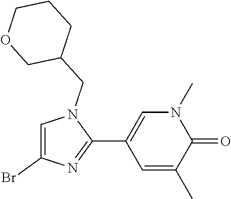 [SMILES: Cc1cc(-c2nc(Br)cn2CC2CCCOC2)cn(C)c1=O]

A mixture of rac-2,4-dibromo-1-((tetrahydro-2H-pyran-3-yl)methyl)-1H-imidazole (for an example preparation, see Intermediate 24, 60 mg, 0.185 mmol), 1,3-dimethyl-5-(4,4,5,5-tetramethyl-1,3,2-dioxaborolan-2-yl)pyridin-2(1H)-one (commercially available from, for example, Milestone PharmaTech, 60 mg, 0.241 mmol), tetrakis(triphenylphosphine)palladium(0) (1 mg, 0.926 μmol) and potassium carbonate (77 mg, 0.556 mmol) in 1,4-Dioxane (0.45 mL) and Water (0.15 mL) was heated in a microwave to 100° C. for 1 h. The reaction mixture was filtered through Celite® and washed with ethyl acetate (20 mL). The solvent was then evaporated in vacuo to afford an orange oil. The residue was redissolved in 1:1 solution of MeOH:DMSO and purified by MDAP (Method B) to afford the title compound as a white solid (27 mg). LCMS (System A): t_{RET}=0.74 min; MH⁺ 366, 368.

### Example 37: rac-1-(4-bromo-1-((tetrahydro-2H-pyran-3-yl)methyl)-1H-imidazol-2-yl)-3,5-dimethylpyridin-4(1H)-one

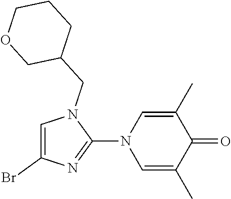 [SMILES: Cc1cn(-c2nc(Br)cn2CC2CCCOC2)cc(C)c1=O]

Rac-2,4-dibromo-1-((tetrahydro-2H-pyran-3-yl)methyl)-1H-imidazole (for an example preparation, see Intermediate 24, 55 mg, 0.170 mmol), 3,5-dimethylpyridin-4(1H)-one (63 mg, 0.509 mmol), copper(I) iodide (3. mg, 0.017 mmol) and potassium carbonate (47 mg, 0.339 mmol) were combined in DMSO (1.5 mL). The reaction mixture was purged with nitrogen and heated to 110° C. The reaction mixture was cooled and filtered through Celite® washing with EtOAc (10 mL). The filtrate was washed with water (10 mL) and the aqueous re-extracted with EtOAc (2×10 mL). The combined organics were passed through a hydrophobic frit and the resulting filtrate concentration in vacuo. The residue was redissolved in 1:1 solution of MeOH:DMSO and purified by MDAP (Method B) to afford the title compound as a white solid (16 mg). LCMS (System B): t_{RET}=0.78 min; MH⁺ 366, 368.

### Example 38: 1-(4-bromo-1-((tetrahydro-2H-pyran-4-yl)methyl)-1H-imidazol-2-yl)-3,5-dimethylpyridin-4(1H)-one

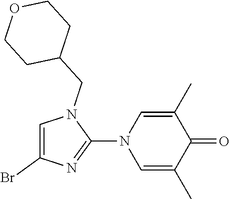 [SMILES: Cc1cn(-c2nc(Br)cn2CC2CCOCC2)cc(C)c1=O]

A 3:1 mixture of 2,4-dibromo-1-((tetrahydro-2H-pyran-4-yl)methyl)-1H-imidazole and 2,5-dibromo-1-((tetrahydro-2H-pyran-4-yl)methyl)-1H-imidazole (for an example preparation, see Intermediate 25, 60 mg, 0.185 mmol), 3,5-dimethylpyridin-4(1H)-one (68.4 mg, 0.556 mmol), copper(I) iodide (3.5 mg, 0.019 mmol) and potassium carbonate (51 mg, 0.370 mmol) were combined in DMSO (1.5 mL). The reaction mixture was purged with nitrogen and then heated to 110° C. for 19 h. Further copper (I) iodide (3.5 mg, 0.019 mmol) and potassium carbonate (51 mg, 0.370 mmol) were added and the reaction mixture stirred at 110° C. for a further 23 h. Additional copper(I) iodide (3.5 mg, 0.019 mmol) and potassium carbonate (51 mg, 0.370 mmol) were added and the reaction mixture stirred at 110° C. for a further 3 h. The reaction mixture was cooled and filtered through Celite®, washing with EtOAc (10 mL) and the resulting filtrate concentrated in vacuo to afford 28 mg of an orange oil. The residue was redissolved in 1:1 solution of MeOH:DMSO and purified by MDAP (Method B) to afford the title compound as a colourless oil (2 mg). LCMS (System B): t_{RET}=0.75 min; MH⁺ 366, 368.

### Example 39: 5-(4-bromo-1-((tetrahydro-2H-pyran-4-yl)methyl)-1H-imidazol-2-yl)-1,3-dimethylpyridin-2(1H)-one

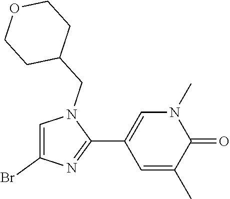 [SMILES: Cc1cc(-c2nc(Br)cn2CC2CCOCC2)cn(C)c1=O]

A 3:1 mixture of 2,4-dibromo-1-((tetrahydro-2H-pyran-4-yl)methyl)-1H-imidazole and 2,5-dibromo-1-((tetrahydro-2H-pyran-4-yl)methyl)-1H-imidazole (for an example preparation, see Intermediate 25, 50 mg, 0.154 mmol), 1,3-dimethyl-5-(4,4,5,5-tetramethyl-1,3,2-dioxaborolan-2-yl)pyridin-2(1H)-one (commercially available from, for example, Milestone PharmaTech, 50 mg, 0.201 mmol), tetrakis(triphenylphosphine)palladium(0) (0.9 mg, 0.772 μmol) and potassium carbonate (64 mg, 0.463 mmol) in 1,4-dioxane (0.39 mL) and water (0.13 mL) was heated in a microwave to 100° C. for 1 h. Additional potassium carbonate (64 mg, 0.463 mmol) and tetrakis(triphenylphosphine)palladium(0) (0.9 mg, 0.772 μmol) were added and the reaction mixture was heated to 100° C. in a microwave for a further 1 h. The reaction mixture was diluted with water (10 mL) and, extracted with EtOAc (3×10 ml). The organic layer was washed with brine solution (10 ml), then passed through a hydrophobic frit and concentrated in vacuo to afford a colourless oil. The resulting residue was dissolved in 3 mL DCM and was purified using a 12 g normal phase silica column, eluting with EtOAc (+1% NEt₃) to 25% ethanol to afford a colourless oil. The residue was further purified by preparative HPLC (XBridge Shield RP18 150×30 mm, 5 μm, rt, 0-99% MeOH/0.1% formic acid in water gradient over 41 min, 40 mL/min flow rate, UV detection summed signal from wavelength 210-350 nm) to give the title compound as a colourless oil (7 mg). LCMS (System B): t_{RET}=0.76 min; MH⁺ 366, 368.

### Example 40: rac-5-(1-((1-acetylpiperidin-3-yl)methyl)-4-bromo-1H-imidazol-2-yl)-1,3-dimethylpyridin-2(1H)-one

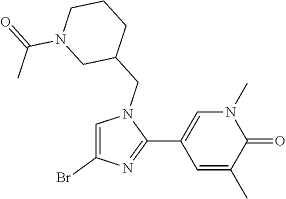 [SMILES: CC(=O)N1CCCC(Cn2cc(Br)nc2-c2cc(C)c(=O)n(C)c2)C1]

A mixture of rac-1-(3-((2,4-dibromo-1H-imidazol-1-yl)methyl)piperidin-1-yl)ethan-1-one (for an example preparation, see Intermediate 26, 64 mg, 0.175 mmol), 1,3-dimethyl-5-(4,4,5,5-tetramethyl-1,3,2-dioxaborolan-2-yl)pyridin-2(1H)-one (commercially available from, for example, Milestone PharmaTech, 44 mg, 0.175 mmol), tetrakis(triphenylphosphine)palladium(0) (1 mg, 0.877 μmol) and potassium carbonate (73 mg, 0.526 mmol) in 1,4-dioxane (0.450 mL) and water (0.15 mL) was heated in a microwave to 100° C. for 1 h. The reaction mixture was filtered through Celite® and washed with ethyl acetate (20 mL). The solvent was then evaporated in vacuo to afford an orange oil. The residue was redissolved in 1:1 solution of MeOH:DMSO and purified by MDAP (Method B) to afford crude product as a white solid. The residue was redissolved in 1:1 solution of MeOH:DMSO and repurified by MDAP (Method B) to afford the title compound as a colourless oil (15 mg). LCMS (System B): t_{RET}=0.72 min; MH⁺ 407, 409.

### Example 41: 1-(4-chloro-1-(1,3-dimethoxypropan-2-yl)-1H-imidazol-2-yl)-3,5-dimethylpyridin-4(1H)-one

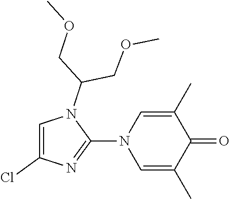 [SMILES: COCC(COC)n1cc(Cl)nc1-n1cc(C)c(=O)c(C)c1]

3,5-Dimethylpyridin-4(1H)-one (70.4 mg, 0.571 mmol), copper(I) iodide (3.6 mg, 0.019 mmol), potassium carbonate (52.6 mg, 0.381 mmol) and 2-bromo-4-chloro-1-(1,3-dimethoxypropan-2-yl)-1H-imidazole (for an example preparation, see Intermediate 13, 54 mg, 0.190 mmol) were combined with dry DMSO (0.5 mL). The reaction mixture was heated at 110° C. for 65 h. The reaction mixture was cooled and filtered through Celite® and washed with EtOAc (˜15 mL). The filtrate was washed with water (15 mL) and the aqueous layer extracted with EtOAc (3×10 mL). The combined organic layers were passed through a hydrophobic frit and the filtrate concentrated in vacuo to give the crude product. The crude product was dissolved in a 1:1 mixture of MeOH:DMSO and purified by MDAP (Method B) to afford the title compound as an off-white solid (11 mg). LCMS (System B): t_{RET}=0.84 min; MH⁺ 326, 328.

### Example 42: 1-(4-chloro-1-((tetrahydro-2H-pyran-4-yl)methyl)-1H-imidazol-2-yl)-3,5-dimethylpyridin-4(1H)-one

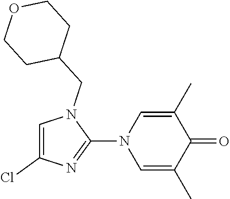 [SMILES: Cc1cn(-c2nc(Cl)cn2CC2CCOCC2)cc(C)c1=O]

3,5-Dimethylpyridin-4(1H)-one (132 mg, 1.073 mmol), copper(I) iodide (12 mg, 0.063 mmol), potassium carbonate (99 mg, 0.715 mmol) and 2-bromo-4-chloro-1-((tetrahydro-2H-pyran-4-yl)methyl)-1H-imidazole (for an example preparation, see Intermediate 21, 100 mg, 0.358 mmol) were combined with dry DMSO (1 mL). The reaction mixture was heated at 110° C., with stirring for 100 h. Copper iodide (12 mg, 0.063 mmol) was added to the reaction, which was left stirring for a further 24 h. The reaction mixture was cooled to room temperature and filtered through Celite®, washing with EtOAc (˜30 mL). The filtrate was washed with water (15 mL) and the aqueous layer extracted with EtOAc (3×15 mL). The combined organic layers were passed through a hydrophobic frit and the filtrate concentrated in vacuo to give a yellow oil. The crude product was dissolved in a 1:1 mixture of MeOH:DMSO (0.8 mL) and purified by MDAP (Method B) to afford the title compound as a white solid (14.5 mg). LCMS (System B): t_{RET}=0.73 min; MH⁺ 322, 324.

### Biological Data

### Time Resolved Fluorescence Resonance Energy Transfer (TR-FRET) Assay

Bromodomain binding was assessed utilising a time resolved fluorescent resonance energy transfer (TR-FRET) competition assay. To enable this approach a known, high affinity, pan-BET interacting small molecule was labeled with Alexa Fluor® 647, which is a far-red-fluorescent dye (Reference Compound X). Reference Compound X acts as a reporter of bromodomain binding and is the acceptor fluorophore component of the TR-FRET pair. Europium chelate, conjugated to an anti-6*His antibody, was utilised as the donor fluorophore in the TR-FRET pair (PerkinElmer AD0111). The anti-6*His antibody binds selectively to a six Histidine purification epitope added to the amino-terminus of each of the BET tandem bromodomain containing protein constructs used in this study. A TR-FRET signal is generated when the donor and acceptor fluorophores are in close proximity, between 20-80 Å, which is enabled in this assay by binding of Reference Compound X to the bromodomain containing protein.

### Reference Compound X: 4-((Z)-3-(6-((5-(2-((4S)-6-(4-chlorophenyl)-8-methoxy-1-methyl-4H-benzo[f][1,2,4]triazolo[4,3-a][1,4]diazepin-4-yl)acetamido)pentyl)amino)-6-oxohexyl)-2-((2E,4E)-5-(3,3-dimethyl-5-sulfo-1-(4-sulfobutyl)-3H-indol-1-ium-2-yl)penta-2,4-dien-1-ylidene)-3-methyl-5-sulfoindolin-1-yl)butane-1-sulphonate)

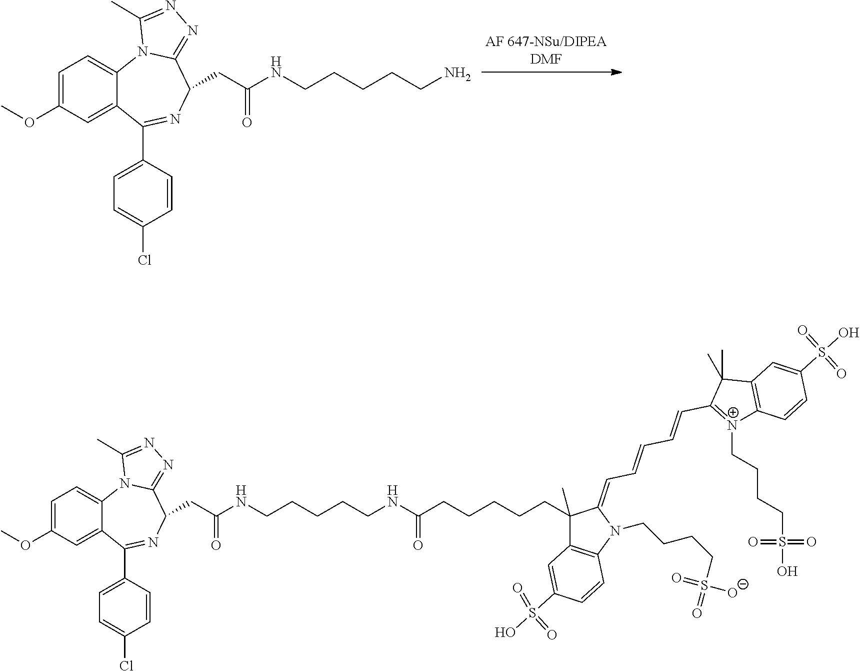 [SMILES: COc1ccc2c(c1)C(c1ccc(Cl)cc1)=N[C@@H](CC(=O)NCCCCCN)c1nnc(C)n1-2 ; COc1ccc2c(c1)C(c1ccc(Cl)cc1)=N[C@@H](CC(=O)NCCCCCNC(=O)CCCCCC1(C)/C(=C/C=C/C=C/C3=[N+](CCCCS(=O)(=O)O)c4ccc(S(=O)(=O)O)cc4C3(C)C)N(CCCCS(=O)(=O)[O-])c3ccc(S(=O)(=O)O)cc31)c1nnc(C)n1-2]

To a solution of N-(5-aminopentyl)-2-((4S)-6-(4-chlorophenyl)-8-methoxy-1-methyl-4H-benzo[f][1,2,4]triazolo[4,3-a][1,4]diazepin-4-yl)acetamide (for a preparation see Reference Compound J, WO2011/054848A1, 1.7 mg, 3.53 μmol) in DMF (40 μL) was added a solution of AlexaFluor647-ONSu (2.16 mg, 1.966 μmol) also in DMF (100 μL). The mixture was basified with DIPEA (1 μl, 5.73 μmol) and agitated overnight on a vortex mixer. The reaction mixture was evaporated to dryness. The solid was dissolved in MeCN/water/AcOH (5/4/1, <1 mL) filtered and was applied to a Phenomenex Jupiter C18 preparative column and eluted with the following gradient (A=0.1% trifluoroacetic acid in water, B=0.1% TFA/90% MeCN/10% water): Flow rate=10 mL/min., AU=20/10 (214 nm): 5-35%, t=0 min: B=5%; t=10 min: B=5%; t=100 min: B=35%; t=115 min: B=100% (Sep. grad: 0.33%/min)

The major component was eluted over the range 26-28% B but appeared to be composed of two peaks. The middle fraction (F1.26) which should contain “both” components was analysed by analytical HPLC (Spherisorb ODS2, 1 to 35% over 60 min): single component eluting at 28% B. Fractions F1.25/26&27 were combined and evaporated to dryness. Transferred with DMF, evaporated to dryness, triturated with dry ether and the blue solid dried overnight at <0.2 mbar: 1.54 mg. Analytical HPLC (Sphersisorb ODS2, 1 to 35% B over 60 min): MSM10520-1: [M+H]⁺ (obs): 661.8/—corresponding with M-29. This equates to [(M+2H)/2]⁺ for a calculated mass of 1320.984 which is M-29. This is a standard occurrence with the Alexa Fluor 647 dye and represents a theoretical loss of two methylene groups under the conditions of the mass spectrometer.

Assay Principle:

In order to generate a TR-FRET signal, donor fluorophore is excited by a laser at A337 nm, which subsequently leads to emission at A618 nm. If the acceptor fluorophore is in close proximity then energy transfer can occur, which leads to emission of Alexa Fluor® 647 at A665 nm. In the presence of competitor compound, Reference Compound X can be displaced from binding to the bromodomain. If displacement occurs, the acceptor fluorophore is no longer in proximity to the donor fluorophore, which prevents fluorescent energy transfer and, subsequently, a loss of Alexa Fluor® 647 emission at A665 nm.

The competition of the compounds of formula (I) with Reference Compound X for binding to the BET family (BRD2, BRD3, BRD4 and BRDT) was assessed using protein truncates spanning both bromodomain 1 (BD1) and bromodomain 2 (BD2). In order to monitor differential binding to either BD1 or BD2, single residue mutations of key tyrosines to alanine were made in the acetyl lysine binding pockets. To validate this approach, a double residue mutant tandem domain protein was produced for each of the BET family members. Utilising a Fluorescence Polarisation approach, binding affinities for each of the single and double mutants for Reference Compound X were determined. The affinities of the double mutant tandem proteins for Reference Compound X were greatly reduced in comparison to the non mutated, wild type tandem BET proteins (>1000 fold reduction in Kd). The affinities of the single mutated bromodomain tandem proteins for Reference Compound X were equi-potent with the corresponding non-mutated BET protein. These data demonstrated that single mutations of Tyrosine to Alanine reduce the Kd of the interaction between the mutated bromodomain and Reference Compound X by >1000 fold. In the TR-FRET competition assay, Reference Compound X is used at a concentration that is equivalent to the Kd for the non-mutated bromodomain, which ensures that no binding at the mutated bromodomain is detected.

Protein Production:

Recombinant Human Bromodomains [(BRD2 (1-473) (Y113A) and (Y386A), BRD3 (1-435) (Y73A) and (Y348A) BRD4 (1-477) (Y97A) and (Y390A) and BRDT (1-397) (Y66A) and (Y309A)] were expressed in E. coli cells (in pET15b vector for BRD2/3/4 and in pET28a vector for BRDT) with a 6-His tag at the N-terminal. The His-tagged Bromodomain pellet was resuspended in 50 mM HEPES (pH7.5), 300 mM NaCl, 10 mM imidazole & 1 μL/mL protease inhibitor cocktail and extracted from the E. coli cells using sonication and purified using a nickel sepharose high performance column, the proteins were washed and then eluted with a linear gradient of 0-500 mM imidazole with buffer 50 mM HEPES (pH7.5), 150 mM NaCl, 500 mM imidazole, over 20 column volumes. Final purification was completed by Superdex 200 prep grade size exclusion column. Purified protein was stored at −80° C. in 20 mM HEPES pH 7.5 and 100 mM NaCl. Protein identity was confirmed by peptide mass fingerprinting and predicted molecular weight confirmed by mass spectrometry.

Protocol for Bromodomain BRD2, 3, 4 and T, BD1+BD2 Mutant TR-FRET Competition Assays:

All assay components were dissolved in an assay buffer composing of 50 mM HEPES pH7.4, 50 mM NaCl, 5% Glycerol, 1 mM DTT and 1 mM CHAPS. Reference Compound X was diluted, in assay buffer containing 20 nM single mutant, tandem bromodomain containing protein, to a concentration equivalent to 2*Kd for this bromodomain. The solution containing bromodomain and Reference Compound X was added to dose response dilutions of test compound or DMSO vehicle (a maximum of 0.5% DMSO is used in this assay) in Greiner 384 well black low volume microtitre plates and subsequently incubated for 30 minutes at RT. An equal volume of 3 nM of anti-6*His Europium chelate was added to all wells, followed by a further 30 minute incubation at room temperature. TR-FRET was detected using a Perkin Elmer Multimode plate reader, by exciting the donor fluorophore at A337 nm and subsequently, after a delay of 50 μsecs, measuring emission of the donor and acceptor fluorophores at A615 nm and A665 nm, respectively. In order to control these assays, 16 wells each of uninhibited (DMSO vehicle) and inhibited (10*IC₅₀ concentrations of Example 11 of WO 2011/054846A1) reactions were included on every microtitre plate.

A four parameter curve fit of the following form was then applied:

y=a+((b−a)/(1+(10̂x/10̂c)̂d)

Where ‘a’ is the minimum, ‘b’ is the Hill slope, ‘c’ is the pIC₅₀ and ‘d’ is the maximum.

Results: All the Examples were tested in the above BRD4 assay and were found to have a mean pIC₅₀ in the range of 5.2 to 7.8 in the BRD4 BD1 assay and a mean pIC₅₀ in the range of 4.4 to 6.3 in the BRD4 BD2 assay. Example 30 was found to have a mean pIC₅₀ of 7.1 (n=22) in the BRD4 BD1 assay and a mean pIC₅₀ of 5.9 (n=16) in the BRD4 BD2 assay. Example 22 was found to have a mean pIC₅₀ of 7.3 in the BRD4 BD1 assay and a mean pIC₅₀ of 5.7 in the BRD4 BD2 assay.

Examples 1, 3, 5, 6, 7, 25, 29 and 30 were tested in the BRD2 and BRDT assays and were found to have a mean pIC₅₀ in the range of 5.1 to 7.9 in the BRD2 BD1 assay, a mean pIC₅₀ in the range of 4.3 to 6.0 in the BRD2 BD2 assay, a mean pIC₅₀ in the range of 4.9 to 7.4 in the BRDT BD1 assay, and a mean pIC₅₀ in the range of 4.6 to 5.7 in the BRDT BD2 assay. Example 1 had a mean pIC₅₀ of <4.3 in the BRDT BD2 assay. Examples 25, 29 and 30 were tested in the BRD3 assay and were found to have a mean pIC₅₀ in the range of 7.1 to 7.7 in the BRD3 BD1 assay, a mean pIC₅₀ in the range of 6.0 to 6.6 in the BRD3 BD2 assay.

Measurement of LPS Induced MCP-1 Production from Human Whole Blood

Activation of monocytic cells by agonists of toll-like receptors such as bacterial lipopolysaccharide (LPS) results in production of key inflammatory mediators including MCP-1. Such pathways are widely considered to be central to the pathophysiology of a range of auto-immune and inflammatory disorders. Blood is collected in a tube containing Sodium heparin (Leo Pharmaceuticals) (10 units of heparin/mL of blood). 96-well compound plates containing 1 μL test sample in 100% DMSO were prepared (two replicates on account of donor variability). 130 μL of whole blood was dispensed into each well of the 96-well compound plates and incubated for 30 min at 37° C., 5% CO₂. 10 μL of lipopolysaccharide (from Salmonella typhosa; L6386) made up in PBS (200 ng/mL final assay concentration) was added to each well of the compound plates. The plates were then placed in the humidified primary cell incubator for 18-24 hours at 37° C., 5% CO₂. 140 μL of PBS was added to all wells of the compound plates containing blood. The plates were then sealed and centrifuged for 10 mins at 2500 rpm. 25 μL of cell supernatant was placed in a 96-well MSD plate pre-coated with human MCP-1 capture antibody. The plates were sealed and placed on a shaker at 600 rpm for 1 hour (r.t). 25 μL of Anti-human MCP-1 antibody labelled with MSD SULFO-TAG™ reagent is added to each well of the MSD plate (stock 50× was diluted 1:50 with Diluent 100, final assay concentration is 1 μg/mL). The plates were then re-sealed and shaken for another hour before washing with PBS. 150 μL of 2×MSD Read Buffer T (stock 4×MSD Read Buffer T was diluted 50:50 with de-ionised water) was then added to each well and the plates read on the MSD Sector Imager 6000. Concentration response curves for each compound were generated from the data and an pIC₅₀ value was calculated. Results: All the Examples (with the exception of Examples 2 to 4, 31, 33, 34, 38 to 40) were tested in the above assay and were found to have a mean pIC₅₀ in the range of 4.7 to 7.5. Example 35 had a mean pIC₅₀ of <4.7. Example 30 had a mean pIC₅₀ of 6.9. Example 22 had a mean pIC₅₀ of 7.0. These data demonstrate that bromodomain inhibitors tested in the above whole blood assay inhibited the production of the key inflammatory mediator MCP-1.

### Trinitrophenol-Keyhole Limpet Hemocyanin (TNP-KLH) Induced Immunoglobulin-1 (IgG1) Production Mouse Assay

The T cell dependent mouse immunisation model is a mechanistic in vivo model representing immune activation to a T cell dependent antigen keyhole limpet haemocyanin 2, 4, 6 nitrophenol (KLH-TNP). Administration of KLH-TNP provokes an antibody response which involves fundamental immune cell interactions between T and B cells and dendritic cells. Example 30 was assayed for its ability to inhibit trinitrophenol-keyhole limpet hemocyanin (TNP-KLH) induced Immunoglobulin-1 (IgG1) production in mice. Male CD1 mice (Charles River Laboratories), were allocated into 4 groups (n=7 per group) and assigned a specific dosing regime. The treatments were either a single oral administration of 1% (w/v) methylcellulose (aq 400), or compound at 1.5, 5 or 15 mg/kg twice daily (BID) at 0 and 4 h, over a 14 day dosing period. On day 1 of the study, each mouse received a single bolus intraperitoneal (ip) administration of TNP-KLH (100 ug/kg) 1 hour after oral administration of compound. Serial blood samples were collected via the tail veil at 1, 4 and 8 hour on day 1, and at 1 h on days 7 and 11 post initial daily oral administration or via cardiac puncture (terminal sample) on day 14. The serum harvested from the blood samples on days 7, 11 and 14 was frozen at −80° C. No adverse side effects were observed in any of the treatment groups, throughout the in life phase. On the day of analysis, the serum was thawed to room temperature and levels of IgG1 were measured using a TNP ELISA (developed in-house) and read on a SpectraMax 190 spectrophotometer (Molecular Devices, CA). The mean IgG1 values were generated and the mean percent IgG1 reduction on day 14 following treatment with compound was calculated compared to the corresponding vehicle treated group. Levels of significance were calculated by analysis of variance (ANOVA) followed by Dunnett's multiple comparison t-test using Graphpad Prism version 5.04 (Graphpad Software, San Diego, Calif.). Statistical differences were determined as ***P<0.01. Results are shown in Table 1.

The anti-inflammatory activity demonstrated in this model is considered representative of a key mechanism in vivo, supporting progression for the treatment of autoimmune and inflammatory conditions.

TABLE 1
Efficacy of Example 30 in the TNP-KLH-induced IgG1 production
mouse assay

| | Dose Group | | | |
|---|---|---|---|---|
| | | Example 30 | Example 30 | |
| | | 1.5 mg/kg, | 5.0 mg/kg, | Example 30 |
| Parameter | Vehicle | BID | BID | 15 mg/kg, BID |
| Day 14 IgG1 | 5.86 ± 3.44 | 5.01 ± 3.30 | 0.85 ± 1.18 | 0.03 ± 0.03 |
| (μg/mL) | | | | |
| % reduction | — | 0 | 86*** | 99*** |
| in IgG1 | | | | |
| from vehicle | | | | |

1. A compound of formula (I), or a salt thereof: 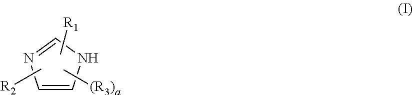
wherein
R₁ represents 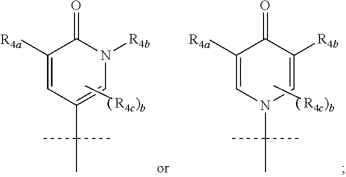
R₂ is hydrogen, C₁₋₆alkyl, C₁₋₆alkoxy, C₃₋₇cycloalkyl, heterocycloalkyl or —CHR₅(CH₂)_{c}R₆;
each R₃ is independently selected from the group consisting of halogen, —CN, C₁₋₃alkyl, C₁₋₃alkoxy, —NO₂, —CONR₇R, —NR₇COR₈, —OCOR₈, —CO₂R, —SO₂NR₇R, —NR₇SO₂R, —SO₂R₈, —R₈, —NR₇R₈, and —OR₈, with the proviso that when a is 2, one R₃ is selected from the group consisting of halogen, —CN, C₁₋₃alkyl and C₁₋₃alkoxy;
R₄ₐ is hydrogen, C₁₋₃alkyl, C₁₋₃alkoxy, halogen, —CN, —OH, —NR₉R₁₀;
R_{4b} is hydrogen or C₁₋₃alkyl;
each R_{4c} is independently selected from the group consisting of C₁₋₃alkyl, C₁₋₃alkoxy, halogen, —CN, —OH, and —NR₉R₁₀;
R₅ is hydrogen, C₁₋₃alkyl, or —(CH₂)_{d}OR₁₁;
R₆ is hydrogen, C₁₋₃alkyl, —(CH₂)_{d}OR₁₁, C₃₋₇cycloalkyl, or heterocycloalkyl, wherein the C₁₋₃alkyl, —(CH₂)_{d}OR₁₁, C₃₋₇cycloalkyl, or heterocycloalkyl group can be optionally substituted with one or two substituents independently selected from the group consisting of C₁₋₃alkyl, C₁₋₃alkoxy, halogen, —CH₂OH, —COOH, and —COCH₃;
R₇ is hydrogen or C₁₋₃alkyl and R₈ is —Y—Z, or when R₃ is —CONR₇R, R₇ and R₈ together with the nitrogen to which they are attached may form a heterocycloalkyl, wherein the heterocycloalkyl group can be optionally substituted with one or two groups independently selected from C₁₋₃alkyl, halogen, —NH₂, —CH₂NH₂, —CO₂H, —OH, —CN, and —CH₂OH;
Y is a bond or C₁₋₃alkylene, wherein the C₁₋₃alkylene group can be optionally substituted with one or two groups independently selected from C₁₋₃alkyl;
Z is hydrogen, C₁₋₃alkyl, C₃₋₇cycloalkyl, heterocycloalkyl, aryl, heteroaryl, —SO₂NR₁₂R₁₃, —NR₁₂SO₂R₁₃, —SO₂R₁₂, or —NR₁₂R₁₃, wherein the C₁₋₃alkyl, C₃₋₇cycloalkyl, heterocycloalkyl, aryl or heteroaryl group can be optionally substituted with one or two groups independently selected from C₁₋₃alkyl, C₁₋₃alkoxy, halogen, —NH₂, —CH₂NH₂, —CO₂H, —OH, —CN, and —CH₂OH;
R₉ is hydrogen or CH₃;
R₁₀ is hydrogen or C₁₋₃alkyl;
R₁₁ is hydrogen or C₁₋₃alkyl;
R₁₂ is hydrogen or C₁₋₃alkyl;
R₁₃ is hydrogen or C₁₋₃alkyl;
a represents 0, 1 or 2;
b represents 0, 1 or 2; and
each c and d independently represent 0 or 1.

2-3. (canceled)

4. The compound according to claim 1, comprising a compound of formula (Ia), or a salt thereof: 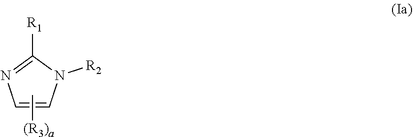
wherein R₁, R₂, R₃ and a are as defined in claim 1.

5. The compound or salt according to claim 1, wherein R₁ represents

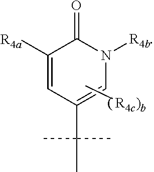

6-7. (canceled)

8. The compound or salt according to claim 1, wherein R₂ represents the group —CHR₅(CH₂)_{c}R₆.

9. (canceled)

10. The compound or salt according to claim 8, wherein R₅ is —(CH₂)_{d}OR₉.

11-14. (canceled)

15. The compound or salt according to claim 1, wherein R₂ is selected from the group consisting of: 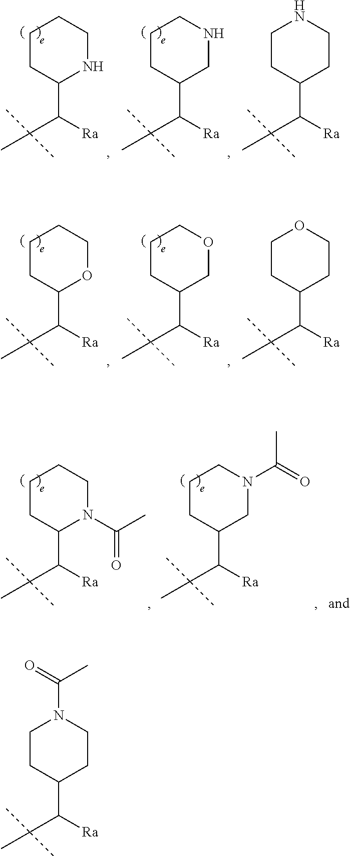
wherein Ra is hydrogen or C₁₋₃ alkyl; and e is 0 or 1.

16. The compound or salt according to claim 1, wherein R₂ is —CHR₅(CH₂)_{c}R₆, R₅ is —(CH₂)_{d}OR₉, b is 0 and R₆ is —(CH₂)_{d}OR₉.

17. (canceled)

18. The compound or salt according to claim 1, wherein R₄ₐ is CH₃ or —OCH₃.

19. (canceled)

20. The compound or salt according to claim 1, wherein R_{4b} is C₁₋₃alkyl.

21-23. (canceled)

24. The compound or salt according to claim 1, wherein a is 1 and R₃ is selected from the group consisting of halogen, —CN, C₁₋₃alkyl, and C₁₋₃alkoxy.

25-29. (canceled)

30. The compound according to claim 1, which is selected from the group consisting of:
5-(1H-imidazol-2-yl)-1,3-dimethylpyridin-2(1H)-one;
5-(4-bromo-1-ethyl-1H-imidazol-2-yl)-1,3-dimethylpyridin-2(1H)-one;
5-(1-(cyclopropylmethyl)-1H-imidazol-2-yl)-1,3-dimethylpyridin-2(1H)-one;
5-(4-bromo-1-(cyclopropylmethyl)-1H-imidazol-2-yl)-1,3-dimethylpyridin-2(1H)-one;
5-(1-isobutyl-1H-imidazol-2-yl)-1,3-dimethylpyridin-2(1H)-one;
1,3-dimethyl-5-(1-((tetrahydro-2H-pyran-4-yl)methyl)-1H-imidazol-2-yl)pyridin-2(1H)-one;
1,3-dimethyl-5-(1-((tetrahydro-2H-pyran-2-yl)methyl)-1H-imidazol-2-yl)pyridin-2(1H)-one;
(R)-1,3-dimethyl-5-(1-((tetrahydro-2H-pyran-2-yl)methyl)-1H-imidazol-2-yl)pyridin-2(1H)-one;
(S)-1,3-dimethyl-5-(1-((tetrahydro-2H-pyran-2-yl)methyl)-1H-imidazol-2-yl)pyridin-2(1H)-one;
1,3-dimethyl-5-(1-(piperidin-4-ylmethyl)-1H-imidazol-2-yl)pyridin-2(1H)-one;
1,3-dimethyl-5-(1-((tetrahydrofuran-2-yl)methyl)-1H-imidazol-2-yl)pyridin-2(1H)-one;
5-(1-(2-methoxyethyl)-1H-imidazol-2-yl)-1,3-dimethylpyridin-2(1H)-one;
5-(1-(1,3-dimethoxypropan-2-yl)-1H-imidazol-2-yl)-1,3-dimethylpyridin-2(1H)-one;
Methyl 2-(1,5-dimethyl-6-oxo-1,6-dihydropyridin-3-yl)-1H-imidazole-5-carboxylate;
5-(5-chloro-1H-imidazol-2-yl)-1,3-dimethylpyridin-2(1H)-one;
2-(1,5-dimethyl-6-oxo-1,6-dihydropyridin-3-yl)-1H-imidazole-5-carboxamide;
2-(1,5-dimethyl-6-oxo-1,6-dihydropyridin-3-yl)-1H-imidazole-4,5-dicarbonitrile;
5-(1-(1,3-dimethoxypropan-2-yl)-4,5-dimethyl-1H-imidazol-2-yl)-1,3-dimethylpyridin-2(1H)-one;
5-(4-(4-bromophenyl)-1-(1,3-dimethoxypropan-2-yl)-1H-imidazol-2-yl)-1,3-dimethylpyridin-2(1H)-one;
5-(4-chloro-1-((tetrahydro-2H-pyran-2-yl)methyl)-1H-imidazol-2-yl)-1,3-dimethylpyridin-2(1H)-one;
(R)-5-(4-chloro-1-((tetrahydro-2H-pyran-2-yl)methyl)-1H-imidazol-2-yl)-1,3-dimethylpyridin-2(1H)-one;
(S)-5-(4-chloro-1-((tetrahydro-2H-pyran-2-yl)methyl)-1H-imidazol-2-yl)-1,3-dimethylpyridin-2(1H)-one;
5-(5-chloro-1-((tetrahydro-2H-pyran-2-yl)methyl)-1H-imidazol-2-yl)-1,3-dimethylpyridin-2(1H)-one;
(R)-5-(5-chloro-1-((tetrahydro-2H-pyran-2-yl)methyl)-1H-imidazol-2-yl)-1,3-dimethylpyridin-2(1H)-one;
(S)-5-(5-chloro-1-((tetrahydro-2H-pyran-2-yl)methyl)-1H-imidazol-2-yl)-1,3-dimethylpyridin-2(1H)-one;
5-(5-chloro-1-((tetrahydro-2H-pyran-4-yl)methyl)-1H-imidazol-2-yl)-1,3-dimethylpyridin-2(1H)-one;
5-(5-chloro-1-((tetrahydro-2H-pyran-3-yl)methyl)-1H-imidazol-2-yl)-1,3-dimethylpyridin-2(1H)-one;
(R)-5-(5-chloro-1-((tetrahydro-2H-pyran-3-yl)methyl)-1H-imidazol-2-yl)-1,3-dimethylpyridin-2(1H)-one;
(S)-5-(5-chloro-1-((tetrahydro-2H-pyran-3-yl)methyl)-1H-imidazol-2-yl)-1,3-dimethylpyridin-2(1H)-one;
5-(4-chloro-1-(1,3-dimethoxypropan-2-yl)-1H-imidazol-2-yl)-1,3-dimethylpyridin-2(1H)-one;
5-(1-((1-acetylpiperidin-3-yl)methyl)-5-chloro-1H-imidazol-2-yl)-1,3-dimethylpyridin-2(1H)-one;
(R)-5-(1-((1-acetylpiperidin-3-yl)methyl)-5-chloro-1H-imidazol-2-yl)-1,3-dimethylpyridin-2(1H)-one;
(S)-5-(1-((1-acetylpiperidin-3-yl)methyl)-5-chloro-1H-imidazol-2-yl)-1,3-dimethylpyridin-2(1H)-one;
5-(1-((1-acetylpiperidin-3-yl)methyl)-4-chloro-1H-imidazol-2-yl)-1,3-dimethylpyridin-2(1H)-one;
(R)-5-(1-((1-acetylpiperidin-3-yl)methyl)-4-chloro-1H-imidazol-2-yl)-1,3-dimethylpyridin-2(1H)-one;
(S)-5-(1-((1-acetylpiperidin-3-yl)methyl)-4-chloro-1H-imidazol-2-yl)-1,3-dimethylpyridin-2(1H)-one;
5-(1-((1-acetylpiperidin-3-yl)methyl)-4-chloro-1H-imidazol-2-yl)-1,3-dimethylpyridin-2(1H)-one;
(R)-5-(1-((1-acetylpiperidin-3-yl)methyl)-4-chloro-1H-imidazol-2-yl)-1,3-dimethylpyridin-2(1H)-one
(S)-5-(1-((1-acetylpiperidin-3-yl)methyl)-4-chloro-1H-imidazol-2-yl)-1,3-dimethylpyridin-2(1H)-one
5-(4-chloro-1-((tetrahydro-2H-pyran-3-yl)methyl)-1H-imidazol-2-yl)-1,3-dimethylpyridin-2(1H)-one;
(R)-5-(4-chloro-1-((tetrahydro-2H-pyran-3-yl)methyl)-1H-imidazol-2-yl)-1,3-dimethylpyridin-2(1H)-one;
(S)-5-(4-chloro-1-((tetrahydro-2H-pyran-3-yl)methyl)-1H-imidazol-2-yl)-1,3-dimethylpyridin-2(1H)-one;
5-(4-chloro-1-((tetrahydro-2H-pyran-3-yl)methyl)-1H-imidazol-2-yl)-1,3-dimethylpyridin-2(1H)-one;
5-(4-chloro-1-((tetrahydro-2H-pyran-4-yl)methyl)-1H-imidazol-2-yl)-1,3-dimethylpyridin-2(1H)-one;
5-(1-ethyl-1H-imidazol-5-yl)-1,3-dimethylpyridin-2(1H)-one;
rac-1-(4-chloro-1-((tetrahydro-2H-pyran-3-yl)methyl)-1H-imidazol-2-yl)-3,5-dimethylpyridin-4(1H)-one;
methyl 2-(1,5-dimethyl-6-oxo-1,6-dihydropyridin-3-yl)-1-((tetrahydro-2H-pyran-4-yl)methyl)-1H-imidazole-4-carboxylate;
methyl 2-(1,5-dimethyl-6-oxo-1,6-dihydropyridin-3-yl)-1-((tetrahydro-2H-pyran-4-yl)methyl)-1H-imidazole-5-carboxylate;
2-(1,5-dimethyl-6-oxo-1,6-dihydropyridin-3-yl)-1-((tetrahydro-2H-pyran-4-yl)methyl)-1H-imidazole-4-carboxylic acid;
rac-5-(4-bromo-1-((tetrahydro-2H-pyran-3-yl)methyl)-1H-imidazol-2-yl)-1,3-dimethylpyridin-2(1H)-one;
rac-1-(4-bromo-1-((tetrahydro-2H-pyran-3-yl)methyl)-1H-imidazol-2-yl)-3,5-dimethylpyridin-4(1H)-one;
1-(4-bromo-1-((tetrahydro-2H-pyran-4-yl)methyl)-1H-imidazol-2-yl)-3,5-dimethylpyridin-4(1H)-one;
5-(4-bromo-1-((tetrahydro-2H-pyran-4-yl)methyl)-1H-imidazol-2-yl)-1,3-dimethylpyridin-2(1H)-one;
rac-5-(1-((1-acetylpiperidin-3-yl)methyl)-4-bromo-1H-imidazol-2-yl)-1,3-dimethylpyridin-2(1H)-one;
1-(4-chloro-1-(1,3-dimethoxypropan-2-yl)-1H-imidazol-2-yl)-3,5-dimethylpyridin-4(1H)-one; and
1-(4-chloro-1-((tetrahydro-2H-pyran-4-yl)methyl)-1H-imidazol-2-yl)-3,5-dimethylpyridin-4(1H)-one;
or a salt thereof.

31. The compound according to claim 1, which is 5-(4-chloro-1-((tetrahydro-2H-pyran-4-yl)methyl)-1H-imidazol-2-yl)-1,3-dimethylpyridin-2(1H)-one, of formula: 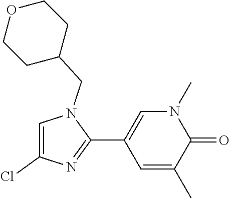 [SMILES: Cc1cc(-c2nc(Cl)cn2CC2CCOCC2)cn(C)c1=O]
or a salt thereof.

32. The compound according to claim 1, which is 5-(4-chloro-1-(1,3-dimethoxypropan-2-yl)-1H-imidazol-2-yl)-1,3-dimethylpyridin-2(1H)-one, of formula: 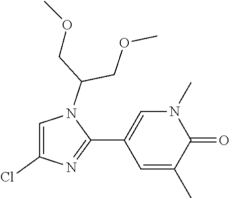 [SMILES: COCC(COC)n1cc(Cl)nc1-c1cc(C)c(=O)n(C)c1]
or a salt thereof.

33. The compound or salt according to claim 1, which is in the form of a pharmaceutically acceptable salt.

34. The compound according to claim 1, which is 5-(4-chloro-1-((tetrahydro-2H-pyran-4-yl)methyl)-1H-imidazol-2-yl)-1,3-dimethylpyridin-2(1H)-one monohydrate, of formula:

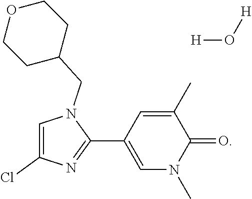

35. (canceled)

36. The compound according to claim 34 which is in crystalline form and which has one or more of the following:
a) an X-ray powder diffraction pattern (XRPD) substantially as shown in FIG. 1;
b) an X-ray powder diffraction pattern (XRPD) with specific peaks at 2θ values, +0.1° 2θ experimental error, of 10.0, 12.4, 13.1, 14.8, 15.8, 17.9, 19.6, 20.2, 21.2, 23.3, and 24.4 degrees;
c) a FT Raman spectrum substantially as shown in FIG. 2.

37. The compound according to claim 1, which is in the form of a free base.

38. A pharmaceutical composition comprising the compound or salt according to claim 1, and one or more pharmaceutically acceptable excipients.

39-41. (canceled)

42. A method of treatment of an autoimmune or inflammatory disease or cancer, which method comprises administering to a human subject in need thereof, a therapeutically effective amount of the compound or salt according to claim 1.

43. A method of treatment of rheumatoid arthritis, which method comprises administering to a human subject in need thereof, a therapeutically effective amount of the compound or salt according to claim 1.
